(19) United States
(12) Patent Application Publication
(10) Pub. No.: US 2026/0347475 A1
(43) Pub. Date: Nov. 5, 2026

(54) NOVEL PD1-TARGETED IL-2 IMMUNOCYTOKINE AND VITOKINE FUSIONS

(71) Applicant: CUGENE INC., Waltham, MA (US)

(72) Inventors: Yue-Sheng LI, Thousand Oaks, CA (US); Lingyun RUI, Weston, MA (US); Jing XU, Waltham, MA (US)

(21) Appl. No.: 19/110,022

(22) PCT Filed: Sep. 5, 2023
(86) PCT No.: PCT/US2023/031962

§ 371 Date: Mar. 7, 2025

Related U.S. Application Data
(60) Provisional application No. 63/404,608, filed on Sep. 8, 2022.

(51) Int. Cl. C07K 16/28; A61K 38/00; A61P 35/00; C07K 14/55

(52) U.S. Cl. CPC C07K 16/2818; A61P 35/00; C07K 14/55; A61K 38/00

(57) ABSTRACT

The present disclosure provides novel PD1 Ab-IL-2 immunocytokines and VitoKine compositions that aim to target a potency-attenuated or a bio-activable IL-2 directly to tumor-infiltrating lymphocytes to reduce systemic mechanism-based toxicities and lead to broader therapeutic utility for IL-2 for the treatment of cancer. For PD1 Ab-IL-2 VitoKine, IL-2 will remain inert until activated locally by proteases that are upregulated in diseased tissues, this will prevent over-activation of the pathway and reduce undesirable “on-target” “off tissue” toxicities, and significantly decrease the potential antigen or target sink, and thus, prolong the in vivo half-life and result in improved biodistribution, bioavailability and therapeutic efficacy. Additionally, PD1 antibodies capable of blocking PD1 and reversing T-cell anergy or exhaustion may further synergize with IL-2 anticancer immune response.

### RELATED APPLICATIONS

This application claims benefit of U.S. Provisional Application No. 63/404,608, filed on Sep. 8, 2022, incorporated in its entirety by reference herein.

### REFERENCE TO AN ELECTRIC SEQUENCE LISTING

The contents of the electronic sequence listing (SeqListing-CUGENE PD1 AB-IL-2.xml; Size: 216 Kilobytes; Production Date: Sep. 5, 2023) is herein incorporated by reference in its entirety.

### TECHNICAL FIELD

As cancer has been traditionally treated by chemotherapy, radiation, targeted therapies and surgery, a fifth pillar of cancer treatment, immunotherapy, has emerged over the recent years and revolutionized the war on cancer. The benchmark for the immunotherapy drugs has been established by the development of T cell checkpoint (CTLA-4 and PD1/PD-L1) inhibitors. It has been demonstrated that these therapies effectively expand and reactivate the pool of tumor-specific T cells leading to objective response rates of up to 50% in patients with certain cancers.

Interleukin 2 (IL-2) was the first growth factor described for T cells. The ability of IL-2 to expand lymphocyte populations in vivo and to increase the effector functions of these cells confers antitumor effects to IL-2 and led to the approval of high-dose recombinant IL-2 for certain metastatic cancers. While it demonstrated durable responses in approximately 10% of patients, IL-2 cancer immunotherapy is associated with multiple problems, including severe toxicity caused by the induction of vascular leak syndrome (VLS), tumor tolerance caused by the induction of activation-induced cell death (AICD), and immunosuppression caused by activation of Treg cells.

Several approaches have been taken to overcome the challenges inherent in IL-2 immunotherapy. One such approach to counter the systematic toxicity involves localizing cytokine activity to cancer cells and their surrounding tissues by tumor-targeted IL-2 immunocytokines, constructed by fusion of IL-2 to antibodies specific for tumor-associated antigens. However, this strategy lacks the ability to specifically target effector T cells within the tumor microenvironment (TME), which are pertinent to anticancer immunity. This gap in intratumoral T cell targeting may be filled by fusing IL-2 to an anti-programmed cell death protein 1 (PD1) antibody. PD1 (also known as CD279) is highly expressed on tumor-infiltrating lymphocytes (TILs), and PD1 antibody IL-2 immunocytokine enables IL-2 to be directly targeted to TILs. It displays elevated avidity toward intratumoral CD8+ T cells, rather than Treg cells or peripheral CD4+ and CD8+ T cells. This strategy thus further improves IL-2 anticancer immunity while reducing systemic toxicity.

In addition to targeting IL-2 directly to TILs to improve IL-2 anticancer immunity, PD1 antibodies capable of blocking PD1 and reversing T-cell anergy or exhaustion may synergize with IL-2, further boosting its anticancer immune response. Hence, it is desirable to construct a PD1 Ab-IL-2 immunocytokine with PD1 antibodies of superior target-binding and PD1 blocking capabilities. Among the various globally marketed PD1 blocking antibodies, which have fundamentally transformed the field of cancer immunotherapy, pembrolizumab (Keytruda®; Merck Sharp & Dohme Corp.) has received remarkable attention due to its high effectiveness and approvals for treating a wide variety of cancer types. While pembrolizumab exhibits superior target binding and blocking capabilities, it has several sequence liabilities, including a relatively low degree of humanness that could raise immunogenicity concerns, and high hydrophobicity that tends to increase its aggregation propensity. It is thus preferable to optimize pembrolizumab to mitigate its sequence liabilities while fully maintaining its biological activity. The resulting optimized sequence is expected to improve the developability of PD1 Ab-IL-2 fusion proteins.

Importantly, fusion of a PD1 Ab with a fully active IL-2 moiety may override the intended antibody-mediated targeting, localizing the fusion protein to IL-2 receptor-expressing cells in the peripheral instead of TILs in tumors. As such, to improve target specificity and selectivity, one approach is to prepare a fusion using an IL-2 moiety with attenuated IL-2Rβγ activity to establish a stoichiometric balance between the cytokine and antibody components. Additionally, decreasing the cytokine potency can potentially alleviate pathway over-activation as well as mitigate antigen sink and target-mediated deposition.

Another related but more sophisticated strategy to improve target specificity and selectivity is the application of the VitoKine platform disclosed by the current inventors in WO2019246392 and WO2021119516. In a VitoKine construct, the activity of the IL-2 moiety will remain inert or minimal until activated locally by proteases that are upregulated in or around tumors. By doing so, the binding of the IL-2 moiety to its receptors in the peripheral or on the cell-surface of non-diseased cells can be markedly limited. This can help prevent pathway over-activation and reduce undesirable “on-target” “off tissue” toxicity, and the improved safety profile of VitoKines may permit human dose levels within the effective range of a PD1 antibody. Additionally, the inertness of the IL-2 moiety prior to protease activation will significantly decrease the potential antigen or target sink, and thus, prolong the in vivo half-life and result in improved biodistribution and bioavailability at intended sites of therapy.

### DISCLOSURE OF THE INVENTION

In one aspect, the present invention provides a novel PD1-targeted bio-activable IL-2 immunocytokine (referred to herein as PD1 Ab-IL-2 VitoKine) that aims to target a bio-activable IL-2 directly to tumor-infiltrating lymphocytes. The activity of the IL-2 moiety will remain nearly inert or minimal until activated locally by proteases that are upregulated in tumors, which will limit binding of the IL-2 moiety to its receptors in the peripheral or on the cell-surface of non-diseased cells or normal tissues. This can help prevent pathway over-activation, reduce undesirable “on-target” “off tissue” toxicity, and minimize unwanted target sink.

In another aspect, the present invention provides novel PD1-targeted IL-2 immunocytokines that aim to target an activity-modulated IL-2 domain directly to tumor-infiltrating lymphocytes. The attenuated IL-2 activity is expected to facilitate establishing stoichiometric balance between the cytokine and antibody arms, help to alleviate pathway over-activation, and mitigate antigen sink and target-mediated deposition.

The strategy specifically targets effector T cells within the tumor microenvironment (TME) that are pertinent to anticancer immunity. By implementing this strategy, the ability of IL-2 to expand lymphocyte populations and augment their effector functions is synergized with the function of PD1 blocking antibody in reversing T-cell anergy or exhaustion. This approach, particularly when potency-attenuated or bio-activatable IL-2 is used, reduces systemic mechanism-based toxicities, leading to broader therapeutic utility of IL-2 for cancer treatment, and improves biodistribution and bioavailability at the intended sites of therapy.

In various embodiments, the PD1-targeted bio-activable IL-2 immunocytokine is referred to as PD1 Ab-IL-2 VitoKine herein. In various embodiments, the VitoKine platform disclosed in WO2019246392 and WO2021119516 by the current inventors is defined by the constructs as depicted in FIG. 1 and one of the proposed methods of activation as depicted in FIG. 2. In various embodiments, PD1 Ab-IL-2 VitoKine of the present invention is more specifically defined by the construct illustrated in FIG. 3A. Referring to FIG. 3A, the PD1 Ab-IL-2 VitoKine of the present invention comprises a PD1 blocking antibody, a monovalent IL-2 domain (the active moiety domain) with its N-terminus fused to the C-terminus of a heterodimeric Fc chain of the PD1 antibody via the L1 linker and its C-terminus fused to the N-terminus of IL-2Rα sushi domains (the concealing moiety domain) via the L2 linker.

In various embodiments, the variable domains of the PD1 blocking antibodies of the present invention were optimized from the variable domains of pembrolizumab by introducing germline sequence substitutions to the CDR residues, introducing germline sequence substitutions to the framework somatic mutations, and/or adopting the most prevalent and better behaving VH3 human germline family sequence as the acceptor framework. In various embodiments, the PD1 blocking antibodies have a high affinity for the human PD1 protein as set forth in SEQ ID NO: 1, function to inhibit PD1 with equal or comparable potency as pembrolizumab, exhibit higher sequence similarity scores to its closest human germline sequence than pembrolizumab, thereby indicating an improved degree of humanness, and are predicted to have lower hydrophobicity than pembrolizumab, which in turn reduce their propensity to aggregate.

In various embodiments, the PD1 blocking antibody comprises a light chain variable region with the sequence set forth in SEQ ID NO: 3, and a heavy chain variable region with the sequence set forth in SEQ ID NO: 7. In various embodiments, the PD1 blocking antibody comprises a light chain variable region with the sequence set forth in SEQ ID NO: 3, and a heavy chain variable region with the sequence set forth in SEQ ID NO: 9. In various embodiments, the PD1 blocking antibody comprises a light chain variable region with the sequence set forth in SEQ ID NO: 3, and a heavy chain variable region with the sequence set forth in SEQ ID NO: 11. In various embodiments, the PD1 blocking antibody comprises a light chain variable region with the sequence set forth in SEQ ID NO: 3, and a heavy chain variable region with the sequence set forth in SEQ ID NO: 13. In various embodiments, the PD1 blocking antibody comprises a light chain variable region with the sequence set forth in SEQ ID NO: 3, and a heavy chain variable region with the sequence set forth in SEQ ID NO: 18.

In various embodiments, the PD1 targeted-IL-2 immunocytokine is defined by the constructs as depicted in FIG. 3B. In various embodiments, the potency-modulated IL-2 of the PD1-targeted IL-2 immunocytokine is an IL-2 variant (or mutant) comprising a sequence derived from the sequence of the mature human IL-2 polypeptide (also referred to herein as huIL-12 or IL-2 wild type (w/t)) as set forth in SEQ ID NO: 116 comprising one or more amino acid substitution, deletion, or insertion. In various embodiments, the amino acid change is one or more amino acid substitutions at position 19, 65, 125 or 126 of SEQ ID NO: 116. In various embodiments, the amino acid change is the substitution of L to D or H or N or P or Q or R or S or Y at position 19, P to G or E or H or R or A or K or N or Q at position 65, C to I at position 125, Q to A or D or E or F or G or H or I or K or L or M or N or P or R or S or T or V or W or Y at position 126, of the mature human IL-2 sequence, or any combination of these substitutions. In various embodiments, the IL-2 variant has reduced/abolished binding to IL-2Rα compared to the native IL-2 polypeptide. In various embodiments, the IL-2 variant has decreased binding activity for the IL-2Rβγ receptors compared to the native IL-2 polypeptide. In various embodiments, the IL-2 variant has both reduced/abolished binding to IL-2Rα and modulated binding activity for the IL-2Rβγ receptors compared to the native IL-2 polypeptide. In various embodiments, the IL-2 variant is selected from the group of sequences set forth in SEQ ID NOS: 117-180.

In various embodiments, the active moiety of the PD1 Ab-IL-2 VitoKine is an IL-2 domain comprising the sequence of the mature human IL-2 polypeptide as set forth in SEQ ID NO: 116. In various embodiments, the IL-2 domain is an IL-2 variant (or mutant) comprising a sequence derived from the sequence of the mature human IL-2 polypeptide as set forth in SEQ ID NO: 116 comprising one or more amino acid substitution, deletion, or insertion. In various embodiments, the amino acid change is one or more amino acid substitutions at position 19, 65, 125 or 126 of SEQ ID NO: 116. In various embodiments, the amino acid change is the substitution of L to D or H or N or P or Q or R or S or Y at position 19, P to G or E or H or R or A or K or N or Q at position 65, C to I at position 125, Q to A or D or E or F or G or H or I or K or L or M or N or P or R or S or T or V or W or Y at position 126, of the mature human IL-2 sequence, or any combination of these substitutions. In various embodiments, the VitoKine construct will comprise an IL-2 moiety designed with reduced/abolished binding to IL-2Rα. In various embodiments, the IL-2 variant has decreased binding activity for IL-2Rβγ compared to the native IL-2 polypeptide. In various embodiments, the IL-2 variant has both reduced/abolished binding to IL-2Rα and altered binding activity for IL-2Rβγ compared to the native IL-2 polypeptide. In various embodiments, the IL-2 variant in the VitoKine construct can tune the IL-2 VitoKine intrinsic basal activity to achieve optimal antitumor efficacy while minimizing unwanted systematic toxicity for broadened therapeutic window. In various embodiments, the IL-2 domain is selected from the group of sequences set forth in SEQ ID NOS: 117-180.

In various embodiments, the concealing moiety domain is a cognate receptor/binding partner, or any binding partner identified for the IL-2. In various embodiments, the concealing moiety domain is an IL-2Rα extracellular domain with the sequence set forth in SEQ ID NO: 181 or a functional fragment thereof. In various embodiments, the IL-2Rα extracellular domain or a functional fragment thereof is an IL-2Rα sushi domain with the sequence set forth in SEQ ID NO: 182. In various embodiments, the concealing moiety domain is a variant (mutant) of IL-2RαSushi domain. In various embodiments, the amino acid change is one or more amino acid substitutions at position 36, 38, 42, or 43 of SEQ ID NO: 182. In various embodiments, the amino acid change is the substitution of R to A at position 36, K to E at position 38, L to G at position 42, Y to A at position 43. In various embodiments, the variant (mutant) of IL-2RαSushi domain is designed to facilitate dissociation and diffusion away after proteolytic cleavage. In various embodiments, the variant (mutant) of IL-2RαSushi domain is selected from the group of sequences set forth in SEQ ID NOS: 183-185.

In various embodiments, L1 linker and L2 linker of the PD1 Ab-IL-2 VitoKine constructs are both a protease cleavable peptide linker. In various embodiments, L1 of the PD1 Ab-IL-2 VitoKine constructs is a protease cleavable peptide linker and L2 is a non-cleavable peptide linker. In various embodiments, L1 of the PD1 Ab-IL-2 VitoKine constructs is a non-cleavable peptide linker and L2 is a protease cleavable peptide linker. In various embodiments, L1 linker and L2 linker of the PD1 Ab-IL-2 VitoKine constructs are both a protease non-cleavable peptide linker. In various embodiments, the non-cleavable linker is rich in G/S content (e.g., at least about 60%, 70%, 80%, 90%, or more of the amino acids in the linker are G or S). Each peptide linker sequence can be selected independently. In various embodiments, the protease cleavable linker is selected from the group of sequences set forth in SEQ ID NOS: 54-77. In various embodiments, the protease cleavable linker can have additional peptide spacer of variable length on the N-terminus of the cleavable linker or on the C-terminus of the cleavable linker or on both termini of the cleavable linker to improve accessibility for enzymatic cleavage. In various embodiments, the protease cleavable linker with peptide spacer of variable length on either the N-terminus or on the C-terminus or on both termini of the cleavable linker is selected from the group of sequences set forth in SEQ ID NOS: 78-94. In various embodiments, the non-cleavable linker is selected from the group of sequences set forth in SEQ ID NOS: 95-115. In various embodiments, the linker is either flexible or rigid and of a variety of lengths.

In various embodiments, the IL-2 domain (D2) and IL-2Rα domain (D3) of the VitoKine construct are placed at the C-terminus of the PD1 Ab domain (D1) as depicted in FIG. 1A. In various embodiments, the D2 and D3 domains of the VitoKine construct are placed at the N-terminus of the D1 domain as depicted in FIG. 1B.

In various embodiments, the PD1 blocking Ab, IL-2 domain and IL-2Rα domains of the PD1 Ab-IL-2 VitoKine construct can be monomer or dimer or a combination of dimer and monomer, such as PD1 blocking Ab is dimer and IL-2 domain and IL-2Rα domains are monomer.

In another aspect, the present disclosure provides a method for treating cancer or cancer metastasis in a subject, comprising administering a therapeutically effective amount of the pharmaceutical compositions of the invention to a subject in need thereof. In one embodiment, the subject is a human subject. In various embodiments, the cancer is selected from pancreatic cancer, gastric cancer, liver cancer, breast cancer, ovarian cancer, colorectal cancer, melanoma, leukemia, myelodysplastic syndrome, lung cancer, prostate cancer, brain cancer, bladder cancer, head-neck cancer, or rhabdomyosarcoma or any cancer.

In another aspect, the present disclosure provides a method for treating cancer or cancer metastasis in a subject, comprising administering a therapeutically effective amount of the pharmaceutical compositions of the invention in combination with a second therapy selected from the group consisting of: cytotoxic chemotherapy, immunotherapy, small molecule kinase inhibitor targeted therapy, surgery, radiation therapy, stem cell transplantation, cell therapies including chimeric antigen receptor (CAR)-T, CAR-NK, induced pluripotent stem cells (iPS) induced CAR-T or iPS induced CAR-NK and vaccine such as Bacille Calmette-Guerine (BCG). In various embodiments, the combination therapy may comprise administering to the subject a therapeutically effective amount of immunotherapy, including, but are not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, PD-L1, CD40, OX-40, CD137, GITR, LAG3, TIM-3, Siglec-7, Siglec-8, Siglec-9, Siglec-15 and VISTA; treatment using bispecific T cell engaging antibodies (BiTE®) such as blinatumomab: treatment involving administration of biological response modifiers such as IL-12, IL-21, GM-CSF, IFN-□□□IFN-β and IFN-γ; treatment using therapeutic vaccines such as sipuleucel-T; treatment using dendritic cell vaccines, or tumor antigen peptide vaccines; treatment using CAR-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR) agonists CpG and imiquimod; and treatment using vaccine such as BCG; wherein the combination therapy provides increased effector cell killing of tumor cells, i.e., a synergy exists between the VitoKine constructs and the immunotherapy when co-administered.

In another aspect, the disclosure provides uses of the pharmaceutical compositions of the invention for the preparation of a medicament for the treatment of cancer.

In another aspect, the present disclosure provides isolated nucleic acid molecules comprising a polynucleotide encoding of the pharmaceutical compositions of the present disclosure. In another aspect, the present disclosure provides vectors comprising the nucleic acids described herein. In various embodiments, the vector is an expression vector. In another aspect, the present disclosure provides isolated cells comprising the nucleic acids of the disclosure. In various embodiments, the cell is a host cell comprising the expression vector of the disclosure. In another aspect, methods of making the VitoKine constructs are provided by culturing the host cells under conditions promoting expression of the proteins or polypeptides.

In another aspect, the present disclosure provides a pharmaceutical composition comprising the isolated pharmaceutical compositions of the invention in admixture with a pharmaceutically acceptable carrier.

### BRIEF DESCRIPTION OF THE FIGURES

FIG. 1 depicts representative VitoKine construct formats. FIG. 1A depicts VitoKine construct with the D2 (active moiety domain) and D3 (concealing moiety domain) being placed at the C-terminus of the D1 (targeting domain). FIG. 1B depicts VitoKine construct with the D2 and D3 domains being placed at the N-terminus of the D1 domain.

FIG. 2 depicts the proposed activation mechanism for the PD1 Ab-IL-2 VitoKine constructs of the present invention. The exemplary VitoKine construct comprises two protease-cleavable linkers; protease 1 activation resulted from cleavage of L1 linker yields Active Form 1; protease 2 activation resulted from cleavage of L2 linker yields Active Form 2; activation by both proteases resulted from cleavage of L1 and L2 linkers yields Active Form 3. Following protease cleavage, the concealing moiety domain (D3) will release and diffuse away from the active moiety domain (D2). If the L1 linker is the only protease-cleavable linker, then Active Form 1 will be the sole activated format. Similarly, if L2 linker is the only protease-cleavable linker, then Active Form 2 will be the singular activated format.

FIG. 3A depicts representative PD1 Ab-IL-2 VitoKine construct of the present invention. A monomeric IL-2 or IL-2 variant as the active moiety domain (D2) is fused at its N-terminus to the C-terminus of a PD1 antibody heterodimeric Fc (D1) with L1 linker; the C-terminus of the IL-2 moiety is fused to the N-terminus of IL-2Rα or IL-2Rα variant as the concealing moiety domain (D3) with L2 linker. FIG. 3B depicts a representative PD1 Ab-IL-2 immunocytokine, which also acts as the non-VitoKine immunocytokine counterpart.

FIG. 4 depicts a comparison of the PD1 blocking activity between the Reference Antibody (P-0734) and pembrolizumab (PBL) biosimilar in a luciferase reporter assay. FIG. 4A and FIG. 4B depict dose-dependent increases in luminescence signal and fold induction, respectively. P-0734 and PBL biosimilar share the identical variable domains and have IgG1 and IgG4 isotypes, respectively.

FIG. 5 depicts (A) ELISA binding and (B-C) PD1 blockade activity of PD1 blocking antibodies, P-1148, P-1150, P-1151, and P-1153, compared to the Reference Antibody (P-0734), as tested in a luciferase reporter assay. FIG. 5B and FIG. 5C depict dose-dependent increases in luminescence signal and fold induction, respectively.

FIG. 6 depicts PD1 blockade activity of PD1 blocking antibodies, P-1127, P-1129, and P-1174, compared to the Reference Antibody (P-0734). They were tested in a luciferase reporter assay and the dose-dependent increases in luminescence signal are illustrated.

FIG. 7 depicts PD1 blockade activity of PD1 blocking antibodies, P-1175 and P-1181, compared to the Reference Antibody (P-0734), as tested in a luciferase reporter assay.

FIG. 7A and FIG. 7B depict dose-dependent increases in luminescence signal and fold induction, respectively.

FIG. 8 depicts PD1 blockade activity of PD1 blocking antibodies, P-1175, P-1176, P-1177, and P-1178, compared to the Reference Antibody (P-0734), as tested in a luciferase reporter assay. FIG. 8A and FIG. 8B depict dose-dependent increases in luminescence signal and fold induction, respectively.

FIG. 9 depicts PD1 blockade activity of PD1 blocking antibodies, P-1198, P-1199, and P-1201, compared to the Reference Antibody (P-0734), as tested in a luciferase reporter assay. FIG. 9A and FIG. 9B depict dose-dependent increases in luminescence signal and fold induction, respectively. A non-targeting germline antibody was included as the negative control.

FIG. 10 depicts PD1 blockade activity of PD1 blocking antibodies, P-1194, P-1201, and P-1238, compared to the Reference Antibody (P-0734), as tested in a luciferase reporter assay. FIG. 10A and FIG. 10B depict dose-dependent increases in luminescence signal and fold induction, respectively.

FIG. 11 depicts the binding of PD1 blocking antibodies, P-1174, P-1193, P-1198, P-1199, and P-1201 to PD1+ HEK293 cells, compared to the Reference Antibody (P-0734).

FIGS. 11A and 11C depict dose-dependent increases in percentage of positive cells, and FIGS. 11B and 11D depict dose-dependent increases in mean fluorescence intensity (MFI).

FIG. 12 depicts the ELISA binding of IL-2RαSushi variants, P-0751, P-0752, and P-0753, to IL-2. P-0757 comprises wild-type IL-2RαSushi and was included for comparison.

FIG. 13 depicts the activity assessment of Fc IL-2 VitoKines with either wild-type IL-2RαSushi as the D3 domain (P-0701) or IL-2RαSushi variants as the D3 domain (P-0754, P-0755, and P-0756). P-0704, an IL-2 P65R variant Fc fusion protein was included as a fully active IL-2 control. Activity was assessed by analyzing the induction of Ki67 expression on A) CD8+ T cells and B) NK cells of human PBMCs using flow cytometry.

FIG. 14 depicts ELISA binding of IL-2 variants to IL-2Rα. Each IL-2 variant harbors different amino acid substitution at position P65 (refer to Table 18 for details on molecular information). P-0531 and P-0689 serve as the wild-type IL-2 control in bivalent and monovalent forms, respectively.

FIG. 15 depicts the potency of IL-2 P65 variants in stimulating STAT5 phosphorylation in CD4+ Treg cells (refer to Table 18 for details on molecular information). P-0531 and P-0689 serve as the wild-type IL-2 control in bivalent and monovalent forms, respectively. Similarly, Benchmark (D) and Benchmark are bivalent and monovalent forms of IL-2 F42A/Y45A/L72G variant, respectively.

FIG. 16 depicts the activity of IL-2 P65 variants toward IL-2Rβγ by analyzing A) ELISA binding to a recombinantly expressed IL-2 receptor subunits β and γ complex, and B) the induction of Ki67 expression on CD8+ T cells in fresh human PBMCs using flow cytometry. Refer to Table 18 for details on molecular information of IL-2 variants. P-0531 and P-0689 serve as the wild-type IL-2 control in bivalent and monovalent forms, respectively.

FIG. 17 depicts the activity assessment of various surrogate mouse PD1 Ab-IL-2 VitoKines, P-0800, P-0830, P-0831, and P-0802, in comparison to P-0782, a non-VitoKine immunocytokine counterpart. This was done by analyzing the induction of Ki67 expression on A) CD8+ T cells and B) NK cells of human PBMCs. The four IL-2 antibody VitoKines differ only in the binding strength of their IL-2 moiety domains to IL-2Rα.

FIG. 18 depicts the activity assessment of IL-2 variants, P-0731, P-0759, and P-0761, harboring mutations that disrupt their interaction with IL-2Rβ. This evaluation was based on their effects on the induction of Ki67 expression on A) CD8+ T cells and B) NK cells of human PBMCs. All these IL-2 variants also possess the P65R mutation, which eliminates binding to IL-2Rα. P-0704 is utilized as a fully active IL-2 control.

FIG. 19 depicts the activity evaluation of IL-2 variants harboring mutations that interfere their binding to γc. This assessment was based on their effects on the induction of Ki67 expression on CD8+ T cells (A, C, & E) and NK cells (B, D, F) of human PBMCs. All these IL-2 variants also possess the P65R mutation, which eliminates IL-2 Ra binding. P-0704 functions as a fully active IL-2 control.

FIG. 20 depicts the activity assessment of the IL-2 variant in P-1247 harboring mutations targeting both IL-2Rβ and γc in comparison to P-1158, which contains only γc-interfering mutation. The evaluation was done by analyzing the effect on the induction of Ki67 expression on A) CD8+ T cells and B) NK cells of human PBMCs. P-0704 serves as an IL-2 full agonist control.

FIG. 21 depicts PD1 blockade activity of the PD1 blocking antibodies, P-1174, P-1238, and P-1271 in comparison to their respective PD1 Ab-IL-2 VitoKines, P-1197, P-1239, and P-1272, in a luciferase reporter assay. FIG. 21A and FIG. 21B depict dose-dependent increases in luminescence signal and fold induction, respectively.

FIG. 22 depicts the assessment of the intrinsic basal IL-2 activity of PD1 Ab-IL-2 VitoKines P-0872, P-1197, and P-1272 in comparison to their corresponding non-VitoKine immunocytokine counterparts, P-0879 and P-1271. The activity evaluation was based on analyzing the induction of proliferation marker Ki67 on CD8+ T cells (A &C) and NK cells (B &D) of human PBMCs. The three VitoKines differ only in the D1 domain composition, namely they contain distinct PD1 blocking antibodies. P-1174, the component PD1 antibody of P-1197, was included as a negative control for the assay.

FIG. 23 depicts the protease cleavage and activation of PD1 Ab-IL-2 VitoKines. The figure includes A) a reduced SDS-PAGE gel showing both the intact and active forms of P-1272, along with demonstrations of dose-dependent induction of Ki67 expression on CD8+ T cells for B) VitoKine P-1272 versus its non-VitoKine counterpart P-1273, C) VitoKine P-0831 versus its non-VitoKine counterpart P-0838, and D) VitoKine P-1345 versus its non-VitoKine counterpart P-0838.

FIG. 24 depicts the serum concentrations of mouse PD1 Ab-IL-2 VitoKine (P-0831) and its non-VitoKine immunocytokine equivalent, P-0838, following a single intraperitoneal injection in C57B/L6 mice. Blood was collected from mice at multiple time points post-dosing and ELISA assays were used to determine the compounds' serum levels.

FIG. 25 depicts the dose and time-dependent effects of a single dose of P-0831, a mouse PD1 Ab-IL-2 VitoKine, on the expansion of A) CD8+ T cells, B) granzyme B+CD8+ T cells, C) NK cells, and D) granzyme B+NK cells in peripheral blood in C57B/L6 mice. P-0838, its non-VitoKine immunocytokine equivalent, was included for comparison. Blood was collected on Days 0, 3, 5, 7, and 10 for lymphocyte phenotyping by FACS analysis. The Data is expressed as mean±SEM.

FIG. 26 depicts the effects of a single dose of P-0831, a mouse PD1 Ab-IL-2 VitoKine, on (A) the dose-dependent increases in the serum levels of the inflammatory marker IFNγ, and (B) changes in body weight across different dosage levels in naïve C57BL/6 mice. P-0838, its non-VitoKine immunocytokine equivalent, was included for comparison. Additionally, the vehicle (PBS) and its component mouse PD1 antibody, P-0722, were used as negative controls.

FIG. 27 depicts the antitumor effects of P-0831, a mouse PD1 Ab-IL-2 VitoKine, in an established MC38 murine colon carcinoma model after two doses administered once every 10 days (Q10D). The growth curve of MC38 tumors in individual mice is presented for A) P-0831 at 3 mg/kg, B) P-0831 at 6 mg/kg, C) P-0831 at 9 mg/kg, and D) P-0838, its non-VitoKine immunocytokine equivalent, at 1 mg/kg. For comparison, the mean tumor volume ±standard error of the mean (SEM) over time for the vehicle group plotted with a dotted line. The mean tumor volume ±SEM over time for each treatment group is illustrated in FIG. 27E, and the change in body weight over time for each treatment group is shown in FIG. 27F.

FIG. 28 depicts the immunohistochemistry (IHC) analysis of the effect of P-0831, a mouse PD1 Ab-IL-2 VitoKine, in the tumor tissues isolated 5 days post treatment at a dosage of 6 mg/kg. The tissue sections were fixed in 10% formalin, paraffin-embedded, processed, and stained with antibodies by HistoWiz to evaluate immune cells in the tumor tissue. For comparison, P-0722, its component mouse PD1 antibody, was included at a dosage of 6 mg/kg, and P-0838, its non-VitoKine immunocytokine counterpart, at a dosage of 1 mg/kg.

FIG. 29 depicts P-0831's ex vivo activity in human PBMCs and in vivo anti-tumor efficacy in mice with established CT26 murine tumors, compared to its non-cleavable VitoKine counterpart, P-0877. The in vitro assessment measures the induction of proliferation marker Ki67 on A) CD8+ T cells, and B) NK cells of fresh human PBMCs. The in vivo analysis include C) mean tumor volume ±SEM and D) changes in body weight over time for each treatment group in CT26 murine model following two Q12D doses of 10 mg/kg. P-0879 was used as the fully active IL-2 immunocytokine control in human PBMC assay. P-0722, P-0831's component mouse PD1 antibody, dosed at 10 mg/kg was included as a control in the tumor model.

FIG. 30 depicts P-0831's in vivo anti-tumor efficacy in mice with established CT26 murine tumors, compared to its non-targeting VitoKine counterpart, P-0871 and its component mouse PD1 antibody, P-0722. Mean tumor volume ±SEM was plotted as a function of time for each treatment group in CT26 murine model following two Q12D doses of 10 mg/kg.

FIG. 31 depicts the activity assessment of mouse PD1 Ab-IL-2 immunocytokines, P-0782, P-0783, and P-0786, by analyzing their effects on A) the induction of Ki67 expression on human CD8+ T cells, B) the induction of Ki67 expression on human NK cells, and C) the proliferation of mouse CTLL-2 cells.

FIG. 32 depicts the dose-dependent and temporal pharmacodynamic effects of several mouse PD1 Ab-IL-2 immunocytokines. This includes A) the expansion of peripheral CD8+ T cells, B) the expansion of peripheral NK cells, and C) changes in body weight following a single dose of 2 mg/kg in C57B/L6 mice. Blood was collected on Days 0, 3, 5, 7, and 10 for lymphocyte phenotyping by FACS analysis. Data are expressed as mean±SEM.

FIG. 33 depicts the antitumor efficacy of several mouse PD1 Ab-IL-2 immunocytokines in an established MC38 murine colon carcinoma model following two Q12D treatments at 0.5 mg/kg. The mean tumor volume ±SEM over time for each treatment group is illustrated in FIG. 33A, and the individual tumor volumes in mice on Day 26 following the first dose are shown in FIG. 33B.

FIG. 34 depicts the antitumor efficacy of two mouse PD1 Ab-IL-2 immunocytokines, P-0783 and P-0786, in an established MC38 murine colon carcinoma model following two Q10D doses at 0.3 mg/kg. The mean tumor volume ±SEM over time for each treatment group is illustrated in FIG. 34A. The individual growth curves of the subcutaneous MC38 tumors in mice are illustrated for B) P-0783 and C) P-0786.

FIG. 35 depicts the antitumor efficacy of P-0786, a mouse PD1 Ab-IL-2 immunocytokine, in an established MC38 murine colon carcinoma model following two Q10D treatments at 1 mg/kg. This includes A) the mean tumor volume ±SEM and B) changes in body weight over time for each treatment group. The component mouse PD1 Antibody, P-0722 dosed at 9 mg/kg, was included for comparison.

FIG. 36 depicts the dose-dependent antitumor efficacy of P-0786, a mouse PD1 Ab-IL-2 immunocytokine, in an established MC38 murine colon carcinoma model following two Q14D treatments at varying dosing levels. The mean tumor volume ±SEM over time for each treatment group was illustrated in FIG. 36A. The individual tumor growth curves are illustrated for B) 0.03 mg/kg, C) 0.1 mg/kg, C) 0.3 mg/kg, and E) 1 mg/kg dosages. The mean tumor volume ±SEM over time for the vehicle group (represented as a dotted line) is included for comparison.

FIG. 37 depicts the absence of tumor recurrence after rechallenge implantation of MC38 colon carcinoma cells in P-0786-treated tumor-free mice. This is contrasted with the successful regrowth of the same type of tumor in age-matched naïve mice as a control.

FIG. 38 depicts the efficacy of P-0786 as a single agent in inhibiting tumor growth in two additional subcutaneous syngeneic tumor models. These models include A) the CT26 murine colon carcinoma tumor model and B) the B16F10 murine melanoma tumor model.

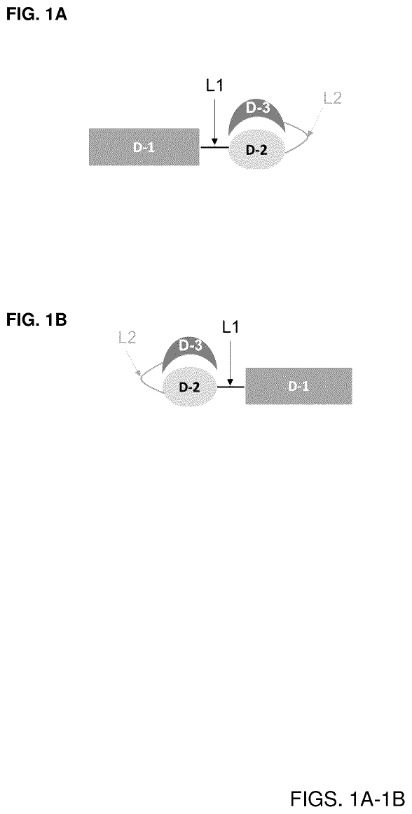

### MODE(S) FOR CARRYING OUT THE DISCLOSURE

In one aspect, the present disclosure provides PD1 Ab-IL-2 VitoKine constructs comprise 3 domains: an optimized PD1 blocking antibody as the TIL-targeting moiety, an IL-2 variant as the active moiety domain, and an IL-2 Ra sushi variant as the concealing moiety domain. Importantly, the IL-2 Ra sushi variant domain is capable of concealing or attenuating the functional activity of IL-2 domain until activated at the intended site of therapy.

The PD1 blocking antibody guides the VitoKine to the TILs in the tumor microenvironment and restrict the activation of the VitoKine locally to improve the therapeutic index. In various embodiments, the PD1 blocking antibodies were optimized through modifications in the variable domains of pembrolizumab. In various embodiments, the modifications involved germline sequence substitutions of the CDR residues, germline sequence substitutions of the framework residues, and adoption of the VH3 human germline family sequence as the acceptor framework. In various embodiments, these modifications were implemented individually or in combination to develop optimized PD1 blocking antibodies. In various embodiments, these optimized PD1 blocking antibodies exhibit a high binding affinity to PD1, function to inhibit PD1 with equal or comparable potency as pembrolizumab, have a higher sequence similarity score to its closest human germline sequence, resulting in an improved degree of humanness compared to pembrolizumab, and are predicted to have lower hydrophobicity, leading to lowered aggregation propensity. In various embodiments, PD1 Ab-IL-2 VitoKine constructs based on these optimized PD1 blocking antibodies have enhanced developability properties.

In various embodiments, the IL-2 domain is the active moiety but remains inert until activated locally by proteases that are upregulated in diseased tissues; this will limit binding of the active moiety to the receptors in the peripheral or on the cell-surface of non-diseased cells or tissue to prevent over-activation of the pathway and reduce undesirable “on-target” “off tissue” toxicity. The improved safety profile of the VitoKines may permit human dose levels within the effective range of a PD1 antibody. Additionally, the inertness of the VitoKine active moiety prior to protease activation will significantly decrease the potential antigen sink, and thus, prolong the in vivo half-life and result in improved biodistribution, bioavailability and efficacy at intended sites of therapy.

In various embodiments, the integration of a potency-attenuated IL-2 variant as the active moiety domain (such IL-2 variant achieved by disrupting IL-2Rβγ interaction) can further fine-tune the intrinsic basal activity and post-activation activity of the VitoKine. In various embodiments, such VitoKine with potency attenuated IL-2 variant as the active moiety domain may additionally expand the therapeutic index.

In various embodiments, the unique and non-signaling a-subunit of receptors of IL-2 is used as the concealing moiety domain via a protease-cleavable linker to reversibly conceal the cytokine activity. The concealing a-subunit may be preferred to dissociate away after protease cleavage of the linker. As a result, amino acid modifications of the a-receptor to modulate the binding affinity to IL-2 may be beneficial.

In various embodiments, in the PD1 Ab-IL-2 VitoKine constructs, the three domains are linked using two linkers with variable length and rigidity and are optionally coupled with protease-cleavable sequences. These protease-cleavable sequences are peptide substrates of specific protease subtypes with elevated or dysregulated expression in the disease sites, thus allowing for a functional IL-2 domain to be revealed or released at the site of disease. The linker length and composition were fine-tuned to ensure optimal concealment of the IL-2 domain from accessing its receptors, thus minimizing systemic engagement. Meanwhile, the stability of the VitoKine construct in the blood circulation was maintained while allowing efficient cleavage upon encountering specific proteases at the intended site of therapy.

In another aspect, the present disclosure provides novel PD1-targeted IL-2 immunocytokines that aim to target an activity-modulated IL-2 domain directly to tumor-infiltrating lymphocytes. In various embodiments, the PD1 blocking antibodies were optimized through modifications in the variable domains of pembrolizumab. In various embodiments, targeted IL-2 immunocytokines based on the optimized PD1 blocking antibodies are predicted to have enhanced developability properties.

In various embodiments, the activity-modulated IL-2 domain (monomeric) is fused to the C-terminus of heterodimeric PD1 antibody heavy chains. In various embodiments, the IL-2 domain in PD1-targeted IL-2 immunocytokine is IL-2Rβγ-selective and potency attenuated. In various embodiments, IL-2 potency attenuation facilitates establishing a stoichiometric balance between the cytokine and antibody components, helps to alleviate pathway over-activation, and mitigates antigen sink and target-mediated deposition. In various embodiments, use of a potency-attenuated IL-2 variant (such variant having impaired interaction with γc) in the PD1 targeted IL-2 immunocytokine could offer additional benefits in mitigating antigen sink and in turn result in an extend in vivo half-life likely because of the impact of γc receptor in the signaling cascade leading to cell expansion.

### DEFINITIONS

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those commonly used and well known in the art. The methods and techniques of the present invention are generally performed according to conventional methods well known in the art and as described in various general and more specific references that are cited and discussed throughout the present specification unless otherwise indicated. See, e.g., Green and Sambrook, Molecular Cloning: A Laboratory Manual, 4th ed., Cold Spring Harbor Laboratory Press, Cold Spring Harbor, N.Y. (2012), incorporated herein by reference. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those commonly used and well known in the art. Standard techniques are used for chemical syntheses, chemical analyses, pharmaceutical preparation, formulation, and delivery, and treatment of subjects.

The terms “polypeptide”, “peptide” and “protein” are used interchangeably herein to refer to a polymer of amino acid residues. In various embodiments, “peptides”, “polypeptides”, and “proteins” are chains of amino acids whose alpha carbons are linked through peptide bonds. The terminal amino acid at one end of the chain (amino terminal) therefore has a free amino group, while the terminal amino acid at the other end of the chain (carboxy terminal) has a free carboxyl group. As used herein, the term “amino terminus” (abbreviated N-terminus) refers to the free a-amino group on an amino acid at the amino terminal of a peptide or to the a-amino group (amino group when participating in a peptide bond) of an amino acid at any other location within the peptide. Similarly, the term “carboxy terminus” (abbreviated C-terminus) refers to the free carboxyl group on the carboxy terminus of a peptide or the carboxyl group of an amino acid at any other location within the peptide. Peptides also include essentially any polyamino acid including, but not limited to, peptide mimetics such as amino acids joined by an ether as opposed to an amide bond.

Polypeptides of the disclosure include polypeptides that have been modified in any way and for any reason, for example, to: (1) reduce susceptibility to proteolysis, (2) reduce susceptibility to oxidation, (3) alter binding affinity for forming protein complexes, (4) alter binding affinities, and (5) confer or modify other physicochemical or functional properties.

An amino acid “substitution” as used herein refers to the replacement in a polypeptide of one amino acid at a particular position in a parent polypeptide sequence with a different amino acid. Amino acid substitutions can be generated using genetic or chemical methods well known in the art. For example, single or multiple amino acid substitutions (e.g., conservative amino acid substitutions) may be made in the naturally-occurring sequence (e.g., in the portion of the polypeptide outside the domain(s) forming intermolecular contacts). A “conservative amino acid substitution” refers to the substitution in a polypeptide of an amino acid with a functionally similar amino acid. The following six groups each contain amino acids that are conservative substitutions for one another:
1) Alanine (A), Serine (S), and Threonine (T)
2) Aspartic acid (D) and Glutamic acid (E)
3) Asparagine (N) and Glutamine (Q)
4) Arginine (R) and Lysine (K)
5) Isoleucine (I), Leucine (L), Methionine (M), and Valine (V)
6) Phenylalanine (F), Tyrosine (Y), and Tryptophan (W)

A “non-conservative amino acid substitution” refers to the substitution of a member of one of these classes for a member from another class. In making such changes, according to various embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (see, for example, Kyte et al., 1982, J. Mol. Biol. 157:105-131). It is known that certain amino acids may be substituted for other amino acids having a similar hydropathic index or score and still retain a similar biological activity. In making changes based upon the hydropathic index, in various embodiments, the substitution of amino acids whose hydropathic indices are within +2 is included. In various embodiments, those that are within +1 are included, and in various embodiments, those within +0.5 are included.

It is also understood in the art that the substitution of like amino acids can be made effectively on the basis of hydrophilicity, particularly where the biologically functional protein or peptide thereby created is intended for use in immunological embodiments, as disclosed herein. In various embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0.±0.1); glutamate (+3.0.±0.1); serine (+0.3); asparagine (+0.2); glutamine (+0.2); glycine (0); threonine (−0.4); proline (−0.5.±0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in various embodiments, the substitution of amino acids whose hydrophilicity values are within +2 is included, in various embodiments, those that are within +1 are included, and in various embodiments, those within +0.5 are included.

Exemplary amino acid substitutions are set forth in Table 1.

TABLE 1

| Original | Exemplary | Preferred |
|---|---|---|
| Residues | Substitutions | Substitutions |
| Ala | Val, Leu, Ile | Val |
| Arg | Lys, Gln, Asn | Lys |
| Asn | Gln | |
| Asp | Glu | |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, 1,4 Diamino-butyric | Arg |
| | Acid, Gln, Asn | |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | |
| Trp | Tyr, Phe | Tyr |
| Tyr | Trp, Phe, Thr, Ser | Phe |
| Val | Ile, Met, Leu, Phe, | Leu |
| | Ala, Norleucine | |

A skilled artisan will be able to determine suitable variants of polypeptides as set forth herein using well-known techniques. In various embodiments, one skilled in the art may identify suitable areas of the molecule that may be changed without destroying activity by targeting regions not believed to be important for activity. In other embodiments, the skilled artisan can identify residues and portions of the molecules that are conserved among similar polypeptides. In further embodiments, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, the skilled artisan can predict the importance of amino acid residues in a polypeptide that correspond to amino acid residues important for activity or structure in similar polypeptides. One skilled in the art may opt for chemically similar amino acid substitutions for such predicted important amino acid residues.

One skilled in the art can also analyze the three-dimensional structure and amino acid sequence in relation to that structure in similar polypeptides. In view of such information, one skilled in the art may predict the alignment of amino acid residues of a polypeptide with respect to its three-dimensional structure. In various embodiments, one skilled in the art may choose to not make radical changes to amino acid residues predicted to be on the surface of the polypeptide, since such residues may be involved in important interactions with other molecules. Moreover, one skilled in the art may generate test variants containing a single amino acid substitution at each desired amino acid residue. The variants can then be screened using activity assays known to those skilled in the art. Such variants could be used to gather information about suitable variants. For example, if one discovered that a change to a particular amino acid residue resulted in destroyed, undesirably reduced, or unsuitable activity, variants with such a change can be avoided. In other words, based on information gathered from such routine experiments, one skilled in the art can readily determine the amino acids where further substitutions should be avoided either alone or in combination with other mutations.

The term “polypeptide fragment” and “truncated polypeptide” as used herein refers to a polypeptide that has an amino-terminal and/or carboxy-terminal deletion as compared to a corresponding full-length protein. In various embodiments, fragments can be, e.g., at least 5, at least 10, at least 25, at least 50, at least 100, at least 150, at least 200, at least 250, at least 300, at least 350, at least 400, at least 450, at least 500, at least 600, at least 700, at least 800, at least 900 or at least 1000 amino acids in length. In various embodiments, fragments can also be, e.g., at most 1000, at most 900, at most 800, at most 700, at most 600, at most 500, at most 450, at most 400, at most 350, at most 300, at most 250, at most 200, at most 150, at most 100, at most 50, at most 25, at most 10, or at most 5 amino acids in length. A fragment can further comprise, at either or both of its ends, one or more additional amino acids, for example, a sequence of amino acids from a different naturally-occurring protein (e.g., an Fc or leucine zipper domain) or an artificial amino acid sequence (e.g., an artificial linker sequence).

The terms “polypeptide variant”, “hybrid polypeptide” and “polypeptide mutant” as used herein refers to a polypeptide that comprises an amino acid sequence wherein one or more amino acid residues are inserted into, deleted from and/or substituted into the amino acid sequence relative to another polypeptide sequence. In various embodiments, the number of amino acid residues to be inserted, deleted, or substituted can be, e.g., at least 1, at least 2, at least 3, at least 4, at least 5, at least 10, at least 25, at least 50, at least 75, at least 100, at least 125, at least 150, at least 175, at least 200, at least 225, at least 250, at least 275, at least 300, at least 350, at least 400, at least 450 or at least 500 amino acids in length. Hybrids of the present disclosure include fusion proteins.

A “derivative” of a polypeptide is a polypeptide that has been chemically modified, e.g., conjugation to another chemical moiety such as, for example, polyethylene glycol, albumin (e.g., human serum albumin), phosphorylation, and glycosylation.

The term “% sequence identity” is used interchangeably herein with the term “% identity” and refers to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm and means that a given sequence is at least 80% identical to another length of another sequence. In various embodiments, the % identity is selected from, e.g., at least 60%, at least 65%, at least 70%, at least 75%, at least 80%, at least 85%, at least 90%, at least 95%, or at least 99% or more sequence identity to a given sequence. In various embodiments, the % identity is in the range of, e.g., about 60% to about 70%, about 70% to about 80%, about 80% to about 85%, about 85% to about 90%, about 90% to about 95%, or about 95% to about 99%.

The term “% sequence homology” is used interchangeably herein with the term “% homology” and refers to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence. In various embodiments, the % homology is selected from, e.g., at least 60%, at least 65%, at least 70%, at least 75%, at least 80%, at least 85%, at least 90%, at least 95%, or at least 99% or more sequence homology to a given sequence. In various embodiments, the % homology is in the range of, e.g., about 60% to about 70%, about 70% to about 80%, about 80% to about 85%, about 85% to about 90%, about 90% to about 95%, or about 95% to about 99%.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at the NCBI website. See also Altschul et al., J. Mol. Biol. 215:403-10, 1990 (with special reference to the published default setting, i.e., parameters w=4, t=17) and Altschul et al., Nucleic Acids Res., 25:3389-3402, 1997. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix.

In addition to calculating percent sequence identity, the BLAST algorithm also performs a statistical analysis of the similarity between two sequences (see, e.g., Karlin & Altschul, Proc. Natl. Acad. Sci. USA, 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is, e.g., less than about 0.1, less than about 0.01, or less than about 0.001.

The term “modification” as used herein refers to any manipulation of the peptide backbone (e.g., amino acid sequence) or the post-translational modifications (e.g., glycosylation) of a polypeptide.

The term “knob-into-hole modification” as used herein refers to a modification within the interface between two immunoglobulin heavy chains in the CH3 domain. In one embodiment, the “knob-into-hole modification” comprises the amino acid substitution T366W and optionally the amino acid substitution S354C in one of the antibody heavy chains, and the amino acid substitutions T366S, L368A, Y407V and optionally Y349C in the other one of the antibody heavy chains. The knob-into-hole technology is described, e.g., in U.S. Pat. Nos. 5,731,168; 7,695,936; Ridgway et al., Prot Eng 9, 617-621 (1996) and Carter, J Immunol Meth 248, 7-15 (2001).

The term “bioactivatable drug” or “VitoKine” as used herein means a compound that is a drug precursor which, following administration to a subject, releases the drug in vivo via some chemical or physiological process such that the bioactivatable drug is converted into a product that is active to the target tissues. A bioactivatable drug is any compound that undergoes bioactivation before exhibiting its pharmacological effects. Bioactivatable drugs can thus be viewed as drugs containing specialized non-toxic protective groups used in a transient manner to alter or to eliminate undesirable properties in the parent molecule.

The term “immunoconjugate” or “fusion protein” as used herein refers to a molecule comprising an antibody or antigen-binding fragment thereof conjugated (or linked) directly or indirectly to an effector molecule. The effector molecule can be a detectable label, an immunotoxin, cytokine, chemokine, therapeutic agent, or chemotherapeutic agent. The antibody or antigen-binding fragment thereof may be conjugated to an effector molecule via a peptide linker. An immunoconjugate and/or fusion protein retains the immunoreactivity of the antibody or antigen-binding fragment, e.g., the antibody or antigen-binding fragment has approximately the same, or only slightly reduced, ability to bind the antigen after conjugation as before conjugation. As used herein, an immunoconjugate may also be referred to as an antibody drug conjugate (ADC). Because immunoconjugates and/or fusion proteins are originally prepared from two molecules with separate functionalities, such as an antibody and an effector molecule, they are also sometimes referred to as “chimeric molecules.”

“Linker” refers to a molecule that joins two other molecules, either covalently, or through ionic, van der Waals or hydrogen bonds, e.g., a nucleic acid molecule that hybridizes to one complementary sequence at the 5′ end and to another complementary sequence at the 3′ end, thus joining two non-complementary sequences. A “cleavable linker” refers to a linker that can be degraded, digested, or otherwise severed to separate the two components connected by the cleavable linker. Cleavable linkers are generally cleaved by enzymes, typically peptidases, proteases, nucleases, lipases, and the like. Cleavable linkers may also be cleaved by environmental cues, such as, for example, changes in temperature, pH, salt concentration, etc.

The term “peptide linker” as used herein refers to a peptide comprising one or more amino acids, typically about 1-30 amino acids. Peptide linkers are known in the art or are described herein. Suitable, non-immunogenic linker peptides include, for example, (G₄S)ₙ, (SG₄)ₙ or G₄(SG₄)ₙ peptide linkers. “n” is generally a number between 1 and 10, typically between 2 and 4.

“Pharmaceutical composition” refers to a composition suitable for pharmaceutical use in an animal. A pharmaceutical composition comprises a pharmacologically effective amount of an active agent and a pharmaceutically acceptable carrier. “Pharmacologically effective amount” refers to that amount of an agent effective to produce the intended pharmacological result. “Pharmaceutically acceptable carrier” refers to any of the standard pharmaceutical carriers, vehicles, buffers, and excipients, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 21st Ed. 2005, Mack Publishing Co, Easton. A “pharmaceutically acceptable salt” is a salt that can be formulated into a compound for pharmaceutical use including, e.g., metal salts (sodium, potassium, magnesium, calcium, etc.) and salts of ammonia or organic amines.

As used herein, “treatment” (and grammatical variations thereof such as “treat” or “treating”) refers to clinical intervention in an attempt to alter the natural course of a disease in the individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. As used herein, to “alleviate” a disease, disorder or condition means reducing the severity and/or occurrence frequency of the symptoms of the disease, disorder, or condition. Further, references herein to “treatment” include references to curative, palliative and prophylactic treatment.

The term “effective amount” or “therapeutically effective amount” as used herein refers to an amount of a compound or composition sufficient to treat a specified disorder, condition or disease such as ameliorate, palliate, lessen, and/or delay one or more of its symptoms. In reference to cancers or other unwanted cell proliferation, an effective amount comprises an amount sufficient to: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. An effective amount can be administered in one or more administrations.

The phrase “administering” or “cause to be administered” refers to the actions taken by a medical professional (e.g., a physician), or a person controlling medical care of a patient, that control and/or permit the administration of the agent(s)/compound(s) at issue to the patient. Causing to be administered can involve diagnosis and/or determination of an appropriate therapeutic regimen, and/or prescribing particular agent(s)/compounds for a patient. Such prescribing can include, for example, drafting a prescription form, annotating a medical record, and the like. Where administration is described herein, “causing to be administered” is also contemplated.

The terms “patient,” “individual,” and “subject” may be used interchangeably and refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In various embodiments, the patient can be a human (e.g., adult male, adult female, adolescent male, adolescent female, male child, female child) under the care of a physician or other health worker in a hospital, psychiatric care facility, as an outpatient, or other clinical context. In various embodiments, the patient may be an immunocompromised patient or a patient with a weakened immune system including, but not limited to patients having primary immune deficiency, AIDS; cancer and transplant patients who are taking certain immunosuppressive drugs; and those with inherited diseases that affect the immune system (e.g., congenital agammaglobulinemia, congenital IgA deficiency). In various embodiments, the patient has an immunogenic cancer, including, but not limited to bladder cancer, lung cancer, melanoma, and other cancers reported to have a high rate of mutations (Lawrence et al., Nature, 499(7457): 214-218, 2013).

The term “immunotherapy” refers to cancer treatments which include, but are not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, PD1, PDL-1, CD40, OX-40, CD137, GITR, LAG3, TIM-3, SIRPα, CD47, GITR, ICOS, CD27, Siglec 7, Siglec 8, Siglec 9, Siglec 15, VISTA, CD276, CD272, TIM-3, and B7-H4; treatment using bispecific T cell engaging antibodies (BiTE®) such as blinatumomab: treatment involving administration of biological response modifiers such as IL-2, IL-4, IL-7, IL-10, IL-12, IL-15, IL-21, IL-22, GM-CSF, IFN-□□□IFN-β, IFN-γ, TGF-β antagonist or TGF-β trap; treatment using therapeutic vaccines such as sipuleucel-T; treatment using therapeutic virus, including, but not limited to oncolytic virus such as T-vec; treatment using dendritic cell vaccines, or tumor antigen peptide or neoantigen vaccines; treatment using NK cells; treatment using chimeric antigen receptor (CAR)-T cells; treatment using CAR-NK cells; treatment using DC or T cells; treatment using iPS induced-NK cells; treatment using iPS induced-T cells; treatment using vaccine such as Bacille Calmette-Guerine (BCG); treatment using tumor infiltrating lymphocytes (TILs); treatment using adoptively transferred anti-tumor T cells (ex vivo expanded and/or TCR-T cells); treatment using TALL-104 cells; and treatment using immunostimulatory agents such as Toll-like receptor (TLR) agonists CpG, TLR7,TLR8, TLR9, and imiquimod.

“Resistant or refractory cancer” refers to tumor cells or cancer that do not respond to previous anti-cancer therapy including, e.g., chemotherapy, surgery, radiation therapy, stem cell transplantation, and immunotherapy. Tumor cells can be resistant or refractory at the beginning of treatment, or they may become resistant or refractory during treatment. Refractory tumor cells include tumors that do not respond at the onset of treatment or respond initially for a short period but fail to respond to treatment. Refractory tumor cells also include tumors that respond to treatment with anticancer therapy but fail to respond to subsequent rounds of therapies. For purposes of this invention, refractory tumor cells also encompass tumors that appear to be inhibited by treatment with anticancer therapy but recur up to five years, sometimes up to ten years or longer after treatment is discontinued. The anticancer therapy can employ chemotherapeutic agents alone, radiation alone, targeted therapy alone, surgery alone, or combinations thereof. For ease of description and not limitation, it will be understood that the refractory tumor cells are interchangeable with resistant tumor.

The term “neoantigen” refers to, e.g., cell surface antigens to which the immune system has not previously been exposed, especially one that arises by alteration of host antigens by radiation, chemotherapy, viral infection, neoplastic transformation/mutation, drug metabolism, etc., selectively expressed by cancer cells or over-expressed in cancer cells relative to most normal cells.

The term “antibody” as used herein is used in the broadest sense and encompasses various antibody structures (IgG1, 2, 3, or 4, IgM, IgA, IgE) including but not limited to monoclonal antibodies, polyclonal antibodies, multi-specific antibodies (e.g., bispecific or bifunctional antibodies), and antibody fragments so long as they exhibit the desired antigen-binding activity.

The term “antibody fragment” as used herein refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2, diabodies, linear antibodies, single-chain antibody molecules (e.g., scFv), and single-domain antibodies.

The term “Fab fragment” as used herein refers to an immunoglobulin fragment comprising a VL domain and a constant domain of a light chain (CL), and a VH domain and a first constant domain (CH1) of a heavy chain.

The terms “variable region” or “variable domain” as used herein refers to the domain of an immunoglobulin or antibody heavy or light chain that is generally involved in binding the immunoglobulin or antibody to antigen. The variable domains of the heavy chain and light chain (VH and VL, respectively) of an immunoglobulin or antibody generally have similar structures, with each domain comprising four conserved framework regions (FRs) and three complementarity-determining regions (CDRs).

The term “complementarity determining regions” or “CDRs” contain the antigen-contacting residues (“antigen contacts”). Generally, antibodies comprise six CDRs: three in the VH (CDR-H1, CDR-H2, CDR-H3), and three in the VL (CDR-L1, CDR-L2, CDR-L3). CDRs occurring at amino acid residues 24-34 (CDR-L1), 50-56 (CDR-L2), 89-97 (CDR-L3), 31-35b (CDR-H1), 50-65 (CDR-H2), and 95-102 (CDR-H3) (Kabat et al., Sequences of Proteins of Immunological Interest, 5th Ed. Public Health Service, National Institutes of Health, Bethesda, MD (1991)). Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs)

“Single-chain antibodies” are Fv molecules in which the heavy and light chain variable regions have been connected by a flexible linker to form a single polypeptide chain, which forms an antigen binding region. Single chain antibodies are discussed in detail in International Patent Application Publication No. WO 88/01649, U.S. Pat. Nos. 4,946,778 and 5,260,203, the disclosures of which are incorporated by reference.

A “human immunoglobulin” as used herein is one which possesses an amino acid sequence which corresponds to that of an immunoglobulin produced by a human or a human cell or derived from a non-human source that utilizes human immunoglobulin repertoires or other human immunoglobulin-encoding sequences. This definition of a human immunoglobulin specifically excludes a humanized immunoglobulin comprising non-human antigen-binding residues.

The term “humanized antibody” as used herein refers to an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework, and such substitutions are herein referred to as back-mutations. Humanized or other monoclonal antibodies can have additional conservative amino acid substitutions which have substantially no effect on antigen binding or other immunoglobulin functions.

The term “Fc domain” or “Fc region” as used herein is used to define a C-terminal region of an immunoglobulin heavy chain that contains at least a portion of the constant region. The term includes native sequence Fc regions and variant Fc regions. An IgG Fc region comprises an IgG CH2 and an IgG CH3 domain. The CH3 region herein may be a native sequence CH3 domain or a variant CH3 domain (e.g., a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote heterodimerization of two non-identical immunoglobulin heavy chains as herein described. Unless otherwise specified herein, numbering of amino acid residues in the Fc region or constant region is according to the EU numbering system.

The term “effector functions” as used herein refers to those biological activities attributable to the Fc region of an immunoglobulin, which vary with the immunoglobulin isotype. Examples of immunoglobulin effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g., B cell receptor), and B cell activation.

As used herein, “specific binding” is meant that the binding is selective for the antigen and can be discriminated from unwanted or non-specific interactions. The ability of an immunoglobulin to bind to a specific antigen can be measured either through an enzyme-linked immunosorbent assay (ELISA) or other techniques familiar to one of skill in the art, e.g., surface plasmon resonance (SPR) technique.

The terms “affinity” or “binding affinity” as used herein refers to the strength of the sum total of non-covalent interactions between a single binding site of a molecule (e.g., an antibody) and its binding partner (e.g., an antigen). The affinity of a molecule X for its partner Y can generally be represented by the dissociation constant (KD), which is the ratio of dissociation and association rate constants (koff and kon, respectively). A particular method for measuring affinity is SPR.

The term “immunogenicity” as used herein refers to the ability of an antibody or antigen binding fragment to elicit an immune response (humoral or cellular) when administered to a recipient and includes, for example, the human anti-mouse antibody (HAMA) response. A HAMA response is initiated when T-cells from a subject make an immune response to the administered antibody. The T-cells then recruit B-cells to generate specific “anti-antibody” antibodies.

The term “immune cell” as used herein means any cell of hematopoietic lineage involved in regulating an immune response against an antigen (e.g., an autoantigen). In various embodiments, an immune cell is, e.g., a T cell, a B cell, a dendritic cell, a monocyte, a natural killer cell, a macrophage, Langerhan's cells, or Kuffer cells.

The term “reduced binding”, as used herein refers to a decrease in affinity for the respective interaction, as measured for example by SPR. Conversely, “increased binding” refers to an increase in binding affinity for the respective interaction.

The term “polymer” as used herein generally includes, but is not limited to, homopolymers; copolymers, such as, for example, block, graft, random and alternating copolymers; and terpolymers; and blends and modifications thereof. Furthermore, unless otherwise specifically limited, the term “polymer” shall include all possible geometrical configurations of the material. These configurations include, but are not limited to isotactic, syndiotactic, and random symmetries.

“Polynucleotide” refers to a polymer composed of nucleotide units. Polynucleotides include naturally-occurring nucleic acids, such as deoxyribonucleic acid (“DNA”) and ribonucleic acid (“RNA”) as well as nucleic acid analogs. Nucleic acid analogs include those which include non-naturally-occurring bases, nucleotides that engage in linkages with other nucleotides other than the naturally-occurring phosphodiester bond or which include bases attached through linkages other than phosphodiester bonds. Thus, nucleotide analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like. Such polynucleotides can be synthesized, for example, using an automated DNA synthesizer. The term “nucleic acid” typically refers to large polynucleotides. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′ end of the coding RNA transcript are referred to as “downstream sequences.”

“Complementary” refers to the topological compatibility or matching together of interacting surfaces of two polynucleotides. Thus, the two molecules can be described as complementary, and furthermore, the contact surface characteristics are complementary to each other. A first polynucleotide is complementary to a second polynucleotide if the nucleotide sequence of the first polynucleotide is substantially identical to the nucleotide sequence of the polynucleotide binding partner of the second polynucleotide, or if the first polynucleotide can hybridize to the second polynucleotide under stringent hybridization conditions.

A “vector” is a polynucleotide that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), wherein additional DNA segments can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide.

A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06. A nucleotide sequence is “operably linked” to a regulatory sequence if the regulatory sequence affects the expression (e.g., the level, timing, or location of expression) of the nucleotide sequence.

A “host cell” is a cell that can be used to express a polynucleotide of the disclosure. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Typically, a host cell is a cultured cell that can be transformed or transfected with a polypeptide-encoding nucleic acid, which can then be expressed in the host cell. The phrase “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein.

The term “isolated molecule” (where the molecule is, for example, a polypeptide or a polynucleotide) is a molecule that by virtue of its origin or source of derivation (1) is not associated with naturally-associated components that accompany it in its native state, (2) is substantially free of other molecules from the same species, (3) is expressed by a cell from a different species, or (4) does not occur in nature. Thus, a molecule that is chemically synthesized, or expressed in a cellular system different from the cell from which it naturally originates, will be “isolated” from its naturally-associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

A protein or polypeptide is “substantially pure,” “substantially homogeneous,” or “substantially purified” when at least about 60% to 75% of a sample exhibits a single species of polypeptide. The polypeptide or protein may be monomeric or multimeric. A substantially pure polypeptide or protein will typically comprise about 50%, 60%, 70%, 80% or 90% W/W of a protein sample, more usually about 95%, and preferably will be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

The terms “label” or “labeled” as used herein refers to the incorporation of another molecule in the antibody. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In another embodiment, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents such as gadolinium chelates, toxins such as pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In various embodiments, labels are attached by spacer arms of various lengths to reduce potential steric hindrance.

The term “heterologous” as used herein refers to a composition or state that is not native or naturally found, for example, that may be achieved by replacing an existing natural composition or state with one that is derived from another source. Similarly, the expression of a protein in an organism other than the organism in which that protein is naturally expressed constitutes a heterologous expression system and a heterologous protein.

It is understood that aspect and embodiments of the disclosure described herein include “consisting” and/or “consisting essentially of” aspects and embodiments.

Reference to “about” a value or parameter herein includes (and describes) variations that are directed to that value or parameter per se. For example, description referring to “about X” includes description of “X”.

As used herein and in the appended claims, the singular forms “a,” “or,” and “the” include plural referents unless the context clearly dictates otherwise. It is understood that aspects and variations of the disclosure described herein include “consisting” and/or “consisting essentially of” aspects and variations.

### PD1 Blocking Antibody

In one aspect, the PD1 blocking antibody guides the IL-2 moiety of the VitoKine to the TILs in the tumor microenvironment (TME) and restrict the activation of the VitoKine locally to improve the therapeutic index. In another aspect, the PD1 blocking antibody guides the IL-2 moiety of the immunocytokine to the TILs in the TME. In various embodiments, the PD1 blocking antibodies were optimized through modifications in the variable domains of pembrolizumab. In various embodiments, the modifications involved germline sequence substitutions of the CDR residues, germline sequence substitutions of the framework residues, and adoption of the most prevalent and better behaving VH3 human germline family sequence as the acceptor framework. In various embodiments, these modifications were implemented individually or in combination to develop optimized PD1 blocking antibodies. In various embodiments, these optimized PD1 blocking antibodies exhibit a high binding affinity to PD1, function to inhibit PD1 with equal or comparable potency as pembrolizumab, have a higher sequence similarity score to its closest human germline sequence, resulting in an improved degree of humanness compared to pembrolizumab, and are predicted to have lower hydrophobicity, leading to a reduced aggregation propensity than pembrolizumab. In various embodiments, PD1 Ab-IL-2 VitoKine constructs and PD1-targeted IL-2 immunocytokines based on these optimized PD1 blocking antibodies are predicted to have enhanced developability properties. In various embodiments, the PD1 antibody comprises a light chain variable region with the sequence selected from the group of sequences set forth in SEQ ID NOS: 3-5, and a heavy chain variable region with the sequence selected from the group of sequences set forth in SEQ ID NOS: 7-18. In various embodiments, the PD1 antibody comprises a light chain sequence set forth in SEQ ID NO: 44, and a heavy chain with the sequence selected from the group of sequences set forth in SEQ ID NOS: 45-49.

### IL-2 Domain

Interleukin-2 (IL-2), a classic Th1 cytokine, is produced by T cells after activation through the T-cell antigen receptor and the co-stimulatory molecule CD28. The regulation of IL-2 occurs through activation of signaling pathways and transcription factors that act on the IL-2 promoter to generate new gene transcription, but also involves modulation of the stability of IL-2 mRNA. IL-2 binds to a multichain receptor, including a highly regulated α chain and β and γ chains that mediate signaling through the Jak-STAT pathway. IL-2 delivers activation, growth, and differentiation signals to T cells, B cells, and NK cells. IL-2 is also important in mediating activation-induced cell death of T cells, a function that provides an essential mechanism for terminating immune responses. A commercially available unglycosylated human recombinant IL-2 product, aldesleukin (available as the PROLEUKIN® brand of des-alanyl-1, serine-125 human interleukin-2 from Prometheus Laboratories Inc., San Diego Calif.), has been approved for administration to patients suffering from metastatic renal cell carcinoma and metastatic melanoma. IL-2 has also been suggested for administration in patients suffering from or infected with hepatitis C virus (HCV), human immunodeficiency virus (HIV), acute myeloid leukemia, non-Hodgkin's lymphoma, cutaneous T-cell lymphoma, juvenile rheumatoid arthritis, atopic dermatitis, breast cancer, and bladder cancer. Unfortunately, short half-life and severe toxicity limits the optimal dosing of IL-2.

As used herein, the terms “native IL-2” and “native interleukin-2” in the context of proteins or polypeptides refer to any naturally-occurring mammalian interleukin-2 amino acid sequences, including immature or precursor and mature forms. Non-limiting examples of GenBank Accession Nos. for the amino acid sequence of various species of native mammalian interleukin-2 include NP_032392.1 (Mus musculus, immature form), NP_001040595.1 (Macaca mulatta, immature form), NP_000577.2 (human, precursor form), CAA01199.1 (human, immature form), and AAD48509.1 (human, immature form). In various embodiments of the present invention, native IL-2 is the immature or precursor form of a naturally-occurring mammalian IL-2. In other embodiments, native IL-2 is the mature form of a naturally-occurring mammalian IL-2. In various embodiments, native IL-2 is the precursor form of naturally-occurring human IL-2. In various embodiments, native IL-2 is the mature form of naturally-occurring human IL-2. In various embodiments, the IL-2 in the VitoKine and immunocytokine constructs of the present invention is derived from the amino acid sequence of the human IL-2 mature sequence set forth in SEQ ID NO: 116:

(SEQ ID NO: 116)
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT

In various embodiments, the IL-2 domain will be an IL-2 variant (or mutant) comprising a sequence derived from the sequence of the mature human IL-2 polypeptide as set forth in SEQ ID NO: 116. In various embodiments, the IL-2 variant comprises a single C125I amino acid substitution that universally enhances the developability of the protein while fully preserving its biological activity. In various embodiments, the IL-2 variant comprising a single C125I mutation has the amino acid sequence set forth in SEQ ID NO: 117.

In various embodiments, the sequence of the IL-2 variant has at least one amino acid change, e.g., substitution or deletion, compared to the native IL-2 sequence, such changes resulting in IL-2 agonist or antagonist activity. IL-2 agonists are exemplified by comparable or increased biological activity compared to wild type IL-2. IL-2 antagonists are exemplified by decreased biological activity compared to wild type IL-2 or by the ability to inhibit IL-2-mediated responses. In various embodiments, the IL-2 variant has the amino acid sequence derived from SEQ ID NO: 117 with altered binding to IL-2Rα. In various embodiments, the IL-2 variant with altered binding to IL-2Rα comprises the amino acid sequence set forth in SEQ ID NOS: 118-125. In various embodiments, the IL-2 variant has the amino acid sequence derived from SEQ ID NO: 117 with reduced/abolished binding to IL-2Rα to selectively activate and proliferate effector T cells (Teff) for treating cancer. In various embodiments, the IL-2 variant with reduced/abolished binding to IL-2Rα comprises the amino acid sequence set forth in SEQ ID NOS: 118-122. In various embodiments, the IL-2 variant has the amino acid sequence derived from SEQ ID NO: 117 with reduced binding to IL-2Rβ and/or γc. In various embodiments, the IL-2 variant reduced binding to IL-2Rβ and/or γ comprises the amino acid sequence set forth in SEQ ID NOS: 126-150. In various embodiments, the IL-2 variant has the amino acid sequence derived from SEQ ID NO: 117 with reduced/abolished binding to IL-2Rα and reduced binding to IL-2Rβ and/or γc. In various embodiments, the IL-2 variant with reduced/abolished binding to IL-2Rα and reduced binding to IL-2Rβ and/or γc comprises the amino acid sequence set forth in SEQ ID NOS: 151-180. As will be appreciated by those in the art, all of the mutations can be optionally and independently combined in any way to achieve optimal affinity and activity modulation.

### IL-2Rα Domain (Concealing Moiety Domain in PD1 Ab-IL-2 VitoKine)

The IL-2 receptor (IL-2R) is a heterotrimeric protein expressed on the surface of certain immune cells, such as lymphocytes, that binds and responds to IL-2. IL-2R has three subunits: α (CD25), β (CD122), and γ (CD132 or the common gamma chain (γc)), a shared chain with five other cytokine receptors: IL-4R, IL-7R, IL-9R, IL-15R, and IL-21R). Alpha chain (alias: Tac antigen or p55) of human receptor is encoded on chromosome 10p14-15 by the gene IL-2RA. The gene for the human p chain (IL-2RB, CD122) of the receptor is located on chromosome 22q11.2-12, while the gene for the human γ chain (IL-2RG) is on chromosome Xq13. Assembly of all three subunits of the receptor is important for the signal transduction into the B and T cells. IL-2R was found on the cell surface (either temporary or permanent) in almost all hematopoietic cells including lymphoid linages T, B, and NK cells, as well as myeloid ones like macrophages, monocytes, and neutrophils. The signal is transferred into the cell via the Janus kinases—Jak1 and Jak3. The phosphorylation of the intracytosolic part of the receptor's β chain enables homodimer formation of STAT-3 and STAT-5 factors. Homodimers of STAT-3 and STAT-5 show increased affinity for the nucleus, where they bind to specific DNA elements enhancing the transcription of IL-2-dependent genes.

As used herein, the terms “native IL-2Rα” and “native interleukin-2 receptor alpha” in the context of proteins or polypeptides refer to any naturally-occurring mammalian interleukin-2 receptor alpha (“IL-2Rα”) amino acid sequence, including immature or precursor and mature forms and naturally-occurring isoforms. Non-limiting examples of GenBank Accession Nos. for the amino acid sequence of various native mammalian IL-2Rα include NP_032393.3 (Mus musculus), CAK26553.1 (human), and NP_000408.1 (human). In various embodiments, the IL-2Rα domain is derived from the amino acid sequence of the human IL-2Rα sequence set forth in SEQ ID NO: 181:

(SEQ ID NO: 181)
MDSYLLMWGLLTFIMVPGCQAELCDDDPPEIPHATFKAMAYKEGTMLNCE
CKRGFRRIKSGSLYMLCTGNSSHSSWDNQCQCTSSATRNTTKQVTPQPEE
QKERKTTEMQSPMQPVDQASLPGHCREPPPWENEATERIYHFVVGQMVYY
QCVQGYRALHRGPAESVCKMTHGKTRWTQPQLICTGEMETSQFPGEEKPQ
ASPEGRPESETSCLVTTTDFQIQTEMAATMETSIFTTEYQVAVAGCVFLL
ISVLLLSGLTWQRRQRKSRRTI

In various embodiments, a concealing moiety domain (D3) is used to reversibly conceal the activity of the IL-2 domain in the PD1 Ab-IL-2 VitoKine construct. In various embodiments, the concealing moiety domain is an IL-2Rα extracellular domain or a functional fragment thereof. In various preferred embodiments, the concealing moiety domain is an IL-2RαSushi domain comprising the amino acid sequence of the mature human IL-2Rα polypeptide as set forth in SEQ ID NO: 182. In various preferred embodiments, the concealing moiety domain is a variant of IL-2RαSushi domain.

(SEQ ID NO: 182)
ELCDDDPPEIPHATFKAMAYKEGTMLNCECKRGFRRIKSGSLYMLCTGNS
SHSSWDNQCQCTSSATRNTTKQVTPQPEEQKERKTTEMQSPMQPVDQASL
PGHCREPPPWENEATERIYHFVVGQMVYYQCVQGYRALHRGPAESVCKMT
HGKTRWTQPQLICTG

In various embodiments, PD1 Ab-IL-2 VitoKine comprises IL-2RαSushi (SEQ ID NO: 182) as the concealing moiety domain to conceal IL-2 (including IL-2 variants) activity. Although wild-type IL-2Rα binds to IL-2 with a moderate affinity of 30 nM, there remains a possibility that, upon cleaving the linker, IL-2Rα may not dissociate. The association between the cleaved IL-2Rα and IL-2 could reduce the activity of IL-2 and/or tilt the balance of T cell subpopulations toward an undesired outcome. With affinity reducing mutation(s) introduced into IL-2RαSushi, the IL-2Rα sushi domains are likely to dissociate away from the IL-2 upon linker cleavage. In various embodiments, the concealing moiety domain in PD1 Ab-IL-2 VitoKine is IL-2RαSushi variant comprising IL-2-binding-weakening mutations, e.g., R36A, K38E, L42G, or Y43A, or any combination of the substitutions. In various embodiments, the IL-2RαSushi variant can effectively conceal IL-2 moiety domain's activity despite its reduced affinity to IL-2. In various embodiments, the IL-2RαSushi variant is anticipated to dissociate and diffuse away from IL-2 upon linker cleavage because of its reduced affinity to IL-2.

In various embodiments, the PD1 Ab-IL-2 VitoKine constructs of the present invention contain a concealing moiety domain that is one of the IL-2RαSushi domain variants comprising the amino acid sequence as set forth in SEQ ID NOS: 183-185.

### L1 and L2 Linkers in PD1 Ab-IL-2 VitoKine

### Cleavable Linkers

A cleavable linker, or a linker susceptible to a disease-associated enzyme, may contain a moiety, e.g., a protein substrate, capable of being specifically cleaved by a protease that is present at elevated levels at the disease site as compared to non-disease tissues. Literature contains multiple reports on increased levels of enzymes with known substrates in various types of cancers, e.g., solid tumors. See, e.g., La Rocca et al., Brit. J. Cancer 90:1414-1421 and Ducry et al., Bioconjug. Chem. 21:5-13, 2010, each of which is incorporated by reference herein in its entirety. In various embodiments, the protease capable of cleaving a protease-cleavable linker is selected from the group consisting of metalloproteinase, e.g., matrix metalloproteinase (MMP) 1-28, serine protease, e.g., urokinase-type plasminogen activator (uPA) and matriptase, cysteine protease, e.g., legumain, aspartic protease, and cathepsin protease. Exemplary proteases are provided in Table 2:

TABLE 2

| Protease family | Protease | RefSeq (protein) |
|---|---|---|
| Matrix | MMP-1 (Collagenase 1) | NP_002412 |
| Metalloproteins | MMP-2 (Gelatinase A) | NP_001121363 |
| (MMPs) | MMP-3 (Stromelysin 1) | NP_002413 |
| | MMP-7 (Matrilysin 1) | NP_002414 |
| | MMP-8 (Collagenase 2) | NP_002415 |
| | MMP-9 (Gelatinase B) | NP_004985 |
| | MMP-10 (Stromelysin 2) | NP_002416 |
| | MMP-11 (Stromelysin 3) | NP_005931.2 |
| | MMP-12 (Macrophage | NP_002417.2 |
| | Elastase) | |
| | MMP-13 (Collagenase 3) | NP_002418 |
| | MMP-14 (MT1-MMP) | NP_004986 |
| | MMP-15 (MT2-MMP) | NP_002419 |
| | MMP-19 | NP_002420 |
| | MMP-23 (CA-MMP) | NP_008914 |
| | MMP-24 (MT5-MMP) | NP_006681 |
| | MMP-26 (Matrilysin 2) | NP_068573.2 |
| | MMP-27 (CMMP) | NP_071405.2 |
| Cysteine | Legumain | NP_001008530 |
| Proteases | Cathepsin C | NP_001805.3 |
| | Cathepsin K | NP_000387 |
| | Cathepsin L1 | NP_001903 |
| | Cathepsin S | NP_004070 |
| | Cathepsin X (Cathepsin Z) | NP_001327.2 |
| Aspartase | Cathepsin D | NP_001900 |
| Proteases | Cathepsin E | NP_001901 |
| | Secretase (BACE1) | NP_001193978 |
| Serine | Urokinase plasminogen | NM_002658 |
| Proteases | activator (uPA) | |
| | Tissue-type plasminogen | NP_000921 |
| | activator (tPA) | |
| | Plasmin | NP_000292 |
| | Thrombin | NP_000497 |
| | Prostate-specific antigen | NP_001639 |
| | (PSA, KLK3) | |
| | human neutrophil elastase | NP_001963 |
| | (HNE) | |
| | Elastase (CELA1) | NP_001962.3 |
| | Tryptase | NP_003285.2 |
| | Matriptase (ST14) | NP_068813 |
| Disintegrin and | ADAM-10 | NP_001101 |
| metalloproteinase | ADAM-17 | NP_003174 |
| (ADAM) | | |

Exemplary protease substrate peptide sequences, which can be used as protease cleavable linkers with or without peptide spacers, are provided in Table 3:

TABLE 3

| Proteases | Substrate peptide | SEQ ID NO: |
|---|---|---|
| MMP-2, 7, 9, 14 | SPLGLAGS | 54 |
| MMP-2, 7, 9, 14, | EPLELRAG | 55 |
| matriptase | | |
| Matriptase, uPA, | LSGRSDNH | 56 |
| Legumain | | |
| MMP-2 | GPLGIAGQ | 57 |
| MMP-2, 14 | GTAHLMGG | 58 |
| MMP-14 | RIGSLRTA | 59 |
| MMP-14 | SGRSENIRTA | 60 |
| MMP-2, 9 | GPLGMLSQ | 61 |
| MMP-2, 9 | GPAGMKGL | 62 |
| MMP-9, uPA | RPSASRSA | 63 |
| MMP | PLGLAG | 64 |
| uPA | LGGSGRSANAILE | 65 |
| uPA | GGSGRSANAI | 66 |
| uPA | SGRSA | 67 |
| Legumain | AANL | 68 |
| Legumain | GPTNKVR | 69 |
| Cathepsin C | GFFY | 70 |
| Cathepsin D | GPICFRLG | 71 |
| Cathepsin E | RQAGFSL | 72 |
| Matriptase | RQARAVGG | 73 |
| Matriptase | PMAKK | 74 |
| Prostate Specific | HSSKLQ | 75 |
| antigen | | |
| MMP-2, 9 and | GPLGMLSQPMAKK | 76 |
| Matriptase | | |
| Matriptase and | PMAKKGPLGMLSQ | 77 |
| MMP-2,9 | | |

In various embodiments, the protease is MMP-9 or MMP-2. In a further specific embodiment, the protease is matriptase. In a further specific embodiment, the protease is MMP-14. In further specific embodiment, the protease is legumain. In various embodiments, the protease cleavable linker may contain two or more protease substrate sequences. In various embodiments, the proteases are MMP-2/MMP-9 and matriptase. In various embodiments, the protease-cleavable linker comprises the protease recognition sequence ‘GPLGMLSQ’ (SEQ ID NO: 61). In various embodiments, the protease-cleavable linker comprises the protease recognition sequence ‘SGRSENIRTA’ (SEQ ID NO: 60). In various embodiments, the protease-cleavable linker comprises the protease recognition sequence ‘GPTNKVR’ (SEQ ID NO: 69). In various embodiments, the protease-cleavable linker comprises the protease recognition sequence ‘PMAKK’ (SEQ ID NO: 74). In various embodiments, the protease-cleavable linker comprises the protease recognition sequence ‘GPLGMLSQPMAKK’ (SEQ ID NO: 76). In various embodiments, the protease-cleavable linker comprises the protease recognition sequence ‘PMAKKGPLGMLSQ’ (SEQ ID NO: 77).

In various embodiments, peptide spacers may be incorporated on either side of a protease cleavable sequence or to flank both sides of a protease cleavable sequence, or as a non-cleavable linker without a protease substrate site. Peptide spacer serves to position a cleavable linker, making it more readily accessible to the enzyme responsible for cleavage. The length and composition of a peptide spacer can be fine-tuned to balance the accessibility for enzymatic cleavage and the spatial constraint required to reversibly conceal the D2 domain from exerting its biological activity. A peptide spacer may include 1-100 amino acids. Suitable peptide spacers are known in the art, which include, but are not limited to, peptide linkers containing flexible amino acid residues, such as glycine and serine. In various embodiments, a peptide spacer can contain 1 to 12 amino acids including motifs of G, S, GSGG (SEQ ID NO: 104), GGSS (SEQ ID NO: 105), GSGS (SEQ ID NO: 109), GSGSGS (SEQ ID NO: 110), GSGSGSGS (SEQ ID NO: 111), GSGSGSGSGS (SEQ ID NO: 112), or GSGSGSGSGSGS (SEQ ID NO: 113). In other embodiments, a peptide spacer can contain motifs of (GGGGS)(SEQ ID NO: 106)ₙ, wherein n is an integer from 1 to 10. In other embodiments, a peptide spacer can also contain amino acids other than glycine and serine. A peptide spacer is stable under physiological conditions as well as at a diseased site, such as a cancer site.

Exemplary protease cleavable linkers with peptide spacers flanking protease substrate peptides (underscored) are provided in Table 4:

TABLE 4

| Protease cleavable linker | SEQ ID NO: |
|---|---|
| GGGSGGGGSGGGGSLSGRSDNHGGSGGGGS | 78 |
| GSSSGRSENIRTAGT | 79 |
| GGGGSGGGGSGGGSLGGSGRSANAILEGGSGGGGS | 80 |
| GGGGSGGGGSLGGSGRSANAILEGGGGS | 81 |
| GGGGSLGGSGRSANAILEGGS | 82 |
| GGGSGPTNKVRGGS | 83 |
| GGSGPLGMLSQGGGS | 84 |
| GGPLGMLSQS | 85 |
| GGGPLGMLSQGGS | 86 |
| GGPTNKVRGS | 87 |
| GRQARAVGGS | 88 |
| GGGSGRSENIRTAGG | 89 |
| SGGPGPAGMKGLPGS | 90 |
| GGGGSPMAKKGGGGS | 91 |
| GGPLGMLSQPMAKKS | 92 |
| GGSGPLGMLSQPMAKKGGGS | 93 |
| GGGPMAKKGPLGMLSQGGGS | 94 |

### Non-Cleavable Linkers

Non-cleavable linker provides covalent linkage and additional structural and/or spatial flexibility between protein domains. As is known in the art, peptide linkers containing flexible amino acid residues, such as glycine and serine, can be used as non-cleavable linkers. In various embodiments, non-cleavable linker may include 1-100 amino acids. In various embodiments, a spacer can contain motifs of GSGG (SEQ ID NO: 104), GGSS (SEQ ID NO: 105), GSGS (SEQ ID NO: 109), GSGSGS (SEQ ID NO: 110), GSGSGSGS (SEQ ID NO: 111), GSGSGSGSGS (SEQ ID NO: 112), or GSGSGSGSGSGS (SEQ ID NO: 113). In other embodiments, a spacer can contain motifs of (GGGGS)(SEQ ID NO: 106)ₙ, wherein n is an integer from 1 to 10. In other embodiments, a linker can also contain amino acids other than glycine and serine. In another embodiment, the non-cleavable linker can be a simple chemical bond, e.g., an amide bond (e.g., by chemical conjugation of PEG). A non-cleavable linker is stable under physiological conditions as well as at a diseased site, such as a cancer site.

Exemplary non-cleavable linkers are provided in Table 5:

TABLE 5

| | SEQ ID |
|---|---|
| Linker sequence | NO: |
| EPKSSDKTHTSPPS | 95 |
| GGGSGGGSGGGS | 96 |
| GGGS | 97 |
| GSSGGSGGS | 98 |
| GSSGT | 99 |
| GGGGSGGGGSGGGS | 100 |
| AEAAAKEAAAKEAAAKA | 101 |
| GGGGSGGGGSGGGGSGGGGS | 102 |
| GGGSGGGS | 103 |
| GSGG | 104 |
| GGSS | 105 |
| GGGGS | 106 |
| GGSGG | 107 |
| SGGG | 108 |
| GSGS | 109 |
| GSGSGS | 110 |
| GSGSGSGS | 111 |
| GSGSGSGSGS | 112 |
| GSGSGSGSGSGS | 113 |
| GGGGSGGGGS | 114 |
| GGGGSGGGGSGGGGS | 115 |

A combination of cleavable and non-cleavable Linkers

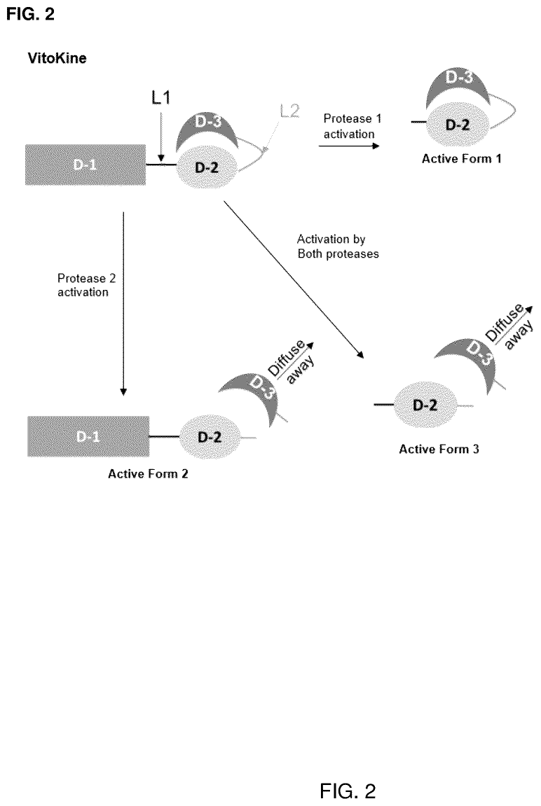

In various embodiments, the L1 and L2 linkers can be both cleavable or a combination of cleavable and non-cleavable linkers to yield different forms of active moiety of the IL-2 domain to fulfill specific therapeutic objectives, optimize the risk to benefit ratio, or align with diverse properties of the cytokine. The exemplary active forms released by cleavage of the linkers are depicted in FIG. 2. The active form 1 derived from cleavage of the L1 linker and the active form 3 derived from cleavage of L1 and L2 linkers are both short-acting cytokines due to their release from the targeting antibody after proteolysis. The presence or absence of the concealing domain would result in distinct activity for these two active forms in the local environment. After acting locally, the short-acting active forms can be eliminated from systemic circulation quickly, leading to reduced toxicities. In contrast, Active Form 2 derived from the cleavage of the L2 linker (depicted in FIG. 2) is a functionally fully restored IL-2 fused to the PD1 Ab at or near the disease site. This active form is capable of cis-activating IL-2R signaling on PD1-expressing T cell at or near the disease site, which synergistically enhances the two pathways and boosts the anticancer immune response, while minimizing systematic toxicity.

### Polynucleotides

In another aspect, the present disclosure provides isolated nucleic acid molecules comprising a polynucleotide of IL-2, an IL-2 variant, IL-2Rα, an IL-2Rα variant, an PD1 blocking antibody, an antibody fragment, a PD1 Ab-IL-2 VitoKine construct, or a PD1-targeted IL-2 immunocytokine of the present disclosure. The subsequent paragraphs of this sub-section “polynucleotides” will utilize PD1-targeted IL-2 VitoKine (VitoKine) constructs as illustrative instances, yet these concepts shall equally be applicable to the PD1-targeted IL-2 immunocytokines of the current invention.

The subject nucleic acids may be single-stranded or double stranded. Such nucleic acids may be DNA or RNA molecules. DNA includes, for example, cDNA, genomic DNA, synthetic DNA, DNA amplified by PCR, and combinations thereof. Genomic DNA encoding VitoKine constructs is obtained from genomic libraries which are available for a number of species. Synthetic DNA is available from chemical synthesis of overlapping oligonucleotide fragments followed by assembly of the fragments to reconstitute part or all of the coding regions and flanking sequences. RNA may be obtained from prokaryotic expression vectors which direct high-level synthesis of mRNA, such as vectors using T7 promoters and RNA polymerase. The DNA molecules of the disclosure include full-length genes as well as polynucleotides and fragments thereof. The full-length gene may also include sequences encoding the N-terminal signal sequence. Such nucleic acids may be used, for example, in methods for making the novel VitoKine constructs.

In various embodiments, the isolated nucleic acid molecules comprise the polynucleotides described herein, and further comprise a polynucleotide encoding at least one heterologous protein described herein. In various embodiments, the nucleic acid molecules further comprise polynucleotides encoding the linkers or hinge linkers described herein.

In various embodiments, the recombinant nucleic acids of the present disclosure may be operably linked to one or more regulatory nucleotide sequences in an expression construct. Regulatory sequences are art-recognized and are selected to direct expression of the VitoKine construct. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the present disclosure. The promoters may be either naturally-occurring promoters, or hybrid promoters that combine elements of more than one promoter. An expression construct may be present in a cell on an episome, such as a plasmid, or the expression construct may be inserted in a chromosome. In various embodiments, the expression vector contains a selectable marker gene to allow the selection of transformed host cells. Selectable marker genes are well known in the art and will vary with the host cell used.

In another aspect of the present disclosure, the subject nucleic acid is provided in an expression vector comprising a nucleotide sequence encoding a VitoKine construct and operably linked to at least one regulatory sequence. The term “expression vector” refers to a plasmid, phage, virus or vector for expressing a polypeptide from a polynucleotide sequence. Vectors suitable for expression in host cells are readily available and the nucleic acid molecules are inserted into the vectors using standard recombinant DNA techniques. Such vectors can include a wide variety of expression control sequences that control the expression of a DNA sequence when operatively linked to it may be used in these vectors to express DNA sequences encoding a VitoKine construct. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., PhoS, the promoters of the yeast a-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered. An exemplary expression vector suitable for expression of VitoKine is the pDSRa, and its derivatives, containing VitoKine polynucleotides, as well as any additional suitable vectors known in the art or described below.

A recombinant nucleic acid of the present disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant VitoKine construct include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the B-gal containing pBlueBac Ill).

In various embodiments, a vector will be designed for production of the subject VitoKine construct in Chinese Hamster Ovary (CHO) cells or Human Embryonic Kidney 293 (HEK293) cells, such as a Pcmv-Script vector (Stratagene, La Jolla, Calif.), pcDNA4 vectors (Invitrogen, Carlsbad, Calif.) and pCl-neo vectors (Promega, Madison, Wis.). As will be apparent, the subject gene constructs can be used to cause expression of the subject VitoKine constructs in cells propagated in culture, e.g., to produce proteins, including fusion proteins or variant proteins, for purification.

This present disclosure also pertains to a host cell transfected with a recombinant gene including a nucleotide sequence coding an amino acid sequence for one or more of the subject VitoKine construct. The host cell may be any prokaryotic or eukaryotic cell. For example, a VitoKine construct of the present disclosure may be expressed in bacterial cells such as E. coli, insect cells (e.g., using a baculovirus expression system), yeast, or mammalian cells. Other suitable host cells are known to those skilled in the art, such as CHO cells, or HEK293 cells.

Accordingly, the present disclosure further pertains to methods of producing the subject VitoKine constructs. For example, a host cell transfected with an expression vector encoding a VitoKine construct can be cultured under appropriate conditions to allow expression of the VitoKine construct to occur. The VitoKine construct may be secreted and isolated from a mixture of cells and medium containing the VitoKine construct. Alternatively, the VitoKine construct may be retained cytoplasmically or in a membrane fraction and the cells harvested, lysed and the protein isolated. A cell culture includes host cells, media and other byproducts. Suitable medias for cell culture are well known in the art.

The polypeptides and proteins of the present disclosure can be purified according to protein purification techniques are well known to those of skill in the art. These techniques involve, at one level, the crude fractionation of the proteinaceous and non-proteinaceous fractions. Having separated the peptide polypeptides from other proteins, the peptide or polypeptide of interest can be further purified using chromatographic and electrophoretic techniques to achieve partial or complete purification (or purification to homogeneity). The term “isolated polypeptide” or “purified polypeptide” as used herein, is intended to refer to a composition, isolatable from other components, wherein the polypeptide is purified to any degree relative to its naturally-obtainable state. A purified polypeptide therefore also refers to a polypeptide that is free from the environment in which it may naturally occur. Generally, “purified” will refer to a polypeptide composition that has been subjected to fractionation to remove various other components, and which composition substantially retains its expressed biological activity. Where the term “substantially purified” is used, this designation will refer to a peptide or polypeptide composition in which the polypeptide or peptide forms the major component of the composition, such as constituting about 50%, about 60%, about 70%, about 80%, about 85%, or about 90% or more of the proteins in the composition.

Various techniques suitable for use in purification will be well known to those of skill in the art. These include, for example, precipitation with ammonium sulphate, PEG, antibodies (immunoprecipitation) and the like or by heat denaturation, followed by centrifugation; chromatography such as affinity chromatography (Protein-A columns), ion exchange, gel filtration, reverse phase, hydroxylapatite, hydrophobic interaction chromatography; isoelectric focusing; gel electrophoresis; and combinations of these techniques. As is generally known in the art, it is believed that the order of conducting the various purification steps may be changed, or that certain steps may be omitted, and still result in a suitable method for the preparation of a substantially purified polypeptide.

### Pharmaceutical Compositions

The subsequent paragraphs of this sub-section “Pharmaceutical Compositions” will utilize PD1-targeted IL-2 VitoKine (VitoKine) constructs as illustrative instances, yet these concepts shall equally be applicable to the PD1-targeted IL-2 immunocytokines of the current invention.

In another aspect, the present disclosure provides a pharmaceutical composition comprising the VitoKine constructs in admixture with a pharmaceutically acceptable carrier. Such pharmaceutically acceptable carriers are well known and understood by those of ordinary skill and have been extensively described (see, e.g., Remington's Pharmaceutical Sciences, 18th Edition, A. R. Gennaro, ed., Mack Publishing Company, 1990). The pharmaceutically acceptable carriers may be included for purposes of modifying, maintaining, or preserving, for example, the pH, osmolarity, viscosity, clarity, color, isotonicity, odor, sterility, stability, rate of dissolution or release, adsorption, or penetration of the composition. Such pharmaceutical compositions may influence the physical state, stability, rate of in vivo release, and rate of in vivo clearance of the polypeptide. Suitable pharmaceutically acceptable carriers include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin, or immunoglobulins); coloring; flavoring and diluting agents; emulsifying agents; hydrophilic polymers (such as polyvinylpyrrolidone); low molecular weight polypeptides; salt-forming counter ions (such as sodium); preservatives (such as benzalkonium chloride, benzoic acid, salicylic acid, thimerosal, phenethyl alcohol, methylparaben, propylparaben, chlorhexidine, sorbic acid, or hydrogen peroxide); solvents (such as glycerin, propylene glycol, or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants.

The primary vehicle or carrier in a pharmaceutical composition may be either aqueous or non-aqueous in nature. For example, a suitable vehicle or carrier may be water for injection, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute thereof. In one embodiment of the present disclosure, compositions may be prepared for storage by mixing the selected composition having the desired degree of purity with optional formulation agents (Remington's Pharmaceutical Sciences, supra) in the form of a lyophilized cake or an aqueous solution. Further, the therapeutic composition may be formulated as a lyophilizate using appropriate excipients such as sucrose. The optimal pharmaceutical composition will be determined by one of ordinary skill in the art depending upon, for example, the intended route of administration, delivery format, and desired dosage.

When parenteral administration is contemplated, the therapeutic pharmaceutical compositions may be in the form of a pyrogen-free, parenterally acceptable aqueous solution comprising the desired VitoKine construct in a pharmaceutically acceptable vehicle. A particularly suitable vehicle for parenteral injection is sterile distilled water in which a polypeptide is formulated as a sterile, isotonic solution, properly preserved. In various embodiments, pharmaceutical formulations suitable for injectable administration may be formulated in aqueous solutions, preferably in physiologically compatible buffers such as Hanks' solution, Ringer's solution, or physiologically buffered saline. Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Optionally, the suspension may also contain suitable stabilizers or agents to increase the solubility of the compounds and allow for the preparation of highly concentrated solutions.

In various embodiments, the therapeutic pharmaceutical compositions may be formulated for targeted delivery using a colloidal dispersion system. Colloidal dispersion systems include macromolecule complexes, nanocapsules, microspheres, beads, and lipid-based systems including oil-in-water emulsions, micelles, mixed micelles, and liposomes. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

In various embodiments, oral administration of the pharmaceutical compositions is contemplated. Pharmaceutical compositions that are administered in this fashion can be formulated with or without those carriers customarily used in the compounding of solid dosage forms such as tablets and capsules. In solid dosage forms for oral administration (capsules, tablets, pills, dragees, powders, granules, and the like), one or more therapeutic compounds of the present disclosure may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

In various embodiments, topical administration of the pharmaceutical compositions, either to skin or to mucosal membranes, is contemplated. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface-active agents. Keratolytic agents such as those known in the art may also be included. Examples are salicylic acid and sulfur. Dosage forms for the topical or transdermal administration include powders, sprays, ointments, pastes, creams, lotions, gels, solutions, patches, and inhalants. The active compound may be mixed under sterile conditions with a pharmaceutically acceptable carrier, and with any preservatives, buffers, or propellants which may be required. The ointments, pastes, creams and gels may contain, in addition to a subject compound of the disclosure (e.g., a VitoKine construct), excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Additional pharmaceutical compositions contemplated for use herein include formulations involving polypeptides in sustained- or controlled-delivery formulations. In various embodiments, pharmaceutical compositions may be formulated in nanoparticles, as slow-release hydrogel, or incorporated into oncolytic viruses. Such nanoparticles methods include, e.g., encapsulation in nanoparticles composed of polymers with a hydrophobic backbone and hydrophilic branches as drug carriers, encapsulation in microparticles, insertion into liposomes in emulsions, and conjugation to other molecules. Examples of nanoparticles include mucoadhesive nanoparticles coated with chitosan and Carbopol (Takeuchi et al., Adv. Drug Deliv. Rev. 47(1):39-54, 2001) and nanoparticles containing charged combination polyesters, poly(2-sulfobutyl-vinyl alcohol) and poly(D,L-lactic-co-glycolic acid) (Jung et al., Eur. J. Pharm. Biopharm. 50(1):147-160, 2000). Albumin-based nanoparticle compositions have been developed as a drug delivery system for delivering hydrophobic drugs such as a taxane. See, for example, U.S. Pat. Nos. 5,916,596; 6,506,405; 6,749,868; 6,537,579; 7,820,788; and 7,923,536. Abraxane®, an albumin stabilized nanoparticle formulation of paclitaxel, was approved in the United States in 2005 and subsequently in various other countries for treating metastatic breast cancer.

Techniques for formulating a variety of other sustained- or controlled-delivery means, such as liposome carriers, bio-erodible microparticles or porous beads and depot injections, are also known to those skilled in the art.

An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the polypeptide is being used, the route of administration, and the size (body weight, body surface or organ size) and condition (the age and general health) of the patient. Accordingly, the clinician may titer the dosage and modify the route of administration to obtain the optimal therapeutic effect. A typical dosage may range from about 0.0001 mg/kg to up to about 100 mg/kg or more, depending on the factors mentioned above. Polypeptide compositions may be preferably injected or administered intravenously. Long-acting pharmaceutical compositions may be administered every three to four days, every week, biweekly, triweekly, monthly, or even longer durations depending on the half-life and clearance rate of the particular formulation. The frequency of dosing will depend upon the pharmacokinetic parameters of the polypeptide in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

The route of administration of the pharmaceutical composition is in accord with known methods, e.g. orally, through injection by intravenous, intraperitoneal, intratumoral, intracerebral (intra-parenchymal), intracerebroventricular, intramuscular, intra-ocular, intraarterial, intraportal, intralesional routes, intramedullary, intrathecal, intraventricular, intravesical, transdermal, subcutaneous, or intraperitoneal; as well as intranasal, enteral, topical, sublingual, urethral, vaginal, or rectal means, by sustained release systems or by implantation devices. Where desired, the compositions may be administered by bolus injection or continuously by infusion, or by implantation device. Alternatively, or additionally, the composition may be administered locally via implantation of a membrane, sponge, or another appropriate material on to which the desired molecule has been absorbed or encapsulated. Where an implantation device is used, the device may be implanted into any suitable tissue or organ, and delivery of the desired molecule may be via diffusion, timed-release bolus, or continuous administration.

### Therapeutic Uses

The subsequent paragraphs of this sub-section “Therapeutic Uses” will utilize PD1-targeted IL-2 VitoKine (VitoKine) constructs as illustrative instances, yet these concepts shall equally be applicable to the PD1-targeted IL-2 immunocytokines of the current invention.

The present disclosure provides for a method of treating cancer cells in a subject, comprising administering to said subject a therapeutically effective amount (either as monotherapy or in a combination therapy regimen) of a VitoKine construct of the present disclosure in pharmaceutically acceptable carrier, wherein such administration inhibits the growth and/or proliferation of a cancer cell. Specifically, a VitoKine construct of the present disclosure is useful in treating disorders characterized as cancer. Such disorders include, but are not limited to solid tumors, such as cancers of the breast, respiratory tract, brain, reproductive organs, digestive tract, urinary tract, eye, liver, skin, head and neck, thyroid, parathyroid and their distant metastases, lymphomas, sarcomas, multiple myeloma and leukemia. Examples of breast cancer include, but are not limited to invasive ductal carcinoma, invasive lobular carcinoma, ductal carcinoma in situ, and lobular carcinoma in situ. Examples of cancers of the respiratory tract include, but are not limited to, small-cell and non-small-cell lung carcinoma, as well as bronchial adenoma and pleuropulmonary blastoma. Examples of brain cancers include, but are not limited to, brain stem and hypothalamic glioma, cerebellar and cerebral astrocytoma, neuroblastoma, medulloblastoma, ependymoma, as well as neuroectodermal and pineal tumor. Tumors of the male reproductive organs include, but are not limited to, prostate and testicular cancer. Tumors of the female reproductive organs include, but are not limited to endometrial, cervical, ovarian, vaginal, and vulvar cancer, as well as sarcoma of the uterus. Tumors of the digestive tract include, but are not limited to anal, colon, colorectal, esophageal, gallbladder, gastric, liver, breast, pancreatic, rectal, small-intestine, and salivary gland cancers. Tumors of the urinary tract include, but are not limited to bladder, penile, kidney, renal pelvis, ureter, and urethral cancers. Eye cancers include, but are not limited to, intraocular melanoma and retinoblastoma. Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma. Skin cancers include, but are not limited to squamous cell carcinoma, Kaposi's sarcoma, malignant melanoma, Merkel cell skin cancer, and non-melanoma skin cancer. Head-and-neck cancers include, but are not limited to nasopharyngeal cancer, and lip and oral cavity cancer. Lymphomas include, but are not limited to AIDS-related lymphoma, non-Hodgkin's lymphoma, cutaneous T-cell lymphoma, Hodgkin's disease, and lymphoma of the central nervous system. Sarcomas include, but are not limited to, sarcoma of the soft tissue, osteosarcoma, malignant fibrous histiocytoma, lymphosarcoma, and rhabdomyosarcoma. Leukemias include, but are not limited to acute myeloid leukemia, acute lymphoblastic leukemia, chronic lymphocytic leukemia, chronic myelogenous leukemia, and hairy cell leukemia.

In various embodiments, the VitoKine construct can be used as a single agent for treatment of all kinds of cancers, including but not limited to non-small cell lung, small cell lung, melanoma, renal cell carcinoma, urothelial, liver, breast, pancreatic, colorectal, gastric, prostate, and sarcoma.

Therapeutically effective amount” or “therapeutically effective dose” refers to that amount of the therapeutic agent being administered which will relieve to some extent one or more of the symptoms of the disorder being treated.

A therapeutically effective dose can be estimated initially from cell culture assays by determining an IC₅₀ (half maximal inhibitory concentration). A dose can then be formulated in animal models to achieve a circulating plasma concentration range that includes the IC₅₀ as determined in cell culture. Such information can be used to more accurately determine useful doses in humans. Levels in plasma may be measured, for example, by HPLC. The exact composition, route of administration and dosage can be chosen by the individual physician in view of the subject's condition.

Dosage regimens can be adjusted to provide the optimum desired response (e.g., a therapeutic or prophylactic response). For example, a single bolus can be administered, several divided doses (multiple or repeat or maintenance) can be administered over time and the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the present disclosure will be dictated primarily by the unique characteristics of the antibody and the particular therapeutic or prophylactic effect to be achieved.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the present disclosure.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Further, the dosage regimen with the compositions of this disclosure may be based on a variety of factors, including the type of disease, the age, weight, sex, medical condition of the subject, the severity of the condition, and the route of administration. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present disclosure encompasses intra-subject dose-escalation as determined by the skilled artisan. Determining appropriate dosages and regimens are well-known in the relevant art and would be understood to be encompassed by the skilled artisan once provided the teachings disclosed herein.

An exemplary, non-limiting daily dosing range for a therapeutically or prophylactically effective amount of an VitoKine, or VitoKine variant, of the disclosure can be 0.0001 to 100 mg/kg, 0.0001 to 90 mg/kg, 0.0001 to 80 mg/kg, 0.0001 to 70 mg/kg, 0.0001 to 60 mg/kg, 0.0001 to 50 mg/kg, 0.0001 to 40 mg/kg, 0.0001 to 30 mg/kg, 0.0001 to 20 mg/kg, 0.0001 to 10 mg/kg, 0.0001 to 5 mg/kg, 0.0001 to 4 mg/kg, 0.0001 to 3 mg/kg, 0.0001 to 2 mg/kg, 0.0001 to 1 mg/kg, 0.001 to 50 mg/kg, 0.001 to 40 mg/kg, 0.001 to 30 mg/kg, 0.001 to 20 mg/kg, 0.001 to 10 mg/kg, 0.001 to 5 mg/kg, 0.001 to 4 mg/kg, 0.001 to 3 mg/kg, 0.001 to 2 mg/kg, 0.001 to 1 mg/kg, 0.010 to 50 mg/kg, 0.010 to 40 mg/kg, 0.010 to 30 mg/kg, 0.010 to 20 mg/kg, 0.010 to 10 mg/kg, 0.010 to 5 mg/kg, 0.010 to 4 mg/kg, 0.010 to 3 mg/kg, 0.010 to 2 mg/kg, 0.010 to 1 mg/kg, 0.1 to 50 mg/kg, 0.1 to 40 mg/kg, 0.1 to 30 mg/kg, 0.1 to 20 mg/kg, 0.1 to 10 mg/kg, 0.1 to 5 mg/kg, 0.1 to 4 mg/kg, 0.1 to 3 mg/kg, 0.1 to 2 mg/kg, 0.1 to 1 mg/kg, 1 to 50 mg/kg, 1 to 40 mg/kg, 1 to 30 mg/kg, 1 to 20 mg/kg, 1 to 10 mg/kg, 1 to 5 mg/kg, 1 to 4 mg/kg, 1 to 3 mg/kg, 1 to 2 mg/kg, or 1 to 1 mg/kg body weight. It is to be noted that dosage values may vary with the type and severity of the conditions to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Toxicity and therapeutic index of the pharmaceutical compositions of the disclosure can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effective dose is the therapeutic index and it can be expressed as the ratio LD₅₀/ED₅₀. Compositions that exhibit large therapeutic indices are generally preferred.

The dosing frequency of the administration of the VitoKine construct pharmaceutical composition depends on the nature of the therapy and the particular disease being treated. The subject can be treated at regular intervals, such as weekly or monthly, until a desired therapeutic result is achieved. Exemplary dosing frequencies include but are not limited to: once weekly without break; once weekly, every other week; once every 2 weeks; once every 3 weeks; weakly without break for 2 weeks, then monthly; weakly without break for 3 weeks, then monthly; monthly; once every other month; once every three months; once every four months; once every five months; or once every six months, or yearly.

### Combination Therapy

The subsequent paragraphs of this sub-section “Combination Therapy” will utilize PD1-targeted IL-2 VitoKine (VitoKine) constructs as illustrative instances, yet these concepts shall equally be applicable to the PD1-targeted IL-2 immunocytokines of the current invention.

As used herein, the terms “co-administration”, “co-administered” and “in combination with”, referring to a VitoKine construct of the disclosure and one or more other therapeutic agents, is intended to mean, and does refer to and include the following: simultaneous administration of such combination of a VitoKine construct of the disclosure and therapeutic agent(s) to a subject in need of treatment, when such components are formulated together into a single dosage form which releases said components at substantially the same time to said subject; substantially simultaneous administration of such combination of a VitoKine construct of the disclosure and therapeutic agent(s) to a subject in need of treatment, when such components are formulated apart from each other into separate dosage forms which are taken at substantially the same time by said subject, whereupon said components are released at substantially the same time to said subject; sequential administration of such combination of a VitoKine construct of the disclosure and therapeutic agent(s) to a subject in need of treatment, when such components are formulated apart from each other into separate dosage forms which are taken at consecutive times by said subject with a significant time interval between each administration, whereupon said components are released at substantially different times to said subject; and sequential administration of such combination of a VitoKine construct of the disclosure and therapeutic agent(s) to a subject in need of treatment, when such components are formulated together into a single dosage form which releases said components in a controlled manner whereupon they are concurrently, consecutively, and/or overlappingly released at the same and/or different times to said subject, where each part may be administered by either the same or a different route.

In another aspect, the present disclosure provides a method for treating cancer or cancer metastasis in a subject, comprising administering a therapeutically effective amount of the pharmaceutical compositions of the invention in combination with a second therapy, including, but not limited to immunotherapy, cytotoxic chemotherapy, small molecule kinase inhibitor targeted therapy, surgery, radiation therapy, and stem cell transplantation. For example, such methods can be used in prophylactic cancer prevention, prevention of cancer recurrence and metastases after surgery, and as an adjuvant of other conventional cancer therapy. The present disclosure recognizes that the effectiveness of conventional cancer therapies (e.g., chemotherapy, radiation therapy, phototherapy, immunotherapy, and surgery) can be enhanced through the use of the combination methods described herein.

A wide array of conventional compounds has been shown to have anti-neoplastic activities. These compounds have been used as pharmaceutical agents in chemotherapy to shrink solid tumors, prevent metastases and further growth, or decrease the number of malignant T-cells in leukemic or bone marrow malignancies. Although chemotherapy has been effective in treating various types of malignancies, many anti-neoplastic compounds induce undesirable side effects. It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

In various embodiments, a second anti-cancer agent, such as a chemotherapeutic agent, will be administered to the patient. The list of exemplary chemotherapeutic agent includes, but is not limited to, daunorubicin, dactinomycin, doxorubicin, bleomycin, mitomycin, nitrogen mustard, chlorambucil, melphalan, cyclophosphamide, 6-mercaptopurine, 6-thioguanine, bendamustine, cytarabine (CA), 5-fluorouracil (5-FU), floxuridine (5-FUdR), methotrexate (MTX), colchicine, vincristine, vinblastine, etoposide, teniposide, cisplatin, carboplatin, oxaliplatin, pentostatin, cladribine, cytarabine, gemcitabine, pralatrexate, mitoxantrone, diethylstilbestrol (DES), fluradabine, ifosfamide, hydroxyureataxanes (such as paclitaxel and doxetaxel) and/or anthracycline antibiotics, as well as combinations of agents such as, but not limited to, DA-EPOCH, CHOP, CVP or FOLFOX. In various embodiments, the dosages of such chemotherapeutic agents include, but is not limited to, about any of 10 mg/m², 20 mg/m², 30 mg/m², 40 mg/m², 50 mg/m², 60 mg/m², 75 mg/m², 80 mg/m², 90 mg/m², 100 mg/m², 120 mg/m², 150 mg/m², 175 mg/m², 200 mg/m², 210 mg/m², 220 mg/m², 230 mg/m², 240 mg/m², 250 mg/m², 260 mg/m², and 300 mg/m².

In various embodiments, the combination therapy methods of the present disclosure may further comprise administering to the subject a therapeutically effective amount of immunotherapy, including, but are not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints), such as including, but not limited to antibody to CTLA-4, PDL-1, CD40, OX-40, CD137, GITR, LAG3, TIM-3, SIRPα, CD47, GITR, ICOS, CD27, Siglec 7, Siglec 8, Siglec 9, Siglec 15, VISTA, CD276, CD272, TIM-3, and B7-H4; treatment using bispecific T cell engaging antibodies (BiTE®) such as blinatumomab; treatment involving administration of biological response modifiers such as IL-7, IL-10, IL-12, IL-15, IL-21, IL-22, GM-CSF, IFN-α, IFN-β, IFN-γ, TGF-β antagonist or TGF-β trap; treatment using therapeutic vaccines, including, but not limited to oncolytic virus, such as T-vec, or therapeutic vaccine, such as sipuleucel-T; treatment using dendritic cell vaccines, or tumor antigen peptide or neoantigen vaccines; treatment using chimeric antigen receptor (CAR)-T cells; treatment using CAR-NK cells; treatment using NK cell; treatment using iPS induced-NK cells; treatment using iPS induced-T cells; treatment using iPS induced CAR-T or iPS induced CAR-NK cells treatment using tumor-infiltrating lymphocytes (TILs); treatment using adoptively transferred anti-tumor T cells (ex vivo expanded and/or TCR-T cells); treatment using TALL-104 cells; and treatment using immunostimulatory agents such as Toll-like receptor (TLR) agonists CpG, TLR7,TLR8, TLR9, and vaccine such as Bacille Calmette-Guerine (BCG), and imiquimod; wherein the combination therapy provides increased effector cell killing of tumor cells, i.e., a synergy exists between the VitoKine construct and the immunotherapy when co-administered.

In various embodiments, the combination therapy comprises administering a VitoKine construct and the second agent composition simultaneously, either in the same pharmaceutical composition or in separate pharmaceutical composition. In various embodiments, a VitoKine construct composition and the second agent composition are administered sequentially, i.e., a VitoKine construct composition is administered either prior to or after the administration of the second agent composition. In various embodiments, the administrations of a VitoKine construct composition and the second agent composition are concurrent, i.e., the administration period of a VitoKine construct composition and the second agent composition overlap with each other. In various embodiments, the administrations of a VitoKine construct composition and the second agent composition are non-concurrent. For example, in various embodiments, the administration of a VitoKine construct composition is terminated before the second agent composition is administered. In various embodiments, the administration of a second agent composition is terminated before a VitoKine construct composition is administered.

The following examples are offered to more fully illustrate the disclosure but are not construed as limiting the scope thereof.

### Example 1

### Sequence Optimization of the Pembrolizumab Variable Domains

The current invention aims to optimize the variable domain sequences of pembrolizumab to enhance the score of similarity to the human germline sequences, a measure for their “humanness”. This enhancement could potentially lower the immunogenicity risk. Additionally, the inventors used human VH3 family germline sequence, which, despite being less homologous to pembrolizumab, is more prevalent and behave better, as an alternative acceptor framework. This was done with the aim of improving the biophysical properties of the resulting humanized antibody, ensuring it retains full activity, and enhancing its sequence humanness.

Pembrolizumab was humanized by CDR grafting technology using the most homologous human antibody sequences available in RCSB protein databank as the acceptor human frameworks. The frameworks found in GenBank under accession numbers AB063829 (SEQ ID NO: 40) and M29469 (SEQ ID NO: 41) were used as the acceptor human frameworks for the heavy chain variable domain (VH) and light chain variable domain (VL), respectively (Carven G J et al., U.S. Pat. No. 8,354,509B2). However, pembrolizumab only shares 79.6% sequence identity to IGHV1-2, its closest human germline, according to the comparison of the variable region exons using the International Immunogenetics Information System (IMGT) DomainGapAlign tool (www.imgt.org). A similarity score to the human germline sequence was proposed as a defining criterion for therapeutic antibodies by the international nonproprietary names (INN) group of WHO in 2014, presumably based on the notion that higher similarity could suggest reduced immunogenicity. The low similarity score, or “degree of humanness” (Abhinandan K R et al., J Mol Biol (2007) 369:852-62) of pembrolizumab's heavy chain could be due to the poor level of conservation between the mouse CDRs and their human sequence equivalents, the necessity to retain a few structurally important mouse framework residues to recapitulate antigen binding, and the preservation of unique somatic mutations in the human framework sequence AB063829.

To enhance the degree of humanness of pembrolizumab, certain CDR residues were targeted for substitution with their equivalent residues from the closest human germline sequences. This method is herein referred to as CDR germlining. While avoidance of CDR perturbation has traditionally been a central principle humanized Abs design, only a limited number of CDR residues engage in direct antigen interaction. Therefore, certain CDR residues may be replaced without compromising the activity of the antibody. According to the Kabat numbering scheme, CDRs are defined as amino acid residues 24-34 (CDR-L1), 50-56 (CDR-L2), 89-97 (CDR-L3), 31-35b (CDR-H1), 50-65 (CDR-H2), and 95-102 (CDR-H3).

As for the CDR3 sequences, a portion of the light chain CDR3 (CDR-L3) and the entire heavy chain CDR3 (CDR-H3) were not part of the germline sequence's variable region exons V region. Consequently, there are no human germline residues available to substitute the mouse CDR counterparts. Additionally, CDR3s, especially CDR-H3, are highly variable and vital for antigen binding and functional activity, making it crucial to preserve their conformations. As such, both CDR-L3 (QHSRDLPLT; SEQ ID NO: 25) and CDR-H3 (RDYRFDMGFDY; SEQ ID NO: 33) of pembrolizumab were excluded from the CDR germlining process.

The sequences of pembrolizumab's CDR-L1 and CDR-L2 were aligned to their counterparts from the closest human germline IGKV3D-11 (GenBank accession #X17264; SEQ ID NO: 39). The alignments are shown in Table 6A. Likewise, the alignments of CDR-H1 and CDR-H2 sequences of pembrolizumab with the closest human germline sequence, IGHV1-2 (GenBank accession #X62106; SEQ ID NO: 37), are displayed in Table 6B.

TABLE 6A
Alignments of the CDR-L1 and CDR-L2 sequences
of pembrolizumab with IGKV3D-11

| Antibody | CDR-L1 | CDR-L2 |
|---|---|---|
| Pembrolizumab | RASKGVSTSGYSYLH | LASYLES |
| IGKV3D-11 | RASQGV----SSYLA | DASNRAT |

TABLE 6B
Alignments of the CDR-H1 and CDR-H2
sequences of pembrolizumab with IGHV1-2

| Antibody | CDR-H1 | CDR-H2 |
|---|---|---|
| Pembrolizumab | NYYMY | GINPSNGGTNFNEKFKN |
| IGHV1-2 | GYYMH | WINPNSGGTNYAQKFQG |

“-” indicates sequence gap.
Residues in bold and italic represent those interacting with PD1, as per the complex structure (Horita S. et al., Sci Rep (2016) 6:35297).
Underscored residues are subject to the CDR germlining process.

Multiple pembrolizumab CDR residues directly engage polar interactions with PD1, e.g., hydrogen bond and salt bridge (Horita, S. et al. Sci. Rep (2016). 6: 35297). The contacting residues in or around both VL and VH CDR1 and CDR2 include ^{L}Ser28, ^{L}Tyr30 in CDR-L1, ^{L}Tyr49 immediately prior to CDR-L2, ^{L}Tyr53 in CDR-L2, ^{H}Tyr33, ^{H}Tyr35 in CDR-H1, ^{H}Asn52, ^{H}Ser53, ^{H}Asn54, ^{H}Thr57, ^{H}Asn58 in CDR-H2 (where the superscripted letter ‘L’ donates light chain, and ‘H’ refers to heavy chain). Except for ^{L}Tyr49, which is not a CDR residue and not shown, all the antigen-interacting CDR residues mentioned above are in bold and italic in Tables 6A and 6B. Among the CDR residues that differ from the germline sequences, CDR-L1 residues ^{L}Lys27, ^{L}His34, CDR-L2 residues ^{L}Leu54, ^{L}Glu55, CDR-H2 residues ^{H}Phe59, ^{H}Asn60, ^{H}Glu61, ^{H}Lys64, and ^{H}Asn65 (underscored in Tables 6A and 6B) were selected for CDR germlining. They were replaced with their respective human germline equivalents with the following amino acid substitutions: ^{L}K27Q, ^{L}H34A, ^{L}L54R, ^{L}E55A, ^{H}F59Y, ^{H}N60A, ^{H}E61Q, ^{H}K64Q, and ^{H}N65G either individually or in combination. Other CDR residues were reserved to avoid any disruption of the antigen-interacting residues.

For CDR-H1 (NYYMY; SEQ ID NO: 26), only a single residue, ^{H}Asn31, is eligible for CDR germlining, to be replaced with its equivalent residue, glycine, in the human germline sequence. Other residues were kept either because they engage in direct antigen interaction with PD1 (^{H}Tyr33 and ^{H}Tyr35) or because they are conserved between the mouse and human germline sequences (^{H}Tyr32 and ^{H}Met34). However, considering CDR-H1's short length of only five amino acids and the fact that two residues have a direct interaction with the antigen, any amino acid alteration could potentially disturb the CDR confirmation and impact the antibody's activity. Consequently, ^{H}Asn31 was not modified through CDR germlining, and CDR-H1 is fully reserved.

In addition to the low level of conservation between the mouse CDRs and their corresponding human germline sequences, the pembrolizumab heavy chain frameworks (FRs) also contain multiple non-germline residues. They arose due to the retention of the unique somatic mutations in the acceptor framework sequence AB063829. These somatic mutations, including ^{H}Val9 in FR-H1, ^{H}Thr76, ^{H}Lys82a, ^{H}Gln83, ^{H}Phe84 in FR-H3 and ^{H}Thr108 in FR-H4, are not considered structurally important. Replacement with their respective germline counterparts, ^{H}V9A, ^{H}T76S, ^{H}K82aS, ^{H}Q83R, ^{H}F84S, ^{H}T108L, leads to a considerable improvement in the similarity score to the human germline sequence, without disturbing the CDR conformation or altering the antibody's activity.

Moreover, IGHV3-23 (SEQ ID NO: 38) was used as an alternative acceptor framework to investigate whether utilizing a human acceptor framework with substantially lower sequence homology, but superior biophysical attributes could enhance the biophysical properties of the resultant humanized antibody without compromising its functional activity. IGHV3-23 belongs to the human antibody heavy chain germline VH3 family, which is the most common VH family in the human repertoire. It is also the most prevalent among the marketed human monoclonal antibodies and is widely acknowledged for its superior drug-like properties. Given that the CDR conformation was exquisitely sensitive to the chemical environment of the surrounding framework, a few structurally significant framework residues in pembrolizumab, which differ from their VH3 germline family equivalents, were selected for reverse mutation to their corresponding pembrolizumab equivalents. Furthermore, several CDR-H2 residues were targeted for CDR germlining using IGHV3-23 CDR-H2 as a template to improve the similarity score to the human germline sequence.

Alignments of the CDR-H1 and CDR-H2 sequences of pembrolizumab with IGHV3-23 are displayed in Table 6C. Six CDR-H2 residues, ^{H}Phe59, ^{H}Asn60, ^{H}Glu61, ^{H}Lys62, ^{H}Phe63, and ^{H}Asn65 (underscored in Table 6C; the superscripted letter ‘L’ donates light chain, and ‘H’ refers to heavy chain) were selected for CDR germlining with the following amino acid substitutions: ^{H}F59Y, ^{H}N60A, ^{H}E61 D, ^{H}K62S, ^{H}F63V, and ^{H}N65G. CDR-H1 was excluded from the CDR germlining process for the reason described earlier.

TABLE 6C
Alignments of the CDR-H1 and CDR-H2 sequences
of pembrolizumab with IGHV3-23

| Antibody | CDR-H1 | CDR-H2 |
|---|---|---|
| Pembrolizumab | NYYMY | GINPSNGGTNFNEKFKN |
| IGHV3-23 | SYAMS | AISGSGGSTYYADSVKG |

Residues in bold and italic represent those interacting with PD1, as per the complex structure (Horita S. et al., Sci Rep (2016) 6:35297).
Underscored residues are subject to the CDR germlining process

Two framework residues, ^{H}Thr30 and ^{H}Arg94, were considered structurally significant and were preserved without alteration to their corresponding germline equivalents, ^{H}Ser30 and ^{H}Lys94. Five additional IGHV3 framework residues, ^{H}Val48, ^{H}Ser49, ^{H}Ile69, ^{H}Arg71, and ^{H}Asn73, which belong to the vernier zone (U.S. Pat. Nos. 5,821,337 and 5,859,205) and may be of structural significance, were reverted to their corresponding pembrolizumab residues, ^{H}Met48, ^{H}Gly49, ^{H}Leu69, ^{H}Thr71, and ^{H}Ser73, either individually or in combination. The importance of specific framework amino acid residues was assessed experimentally. The number of reverse mutations was minimized to ensure the highest similarity score to the germline sequence without negatively affecting antibody activity.

All the optimized antibody sequences were expressed as full length antibody with a kappa light chain constant region containing the sequence set forth in SEQ ID NO: 34 and a modified IgG1 heavy chain constant region containing the sequence set forth in SEQ ID NO: 35. Table 7 lists the SEQ ID NOS of the VL, VH, CDR-L1, CDR-L2, and CDR-H2 of the exemplary optimized PD1 blocking antibodies along with the Reference Antibody (P-0734), comprising VL and VH sequences set forth in SEQ ID NO: 2 and SEQ ID NO: 6, respectively. All antibodies of the present invention comprise identical CDR-L3 (SEQ ID NO: 25), CDR-H1 (SEQ ID NO: 26), and CDR-H3 (SEQ ID NO: 33).

TABLE 7
Exemplary PD1 blocking antibodies
resulted from CDR and FR germlining

| PD1 | SEQ ID NO | | | | |
|---|---|---|---|---|---|
| Antibody | VL | VH | CDR-L1 | CDR-L2 | CDR-H2 |
| P-0734 | 2 | 6 | 19 | 22 | 27 |
| P-1127 | 3 | 6 | 20 | 23 | 27 |
| P-1129 | 4 | 6 | 20 | 24 | 27 |
| P-1148 | 4 | 7 | 20 | 24 | 28 |
| P-1150 | 5 | 7 | 21 | 24 | 28 |
| P-1151 | 4 | 8 | 20 | 24 | 29 |
| P-1153 | 5 | 8 | 21 | 24 | 29 |
| P-1174 | 3 | 7 | 20 | 23 | 28 |
| P-1175 | 2 | 10 | 19 | 22 | 31 |
| P-1176 | 2 | 11 | 19 | 22 | 31 |
| P-1177 | 2 | 12 | 19 | 22 | 31 |
| P-1178 | 2 | 13 | 19 | 22 | 31 |
| P-1181 | 2 | 14 | 19 | 22 | 30 |
| P-1193 | 3 | 15 | 20 | 23 | 32 |
| P-1194 | 3 | 11 | 20 | 23 | 31 |
| P-1198 | 3 | 16 | 20 | 23 | 32 |
| P-1199 | 3 | 17 | 20 | 23 | 32 |
| P-1201 | 3 | 18 | 20 | 23 | 32 |
| P-1238 | 3 | 13 | 20 | 23 | 32 |
| P-1271 | 3 | 9 | 20 | 23 | 28 |

### Example 2

### Construction, Production, and Purification of the Optimized PD1 Blocking Antibodies

All genes were codon optimized for expression in mammalian cells, and they were synthesized and subsequently subcloned into their recipient mammalian expression vectors through the service of GenScript. Protein expression is driven by a CMV promoter, and a synthetic SV40 polyA signal sequence is positioned at the 3′ end of the coding sequence. A leader sequence was engineered at the N-terminus of the constructs to ensure appropriate signaling and processing for secretion.

The antibodies were produced by co-transfecting vectors harboring light chain and heavy chain with a 1:1 ratio in ExpiCHO cells (ThermoFisher) following the manufacturer's instructions. On the day of transfection, ExpiCHO cells were diluted to 6×10⁶ cells/mL in ExpiCHO™ expression medium (ThermoFisher). Expression vectors, totaling 0.8 μg DNA/mL culture volume, were mixed with cold OptiPRO™ medium (40 μL/mL cell culture). Following the addition of ExpiFectamine™ CHO reagent at 3.2 μL/mL cell culture, the solution was gently mixed and subsequently incubated for 5 min at room temperature. The ExpiFectamine™ CHO/plasmid DNA complexes were then slowly transferred to the cells and incubated at 37 °C in a shaker incubator at 130 rpm with 8% CO₂ atmosphere. ExpiFectamine™ CHO enhancer (6 μL/mL cell culture) and ExpiCHO™ feed (240 μL/mL cell culture) were added to the flask with gentle swirling 18-22 hours post transfection. After 8 days of cultivation, the supernatant was harvested for purification by centrifugation for 20 min at 2200 rpm, followed by sterile filtered using a 0.22 μm filter (Corning).

The secreted antibody was purified from cell culture supernatants using Protein A affinity chromatography. Cell culture supernatant was loaded onto a MabSelect SuRe 5-mL column (Cytiva) equilibrated with 5 column volumes (CV) of phosphate buffered saline, pH 7.2 (ThermoFisher). Unbound protein was removed by washing with 5 CVs of PBS, pH 7.2, and target protein was eluted with 25 mM sodium citrate, 25 mM sodium chloride buffer, pH 3.2. Antibody solution was neutralized by adding 3% of 1 M Tris buffer, pH 10.2 followed by concentration and buffer exchange to PBS, pH 7.2 using Amicon®Ultra-15 Ultracel with 10 KDa MWCO (Merck Millipore).

The purity and molecular weight of the purified antibodies were analyzed by SDS-PAGE, both with and without a reducing agent, and then stained with Coomassie (Imperial™ protein stain, ThermoFisher). The SurePAGE™ Pre-Cast gel system (8-16% Bis-Tris, GenScript) was used according to the manufacturer's instruction. The aggregate content of the antibodies was analyzed on an Agilent 1200 high-performance liquid chromatography (HPLC) system. Samples were injected into an AdvanceBio size-exclusion column (300A, 4.6×150 mm, 2.7 μm, LC column, Agilent) using 150 mM sodium phosphate buffer, pH 7.0 as the mobile phase at 25 °C.

The antibody concentration of purified protein samples was determined by measuring the absorbance at 280 nm using a Nanodrop spectrophotometer (ThermoFisher) divided by the molar extinction coefficient calculated based on its amino acid sequence. Endotoxin level of purified protein samples were measured using Endosafe nexgen-PTS (Charles River) as per the manufacturer's instruction.

### Example 3

### Assays to Evaluate the Biological Activities of the Optimized PD1 Blocking Antibodies

Antibodies of the invention were tested for their antigen binding activity by well-known methods such as enzyme-linked immunosorbent assay (ELISA). Briefly, Nunc Maxisorp plates (ThermoFisher) were coated with recombinant human PD1 protein in bicarbonate buffer, pH 9.4 (ThermoFisher), overnight at 4 °C, using 1 μg of antigen per well (100 μL/well). After triple washing with PBS/0.05% Tween 20, plates were incubated with SuperBlock (ThermoFisher) for two hours at room temperature to block nonspecific binding. The PD1 antibodies, serially diluted three-fold with blocking buffer (PBS with 1% bovine serum albumin) were added to the plates (100 μL/well) post washing and incubated at room temperature for one hour. After another washing, antibodies were detected by incubating with a horseradish peroxidase (HRP)-conjugated goat anti-human IgG Fc antibody (ThermoFisher) diluted 1:5000 in blocking buffer (100 μL/well) for one hour at room temperature. After a final wash, TMB substrate (ThermoFisher) at 100 μL/well was added. Plates were sealed and left to incubate in dark for 5-20 minutes. The reaction was stopped by adding 2N sulfuric acid (Ricca Chemical) (50 uL/well), and the absorbance was measured at 450 nm with a plate reader. The curves were plotted, and the half-maximal effective concentration (EC₅₀) values were calculated using GraphPad Prism software.

Additionally, HEK 293T cells stably expressing human PD1 gene (Crown Bioscience) was used to determine the cell-based binding strength of the optimized PD1 antibodies by flow cytometry. After harvesting, HEK293-hPD1 cells were seeded into a 96-well U-bottom plate at 1×10⁵ cells/well (100 μL), incubated with Fc block (1:50) for 20 minutes at 4 °C, and subsequently washed with FACS buffer (PBS, 1% FBS). Cells were then treated with three-fold serial dilutions of each antibody at concentrations ranging from 0.01-100 nM in FACS buffer for 30 minutes at 4 °C. Subsequently, cells were washed twice with FACS buffer to remove unbound molecules, and 40 μL of the 1:100 diluted PE-labeled goat anti-human Fc secondary antibody (eBiosciences) was added to the cells. Following a 30-minute incubation at 4 °C and another double wash with FACS buffer, antibodies bound to the cells were detected with PE-labeled secondary antibody by flow cytometry (BD ACCURI-C6), and EC₅₀ values were calculated using GraphPad Prism software.

Furthermore, a thaw-and-use format of Promega luciferase reporter assay, a biologically relevant mechanistic-based assay, was used to measure the potency in blocking PD1 interaction. Cell thawing and plating procedures were followed exactly as described in the manufacturer's protocol.

Briefly, one vial (0.5 mL) of PD-L1 aAPC/CHO-K1 cells were thawed and mixed with 14.5 mL cell recovery medium (90% Ham's F12/10% FBS). Next, 100 μL of this cell suspension was added to the inner 60 wells of two 96-well flat-bottom assay plates, while perimeter wells received 100 μL of cell recovery medium. After overnight incubation at 37 °C and 5% CO2, the medium was discarded. The inner wells received 40 μL of 3-fold serially diluted compounds, while the perimeter wells got 80 μL of assay buffer (99% RPMI 1640/1% FBS). Subsequently, one vial (0.5 mL) of PD1 effector cells were thawed and mixed with 5.9 mL of assay buffer, and 40 μL of this mixture was added to the inner wells. After a 6-hour incubation at 37 °C, 5% CO2, and a 7-minute equilibration at room temperature, 80 μL of Bio-Glo™ reagent was added to all wells. The plates were then incubated at room temperature for 10 minutes with shaking. The resulting luminescence was measured using a luminescence plate reader (BioTek synergy h1).

Background was calculated by averaging relative light units (RLU) of perimeter wells. Fold Induction was determined as the RLU of the antibody sample minus background, divided by the RLU of the control samples (without antibody) minus background, or fold induction=RLU (antibody-background))/(RLU (no antibody ctrl-background). Finally, EC₅₀ values were determined using the curves fitted using GraphPad Prism software.

### Example 4

### Evaluation of the PD1 Antibodies Comprising Germlining Modifications Based on the Closest Human Germline Sequences

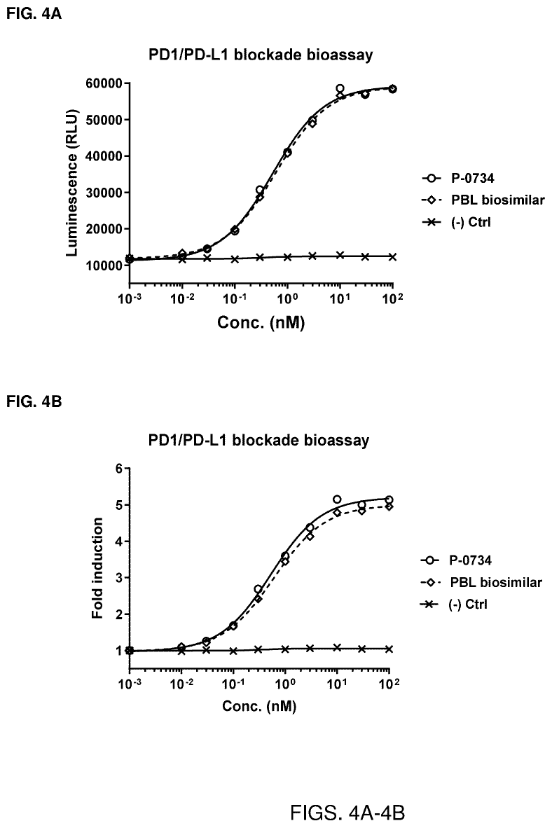

Firstly, the effectiveness of P-0734 in inhibiting the PD1/PD-L1 interaction was compared with that of the pembrolizumab (PBL) biosimilar. While P-0734 and PBL biosimilar share the identical variable domains, they differ in their heavy chain constant region. PBL contains an IgG4 constant chain containing the S228P mutation (SEQ ID NO: 36), whereas P-0734 has an IgG1 constant chain with L234A/L235A/G237A mutations (SEQ ID NO: 35) to abrogate Fc effector functions. As shown in FIG. 4, P-0734 and PBL biosimilar were equally potent in blocking the interaction between PD1 and PD-L1. This result was expected and confirms that the ability to block PD1 is determined by the variable domain sequences, and not by the immunoglobulin classes. P-0734 faithfully recapitulates the potency of PBL biosimilar in blocking the PD1/PD-L1 interaction and is herein referred to as the Reference Antibody.

The impacts of CDR germlining substitutions ^{L}H34A in CDR-L1 and HF60Y in CDR-H2 were evaluated using antibodies with slightly different mutational contexts. These antibodies, P-1148, P-1150, P-1151, and P-1153, all contain germlining substitutions ^{L}K27Q in CDR-L1, ^{L}L54R, ^{L}E55A in CDR-L2, and ^{H}N60A, ^{H}E61Q, ^{H}K64Q, ^{H}N65G in CDR-H2. P-1150 includes an additional ^{L}H34A substitution in CDR-L1, P-1151 has an extra ^{H}F59Y substitutions in CDR-H2, and P-1153 harbors both ^{L}H34A and ^{H}F59Y changes in addition. Table 8 provides a list of the CDR germlining substitutions for these exemplary PD1 blocking antibodies.

TABLE 8
CDR germlining substitutions of exemplary PD1 blocking antibodies

| PD1 Ab | CDR-L1 | CDR-L2 | CHR-H2 |
|---|---|---|---|
| P-1148 | ^{L}K27Q | ^{L}L54R, ^{L}E55A | ^{H}N60A, ^{H}E61Q, ^{H}K64Q, ^{H}N65G |
| P-1150 | ^{L}K27Q, ^{L}H34A | ^{L}L54R, ^{L}E55A | ^{H}N60A, ^{H}E61Q, ^{H}K64Q, ^{H}N65G |
| P-1151 | ^{L}K27Q | ^{L}L54R, ^{L}E55A | ^{H}F59Y, ^{H}N60A, ^{H}E61Q, ^{H}K64Q, ^{H}N65G |
| P-1153 | ^{L}K27Q, ^{L}H34A | ^{L}L54R, ^{L}E55A | ^{H}F59Y, ^{H}N60A, ^{H}E61Q, ^{H}K64Q, ^{H}N65G |

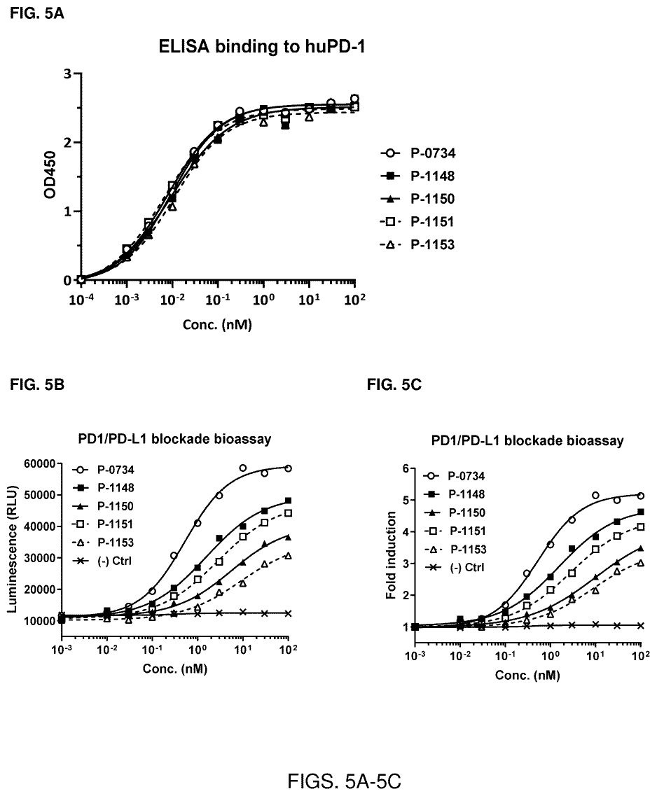

As illustrated in FIGS. 5B & 5C and summarized in Table 9, the CDR-L1 germlining substitution ^{L}H34A consistently led to a reduction in PD1 blockade potency (EC₅₀) of approximately 3.5-fold, along with a 25% reduction in both Eₘₐₓ (maximum effect/luminescence signal) and fold induction, regardless of the presence (P-1151 vs P-1153) or absence (P-1148 vs P-1150) of the ^{H}F59Y substitution. The impact of ^{H}F59Y germlining substitution was similarly assessed, with the data shown in FIGS. 5B & 5C and summarized in Table 9. Irrespective of whether the ^{L}H34A substitution is present (P-1150 vs P-1153) or absent (P-1148 vs P-1151), the ^{H}F59Y CDR germlining substitution resulted in a consistent albeit modest reduction in both potency (EC₅₀; reduced by about 1.8-fold) and signal (10-15% decrease in both Eₘₐₓ and fold induction). Compared to P-0734, the cumulative CDR germlining substitutions in P-1153 ended up with a nearly 20-fold decrease in PD1 blockade potency (EC₅₀) and 50% reduction in Eₘₐₓ. As a result, both ^{L}H34A and ^{H}F59Y substitutions were deemed detrimental in this particular framework and the original CDR residues, ^{L}His34 and ^{H}Phe59, will be preserved.

However, despite the notable differences in potency in blocking the PD1/PD-L1 interaction, all four optimized PD1 antibodies and the Reference Antibody, P-0734, displayed nearly identical binding strength with an EC₅₀ value close to 100 pM (FIG. 5A and Table 9).

TABLE 9
ELISA binding and PD1 blocking activity
of exemplary PD1 blocking antibodies

| | | PD1 blocking activity (reporter assay) | | |
|---|---|---|---|---|
| | ELISA binding | | | Fold |
| PD1 Ab | EC₅₀ (pM) | EC₅₀ (nM) | Vmax (RLU) | Induction |
| P-0734 | 82 | 0.53 | 58381 | 5.13 |
| P-1148 | 93 | 1.56 | 48227 | 4.63 |
| P-1150 | 88 | 5.55 | 36537 | 3.49 |
| P-1151 | 65 | 2.83 | 44252 | 4.15 |
| P-1153 | 110 | 9.88 | 30760 | 3.04 |

This piece of data suggested that the mechanistic-based functional assay was capable of discerning minute activity changes that aren't detectable by ELISA binding assay. As such, the luciferase PD1/PD-L1 reporter assay is herein used as the primary tool to characterize and rank PD1 blocking antibodies derived from pembrolizumab via germlining substitutions. It is expected that the derivative antibodies that maintain full functional activity will exhibit identical in vivo efficacy as pembrolizumab.

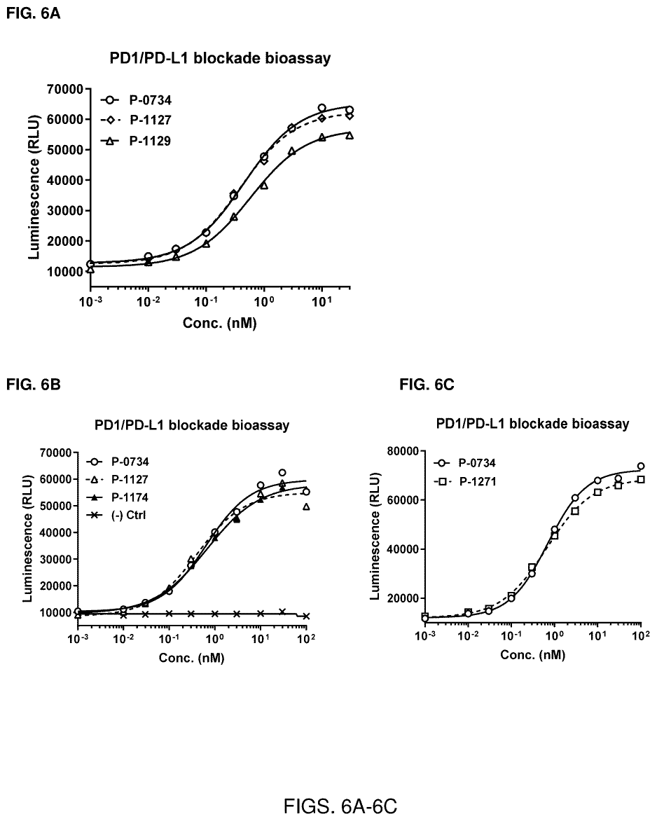

The potential negative effects of CDR-L2 germlining substitution ^{L}E55A was further assessed by comparing P-1127 and P-1129. Both these molecules contain ^{L}K27Q and ^{L}K54E germline substitutions in the light chain CDRs, with the only sequence difference being the extra CDR-L2 substitution, ^{L}E55A, in P-1129. As demonstrated in FIG. 6A, P-1129 displayed a minor yet appreciable reduction in potency (EC₅₀=0.39 nM and 0.58 nM for P-1127 and P-1129, respectively) and a marginal 10% decrease in Eₘₐₓ. Hence, the^{L}E55A amino acid substitution was deemed adverse and the original residue, ^{L}Glu55, will be preserved.

P-1174, harboring a total of 6 CDR germlining substitutions, ^{L}K27Q, ^{L}L54R, ^{H}N60A, ^{H}E61Q, ^{H}K64Q, and ^{H}N65G, exhibited identical PD1 blockade activity as P-1127 and P-0734 with EC₅₀ of 0.64 nM, 0.54 nM, and 0.67 nM for P-0734, P-1127, and P-1174, respectively (FIG. 6B). Additionally, P-1174 was derived from P-114 by eliminating one CDR germlining substitution, ^{L}E55A. When compared to P-0734, P-1174 exhibited higher potency than P-1148 (refer to FIG. 5B & FIG. 6B). This piece of data further collaborated the conclusion that the original CDR residues, ^{L}Glu55, should not be altered.

Besides the low conservation between the mouse CDRs and their human germline counterparts, multiple non-germline residues in the pembrolizumab VH framework also contributed to the low sequence similarity score with the germline. These non-germline residues, originating from the unique somatic mutations preserved in the acceptor framework sequence, including ^{H}Val9 in FR-1, ^{H}Thr76, ^{H}Lys82a, ^{H}Gln83, ^{H}Phe84 in FR-3 and ^{H}Thr108 in FR-4, are considered not to be structurally significant. To further enhance the sequence similarity score with the germline or degree of humanness, these non-germline residues in the P-1174 framework were replaced with their respective germline equivalents, ^{H}V9A, ^{H}T76S, ^{H}K82aS, ^{H}Q83R, ^{H}F84S, ^{H}T108L, resulting in P-1271. As expected, P-1271 displayed the same PD1 blocking activity as the Reference Antibody, P-0734 (FIG. 6C) with EC₅₀ values of 0.66 nM for P-1271 and 0.70 for P-0734, respectively.

In conclusion, CDR germlining substitutions, ^{L}K27Q, ^{L}L54R, ^{H}N60A, ^{H}E61Q, ^{H}K64Q, and ^{H}N65G, in P-1174 and P-1271 enhanced antibody sequence degree of humanness without compromising the potency in blocking the PD1/PD-L1 interaction. Additional six framework germlining substitutions in P-1271 further augmented the score of similarity to the closest human germline sequences. Table 10 lists the germlining substitutions and similarity scores to the closest human germline sequences of P-1174 and P-1271 in comparison to the Reference Antibody, P-0734.

TABLE 10
Germlining substitutions and similarity scores of the exemplary optimized PD1
antibodies, P-1174 and P-1271, with the closest human germline sequences

| | | | VL similarity | VH similarity |
|---|---|---|---|---|
| | | FR germlining | score to | score to |
| PD1 Ab | CDR germling substitutions | substitutions | IGKV3D-11 | IGHV1-2 |
| P-0734 | N/A | N/A | 85.1% | 79.6% |
| P-1174 | ^{L}K27Q, ^{L}L54R, ^{H}N60A, | N/A | 87.2% | 83.7% |
| | ^{H}E61Q, ^{H}K64Q, and ^{H}N65G | | | |
| P-1271 | ^{L}K27Q, ^{L}L54R, ^{H}N60A, | ^{H}V9A, ^{H}T76S, | 87.2% | 88.8% |
| | ^{H}E61Q, ^{H}K64Q, and ^{H}N65G | ^{H}K82aS, ^{H}Q83R, | | |
| | | ^{H}F84S, ^{H}T108L | | |

### Example 5

### Evaluation of the PD1 Antibodies Comprising Germlining Modifications Based on a More Prevalent Human Germline Family (VH3)

The adoption of framework germlining substitutions based on the human antibody heavy chain germline IGHV3-23 (SEQ ID NO: 38) was investigated to exam whether an antibody framework with substantially lower sequence homology, but superior biophysical properties, could enhance the drug-like properties of the resultant antibody while fully retaining its functional activity. Of the 33 framework germlining substitutions (Table 11A), the importance of the 5 Vernier zone residues, ^{H}V48, ^{H}S49, ^{H}I69, ^{H}R71, and ^{H}N73, were assessed experimentally by reversion mutation to their respective pembrolizumab equivalents, ^{H}V48M, ^{H}S49G, ^{H}I69L, ^{H}R71T, and ^{H}N73S, either individually or in combination. Additionally, six CDR-H2 residues, ^{H}F59Y, ^{H}N60A, ^{H}E61 D, ^{H}K62S, ^{H}F63V, and ^{H}N65G, were selected for CDR germlining substitutions with their corresponding residues in IGHV3-23. Table 11 B provides a summary of the VH3 germlining substitutions in the exemplary antibodies.

TABLE 11A
VH Framework germlining substitutions based on IGHV3-23

| VH FRs | Designation | Sequences |
|---|---|---|
| FR1 (1-30) | P-0734 | QVQLVQSGVEVKKPGASVKVSCKASGYTFT |
| | After Germlining | EVQLLESGGGLVQPGGSLRLSCAASGETFT |
| FR2 (36-49) | P-0734 | WVRQAPGQGLEWMG |
| | After Germlining | WVRQAPGKGLEWVS |
| FR3 (66-94) | P-0734 | RVTLTTDSSTTTAYMELKSLQFDDTAVYYCAR |
| | After Germlining | RFTISRDNSKNTLYLQMNSLRAEDTAVYYCAR |
| FR4 (103-113) | P-0734 | WGQGTTVTVSS |
| | After Germlining | WGQGTLVTVSS |

The bolded and underlined residues represent a total of 33 framework germlining substitutions.

TABLE 11B
VH CDR germlining and FR reversion mutations based on IGHV3-23

| | | No. of FR | |
|---|---|---|---|
| | CHR-H2 germlining | germlining | Reversion mutation |
| PD1 Ab | substitutions | substitutions | of FR residues |
| P-1175 | ^{H}F59Y, ^{H}N60A, ^{H}E61D, ^{H}N65G | 33 | N/A |
| P-1181 | ^{H}E61D, ^{H}K62S, ^{H}F63V, ^{H}N65G | 33 | N/A |
| P-1176/P-1194 | ^{H}F59Y, ^{H}N60A, ^{H}E61D, ^{H}N65G | 30 | ^{H}I69L, ^{H}R71T, ^{H}N73S |
| P-1177 | ^{H}F59Y, ^{H}N60A, ^{H}E61D, ^{H}N65G | 31 | ^{H}V48M, ^{H}S49G |
| P-1178/P-1238 | ^{H}F59Y, ^{H}N60A, ^{H}E61D, ^{H}N65G | 28 | ^{H}V48M, ^{H}S49G, |
| | | | ^{H}I69L, ^{H}R71T, ^{H}N73S |
| P-1193 | ^{H}N60A, ^{H}E61D, ^{H}N65G | 33 | N/A |
| P-1198 | ^{H}N60A, ^{H}E61D, ^{H}N65G | 32 | ^{H}N73S |
| P-1199 | ^{H}N60A, ^{H}E61D, ^{H}N65G | 31 | ^{H}R71T, ^{H}N73S |
| P-1201 | ^{H}N60A, ^{H}E61D, ^{H}N65G | 30 | ^{H}I69L, ^{H}R71T, ^{H}N73S |

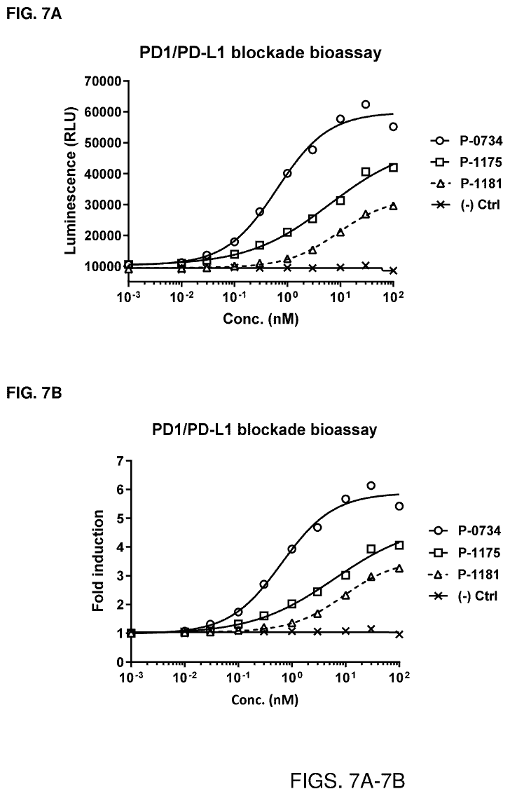

FIG. 7 depicts the PD1 blockade activity of P-1175 and P-1181, differing only in their CDR-H2 germlining substitutions (as shown in Table 11 B). Compared to P-0734, both P-1175 and P-1181 exhibited substantially diminished potency in blocking PD1 interaction. Specifically, P-1174 showed a 10-fold reduction in potency (EC₅₀) and 25% decrease in both Eₘₐₓ and fold induction. This was in comparison to P-1181's 15-fold drop in potency and 40-50% decrease in Eₘₐₓ and fold induction (as illustrated in FIGS. 7A & 7B and summarized in Table 12). Since P-1181 displayed a more drastic decline in activity, its two distinct CDR germlining substitutions, ^{H}K62S ^{H}F63V, were deemed detrimental, hence the original CDR residues, ^{H}Lys62 and ^{H}Phe63, will be preserved. These findings suggested that the significance of individual CDR residues need to be assessed experimentally; even CDR residues that are close to the boundary or are not immediately adjacent to antigen-contacting residues could negatively impact the activity.

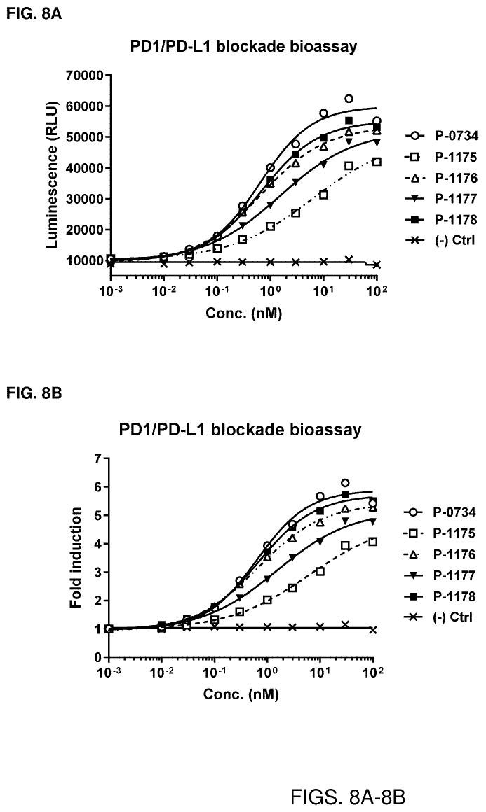

Two to five framework residue reversion mutations were introduced to P-1175, resulting in P-1176, P-1177, and P-1178, as detailed in Table 11. As indicated by the data in FIG. 8, the combined reversion mutations, ^{H}I69L, ^{H}R71T, and ^{H}N73S, in P-1176 effectively restored PD1 blocking activity, almost matching the level of P-0734. Similarly, the activity was significantly reinstated in P-1177 due to the combined reversion mutations, ^{H}V48M and ^{H}S49G, although not as effectively as in P-1176. Nevertheless, the incorporation of these two reversion mutations (^{H}V48M and ^{H}S49G) into P-1176 did not lead to further enhancement in activity for the resulting antibody, P-1178. (P-1178 vs P-1176 in FIG. 8 and Table 12).

TABLE 12
PD1 blockade activity of exemplary PD1 blocking antibodies

PD1 blocking activity (reporter assay)

| PD1 Ab | EC₅₀ (nM) | Vmax (RLU) | Fold induction |
|---|---|---|---|
| P-0734 | 0.64 | 55200 | 5.42 |
| P-1175 | 6.21 | 41970 | 4.07 |
| P-1181 | 9.18 | 29595 | 3.26 |
| P-1176 | 0.67 | 52093 | 5.27 |
| P-1177 | 1.51 | 48120 | 4.77 |
| P-1178 | 0.68 | 53089 | 5.50 |

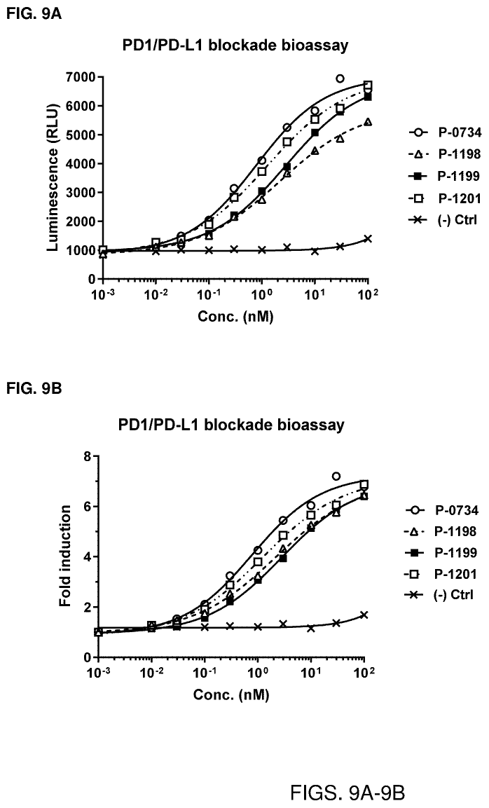

The significance of each of the three FR reversion mutations, ^{H}I69L, ^{H}R71T, and ^{H}N73S were further assessed by comparing PD1 blocking activity of P-1198 (^{H}N73S), P-1199 (^{H}R71T, ^{H}N73S), and P-1201 (^{H}I69L, ^{H}R71T, ^{H}N73S). As demonstrated in FIG. 9, each added reversion mutation led to slight yet evident cumulative increases in PD1 blockade activity. Only the combination of all the three reversion mutations in P-1201 led to nearly fully restored functional activity (with EC₅₀ values of 1.28 nM and 0.78 nM for P-1201 and P-0734, respectively). Thus, all the three reversion mutations, ^{H}I69L, ^{H}R71T, ^{H}N73S, were deemed essential and will be incorporated.

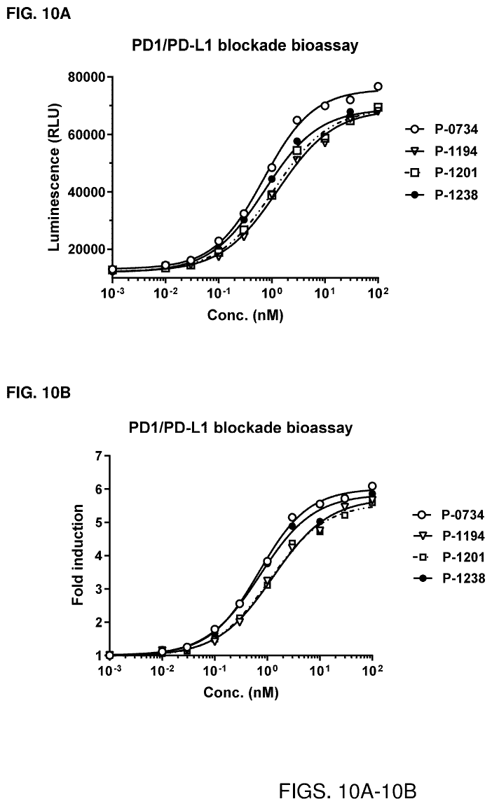

Further, the PD1 inhibitory activity of P-1194, P-1201, and P-1238 were compared and illustrated in FIGS. 10A and 10B. P-1194 and P-1201, differing by only one additional CDR germlining substitution, ^{H}F59Y, displayed identical PD1 blocking potency. This suggests that this particular substitution did not negatively affect the activity, contradicting earlier observation that ^{H}F59Y germlining substitution was detrimental when IGHV1-2 germline sequence was adopted. It is thus postulated that the impact of individual CDR germlining substitution is dependent on the context of the surrounding framework sequences. P-1238 were equally potent as the Reference Antibody, P-0734, with EC₅₀ values of 0.73 nM and 0.70 nM, respectively. Compared to P-1194, the two additional framework reversion mutations, ^{H}V48M, and ^{H}S49G, in P-1238 contributed to slight but discernable improvement in activity.

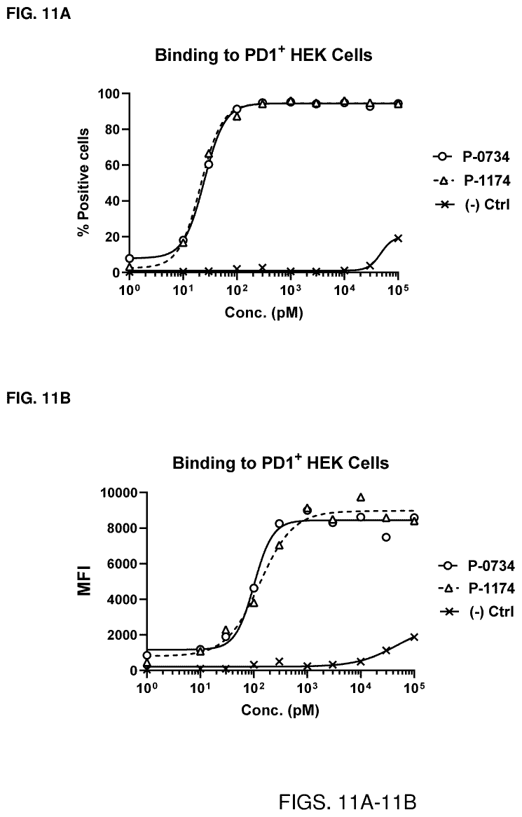

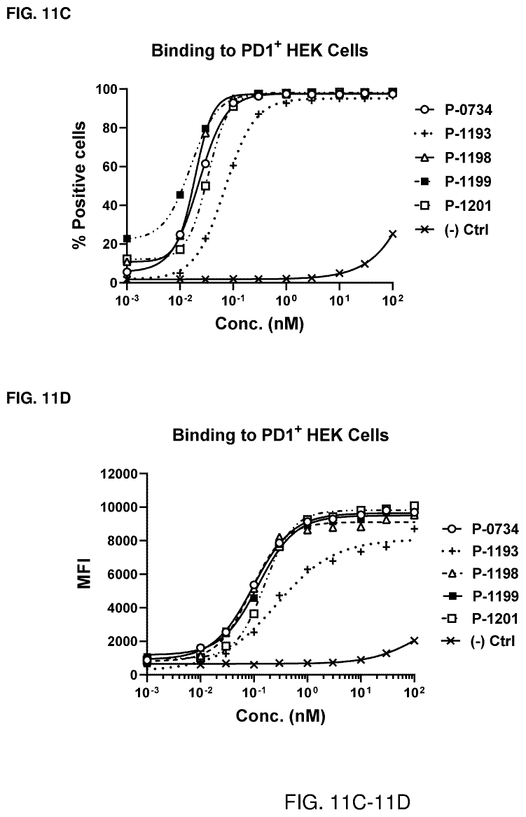

In the final assessment, P-1174, P-1193, P-1198, P-1199, and P-1201 were assessed for their binding strength to PD1+ cells (FIG. 11). As anticipated, P-1174, which fully preserved PD1 blocking potency (FIGS. 6C and 6D), displayed a binding affinity to PD1-expressing cells equivalent to the Reference Antibody, P-0734 (FIGS. 11A and 11B). P-1198, P-1199, and P-1201, which contain 1-3 framework reversion mutations, displayed subtle but evident potency difference in blocking PD1 interaction (FIG. 9), but such variations in activity were not detected in the cell-based binding assay. All three compounds demonstrated an equal ability in binding to PD1+ cells as P-0734 (FIGS. 11C & 11D and Table 13). Yet, the cell-based binding assay was able to distinguish P-1193, which contains no framework reversion mutation, from other compounds, as shown in FIGS. 11C & 11D and Table 13. The extent of the decrease, though, was less pronounced than what was observed in the blocking assay. The data further corroborate our earlier observation that the mechanistic-based PD1/PD-L1 blocking assay is more sensitive than binding assays in identifying subtle activity differences.

TABLE 13
Binding strength of exemplary optimized
PD1 blocking antibodies to PD1+ cells

PD1+ cell binding EC₅₀ (pM)

| PD1 Ab | % Positive cells | MFI |
|---|---|---|
| P-0734 | 23 | 97 |
| P-1193 | 70 | 259 |
| P-1198 | 19 | 87 |
| P-1199 | 16 | 120 |
| P-1201 | 33 | 159 |

In summary, the optimized PD1 blocking antibodies, P-1194, P-1201 and P-1238, built on a VH framework (IGHV3-23) that is of substantially lower sequence homology but superior biophysical properties, are able to fully or nearly fully retain antibody's functional activity and display improved similarity score to the closest human germline sequence (IGHV3-23). The mutation details and similarity scores for each antibody are summarized in Table 14.

TABLE 14
CDR germlining substitutions, FR reversion mutations, and similarity scores
to the closest human germline sequences of exemplary optimized PD1 antibodies

| | | No. of FR | VH FR | VL Similarity | VH Similarity |
|---|---|---|---|---|---|
| | CDR germling | germlining | reversion | Score to | Score to |
| Ab ID | substitutions | substitutions | mutations | IGKV3D-11 | IGHV3-23 |
| P-0734 | N/A | N/A | N/A | 85.1% | 51.0% |
| P-1194 | ^{L}K27Q, ^{L}L54R, | 30 | ^{H}I69L, ^{H}R71T, | 87.2% | 83.7% |
| | ^{H}F59Y, ^{H}N60A, | | ^{H}N73S | | |
| | ^{H}E61D, ^{H}N65G | | | | |
| P-1201 | ^{L}K27Q, ^{L}L54R, | 30 | ^{H}I69L, ^{H}R71T, | 87.2% | 82.7% |
| | ^{H}N60A, ^{H}E61D, | | ^{H}N73S | | |
| | ^{H}N65G | | | | |
| P-1238 | ^{L}K27Q, ^{L}L54R, | 28 | ^{H}V48M, | 87.2% | 81.6% |
| | ^{H}F59Y, ^{H}N60A, | | ^{H}S49G, ^{H}169L, | | |
| | ^{H}E61D, ^{H}N65G | | ^{H}R71T, ^{H}N73S | | |

### Example 6

### Germlining Substitutions Let to Decreased Hydrophobicity of the Optimized PD1 Blocking Antibodies

Among the 23 FDA and EMA approved therapeutic mAbs, pembrolizumab was the most hydrophobic one and consequently had the highest tendency to aggregate (Goyon et al., J. Chromatogr. B 1065-1066: 35-43, 2017). Consistent with the experimentally-determined apparent hydrophobic interaction chromatography (HIC) retention factors (k), the SSH2.0 hydrophobicity prediction tool (http://i.uestc.edu.cn/SSH2/; Zhou et al., Front. Genet. 13: 842127, 2022) indicated that both variable chains of pembrolizumab carry a significant risk of hydrophobic interaction. The probabilities of hydrophobic interaction for its VH and VL are 0.97 and 0.61, respectively. An antibody is predicted to have a high risk of hydrophobic interaction if the probability is 0.5 or more (with 1 being the maximum possible value).

While the focus of the germlining substitutions was to enhance the degree of “humanness” in the antibody sequence, the process also resulted in a significant reduction in the probabilities of hydrophobic interaction for multiple optimized antibody sequences. Table 15 provides a summary of the predicted probabilities of hydrophobic interaction for the variable domains of the exemplary optimized PD1 blocking antibody, as estimated by SSH2.0.

TABLE 15
Summary of exemplary antibodies with improved similarity
scores and reduced probabilities for hydrophobicity
interaction while retaining PD1 blocking activity

| | SEQ ID NOS | | Similarity score to | | Probability of | | PD1 |
|---|---|---|---|---|---|---|---|
| | Light | Heavy | human germline | | hydrophobic interaction | | blocking |
| PD1 Ab | chain | chain | VL | VH | VL | VH | activity |
| P-0734 | 42 | 43 | 85.1% | 79.6% | 0.607 | 0.971 | Reference |
| P-1174 | 44 | 45 | 87.2% | 83.7% | 0.131 | 0.848 | Fully retain |
| P-1271 | 44 | 49 | 87.2% | 88.8% | 0.131 | 0.185 | Fully retain |
| P-1194 | 44 | 46 | 87.2% | 83.7% | 0.131 | 0.794 | Almost fully |
| | | | | | | | retain |
| P-1201 | 44 | 47 | 87.2% | 82.7% | 0.131 | 0.794 | Almost fully |
| | | | | | | | retain |
| P-1238 | 44 | 48 | 87.2% | 81.6% | 0.131 | 0.740 | Fully retain |

As shown in Table 15, the two light chain CDR germlining substitutions, ^{L}K27Q and ^{L}L54R, markedly decreased the hydrophobicity probability of VL from 0.607 for P-0734 to 0.131. These two amino acid changes were applied to the VL in all the optimized PD1 blocking antibodies listed in Table 15. The CDR germline substitutions in the heavy chain only resulted in a marginal reduction in hydrophobicity, with hydrophobicity probabilities altering from 0.971 for (P-0734) to 0.848 for (P-1174) and to approximately 0.8 for antibodies with their VH based on VH-3 family frameworks. However, when germlining substitutions (^{H}V9A, ^{H}T76S, ^{H}K82aS, ^{H}Q83R, ^{H}F84S, ^{H}T108L) were implemented in the VH framework of P-1174, the resulting construct, P-1271, had a hydrophobicity probability of 0.185, much lower than that of P-1174.

Hydrophobic patches on an antibody's surface are often implicated as one of the main contributions to its propensity to aggregate. Furthermore, these hydrophobic patches can cause high viscosity. Consequently, the exemplary PD1 blocking antibodies, which have significantly diminished hydrophobic potentials are expected to exhibit improved biophysical properties. PD1-targeted IL-2 immunocytokine and VitoKine fusions constructed using these optimized PD1 blocking antibodies are also projected to have enhanced developability profiles.

### Example 7

### Identifying the Optimal IL-2RαSushi Variant as IL-2 VitoKine's Concealing Moiety Domain

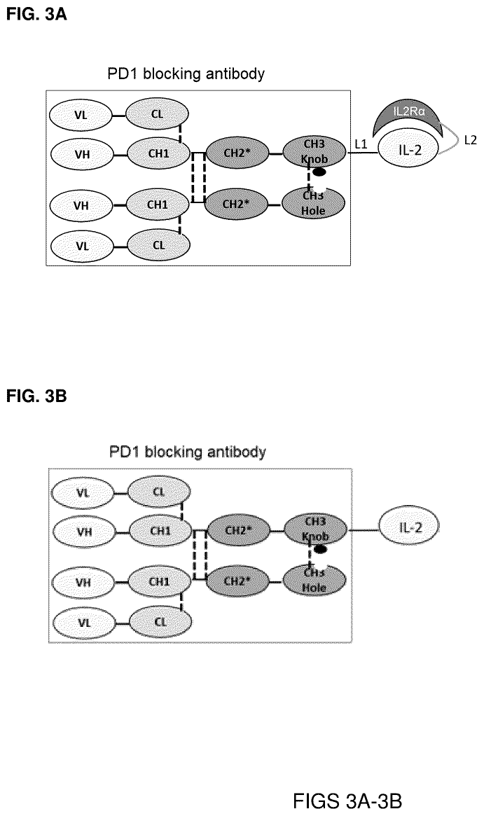

Representative PD1 Ab IL-2 VitoKine construct is illustrated in FIG. 3. A monomeric IL-2 or IL-2 variant as the active moiety domain (D2) is fused between a PD1 antibody (D1) and an IL-2RαSushi domain as the concealing moiety domain (D3). Linker 2 (L2) connecting IL-2 and IL-2Rα is protease cleavable. IL-2 within VitoKine constructs will remain inert until it is activated locally by proteases that are exclusively present or upregulated at tumor sites or within tumor microenvironment (TME). After the L2 linker is cleaved, the concealing a-subunit ideally dissociates away, as illustrated in FIG. 2. Therefore, it is desirable to identify an IL-2Rα variant with weakened binding to IL-2 to ensure that the concealing a-subunit (D3) can readily diffuse away following proteolysis, but still effectively conceals IL-2 moiety domain's activity before the linker is cleaved.

IL-2RαSushi variants were designed to weaken binding to IL-2 by incorporating mutations at residues interacting with IL-2. As listed in Table 16, exemplary IL-2RαSushi variants, P-0751, P-0752, and P-0753 contain the Y43A, L42G, and R36A mutations, respectively. They were expressed as monomeric Fc fusion proteins by fusing to a knob Fc chain of a knob-into-hole heterodimeric Fc chain pair (SEQ ID NOs: 187 and 188). P-0757 is an Fc fusion of the monomeric wild-type IL-2RαSushi. The binding capacity of these three IL-2RαSushi variants to IL-2 was evaluated using an ELISA.

Briefly, IL-2RαSushi variant Fc fusion proteins was coated onto the wells of Nunc Maxisorp 96-well microplates at 1 g/well. After overnight incubation at 4 °C and blocking with 1% BSA, serial dilutions of P-0689, a monomeric wild-type IL-2 equivalent (containing activity-neutral C125I mutation; SEQ ID NO: 117) Fc fusion, were added to each well at 100 L/well. Following a one-hour incubation at room temperature, 100 L/well of biotin anti-IL-2 antibody clone B33-2 (BD biosciences) were added and incubated at room temperature for 1 hour. Subsequently, 100 L/well of Avidin-HRP (BioLegend) at 1:5000 dilution was added. After 60-min incubation and washing, TMB substrate (ThermoFisher) at 100 μL/well was added. Plates were sealed and incubated at room temperature in the dark. Reaction was stopped by adding 2N sulfuric acid (Ricca Chemical). Absorbance was determined at 450 nm and curves were fitted using GraphPad Prism software.

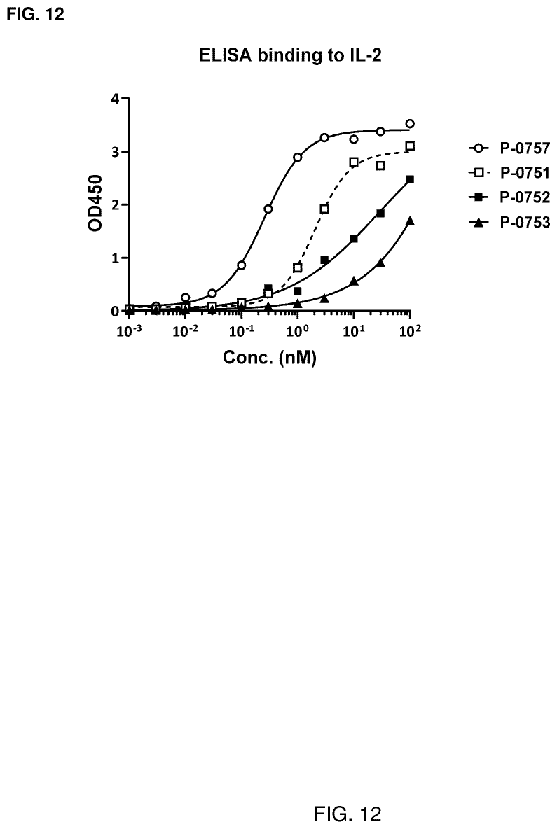

As summarized in Table 16 and illustrated in FIG. 12, the amino acid substitutions Y43A, L42G, and R36A each affected the interaction with IL-2. The Y43A change resulted in a modest reduction in IL-2 binding (8.1-fold), the R36A substitution led to a significant 346-fold drop in binding EC₅₀, while the L42G alteration caused an intermediate, or 35-fold, reduction in its binding to IL-2.

TABLE 16
Impact of IL-2Rα amino acid changes on the binding to IL-2

| Fusion | IL-2RαSushi | IL-2RαSushi | | EC₅₀ fold change vs |
|---|---|---|---|---|
| protein | substitution | SEQ IND NO: | EC₅₀ (nM) | w/t IL-2Rα |
| P-0757 | Wild type | 182 | 0.26 | 1.0 |
| P-0751 | Y43A | 183 | 2.1 | 8.1 |
| P-0752 | L42G | 184 | 9.2 | 35 |
| P-0753 | R36A | 185 | ~50 | ~200 |

The aforementioned three IL-2RαSushi variants, along with their wild-type counterpart, were used as the concealing moiety domain to construct four Fc IL-2 VitoKine molecules. Each of these molecules includes a monomeric IL-2 C125I variant (equivalent to wild-type; SEQ ID NO: 117) as the active moiety domain (D2) and a 15-amino acid MMP2/9-cleavable L2 linker (SEQ ID NO: 84) connecting IL-2 and IL-2RαSushi (D3). Heterodimeric Fc chains (SEQ ID NOS: 187 and 188) served as the D1 domain. The concealing efficiency of these variants were subsequently assessed by evaluating their potency in inducing Ki67 expression, a marker for cell proliferation, in CD8+ T and NK cells via a human PBMC assay. P-0704, an IL-2 P65R variant (SEQ ID NO: 118) Fc fusion maintaining its wild-type IL-2 potency for the dimeric IL-2Rβγ receptor, was included as the fully active IL-2 control for this set of Fc IL-2 VitoKines.

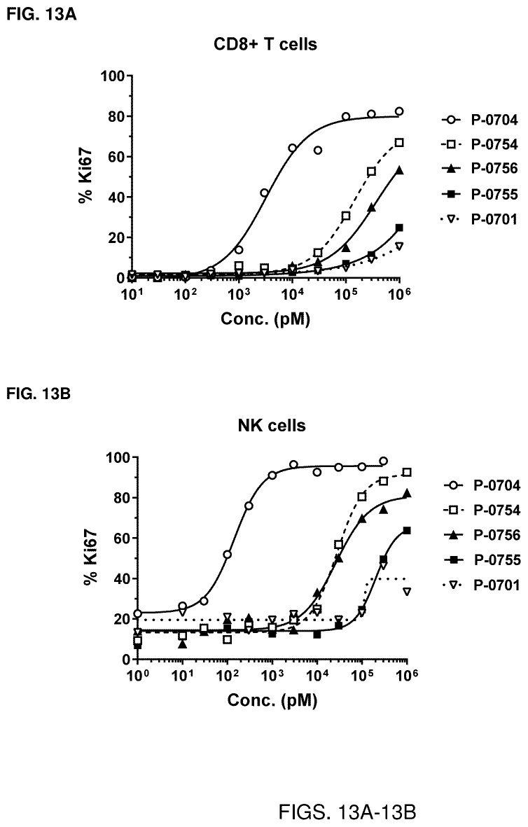

Briefly, human PBMCs were isolated by Ficoll-Hypaque centrifugation from the buffy coat purchased from Blood Oklahoma Institute. Purified human PBMCs were treated with serial dilutions of testing compounds and incubated at 37 °C for 5 days. On the fifth day, cells were washed once with FACS buffer (1% FBS/PBS) and first stained with Fc-blocker (BioLegend) and surface marker antibodies, including anti-human CD56-FITC and anti-human CD8-APC (BioLegend) at a dilution of 1:50. After 30-minute incubation and wash, cell pellets were fully resuspended by 200 L/well of 1× fixation & permeabilization working solution (Invitrogen) and incubated for 30 minutes at room temperature in the dark. After centrifugation, 200 L of 1× permeabilization buffer (Invitrogen) were added to each well for another wash. Cell pellets were resuspended in permeabilization buffer containing anti-human Ki67-PE (BD Life Sciences) at a 1:25 dilution. After a further 30-minute incubation at room temperature, cells were collected, washed, and resuspended in FACS buffer, and analyzed by flow cytometry. Data were expressed as the percentage of Ki67 positive cells within gated population. The dose-responsive Ki67 proliferation details are illustrated in FIGS. 13A and 13B. Additionally, data specific to CD8+ T cells is summarized in Table 17.

TABLE 17
Activity comparison of various IL-2 VitoKine constructs

| | | | | CD8+ T cells | |
|---|---|---|---|---|---|
| Fusion | | IL-2RαSushi | D3 SEQ | | Fold change over P-0704 |
| Protein | Format | (D3) mutation | ID NO: | EC50 (nM) | (concealing efficiency) |
| P-0704 | Fc fusion | N/A | N/A | 3.2 | 1.0 |
| P-0701 | Fc VitoKine | Wild-type | 182 | ~3000 | ~1000 |
| P-0754 | Fc VitoKine | Y43A | 183 | 144 | 45 |
| P-0756 | Fc VitoKine | R36A | 184 | 367 | 115 |
| P-0755 | Fc VitoKine | L42G | 185 | ~3000 | ~1000 |

FIG. 13 illustrates that P-0701, which has a wild-type IL-2RαSushi as the concealing moiety domain (D3), showed a significant 3-log reduction in inducing the proliferation of CD8+ T and NK cells compared to P-0704, its fully active IL-2 Fc fusion counterpart. We hypothesized integrating an IL-2-binding disrupting mutation into the D3 domain might weaken D3's concealing capability, thereby lessening VitoKine's activity inertness. It was also projected that the extent of this reduction would align with the level of diminution in the binding strength between IL-2 and IL-2RαSushi variants.

In FIG. 13A and Table 17, Fc VitoKines P-0754 and P-0756, which contain Y43A and R36A in IL-2RαSushi as the D3 domain, displayed weakened concealing capability, compared to P-0701. This leads to higher VitoKine intrinsic basal in stimulating CD8+ T cell proliferation, and the trend was consistent in NK cells as seen in FIG. 13B. The decrease in concealing efficiency, however, did not consistently correlate with the magnitude of binding strength reduction. For instance, the Y43A mutation had a minor effect on binding, showing only an 8.1-fold decrease, while the R36A mutation caused a substantial ~200-fold drop in binding. Moreover, the L42G variant, despite having a 35-fold weaker binding to IL-2, maintained its concealing effect nearly identical to its wild-type counterpart, as seen in the activity inertness of its corresponding VitoKine, P-0755 (FIGS. 13A and 13B). Although it was unexpected based on previous knowledge, multiple experiments confirmed that changes in binding strength, resulting from mutations in the IL-2RαSushi, didn't consistently correlate with alterations in its concealing capability. This inconsistency might be attributed to the unique spatial interactions in the VitoKine format.

Consequently, IL-2RαSushi L42G variant is selected as the preferred concealing moiety domain (D3) for IL-2 VitoKines due to its retained concealing capability to maintain corresponding VitoKine's activity inertness and its potential to readily diffusing away upon in vivo proteolysis to achieve full activity, given its weakened binding to IL-2. Meanwhile, R36A or Y43A can be used as the concealing moiety domain when one aims to tune the IL-2 VitoKine's intrinsic basal activity, optimizing the balance between desired antitumor efficacy and potential systematic toxicity. Additionally, other IL-2RαSushi variants with varying degrees of reduced binding to IL-2, e.g., K38E, can be used as the D3 domain of IL-2 VitoKines, following the same rationale.

### Example 8

### Substituting Amino Acids at P65 Surprisingly Resulted in a Diverse Impact on the Binding to IL-2Rα

Preferential expansion of regulatory T cells (Tregs) by IL-2 due to the high and constitutive expression of IL-2Rα on Tregs represents an undesirable effect of IL-2 for cancer immunotherapy. IL-2 variants designed to weaken or abrogate binding to IL-2Rα will reduce their responsiveness to Tregs. IL-2 variants that no longer bind to IL-2Rα is expected not to preferentially activate Tregs, but only activate Tregs at concentrations when CD8+ T and NK cells are also activated.

The IL-2 molecule's P65 residue engages Van der Waals interactions with key residues on the IL-2Rα interface, notably R36 and L42. However, it doesn't form salt bridges or hydrogen bonds with IL-2Rα (Xinquan Wang, et. al, Science (2005), 310: 1159-1163). Given this, one might assume that changes to P65 would only slightly alter its interaction with the IL-2Rα subunit and likely only cause a minimal effect on binding. Yet, the actual effects of P65 modifications on its interaction with IL-2Rα were surprisingly diverse, ranging from fully maintaining or even improving binding, to weakening or totally abolishing it.

A panel of IL-2 variants with different P65 substitutions were fused to Fc via a flexible GS based linker (SEQ ID NO: 103), in either dimeric or monomeric formats. All these variants contain an activity-neutral C125I mutation, intended to enhance their developability. Their binding affinity to IL-2Rα (CD25) was then assessed using an ELISA. Briefly, IL-2Rα-ECD was coated onto the wells at 0.1 g/well. After overnight incubation at 4 °C and blocking, serial dilutions of IL-2 Fc fusion proteins were added to each well at 100 L/well. Following a one-hour incubation at room temperature, 100 L/well of goat anti-human IgG Fc-HRP (1:5000 diluted in diluent) were added to each well and incubated at room temperature for 1 hour. The plate was developed at room temperature in the dark following the addition of 100 L TMB substrate for 10 minutes, and 100 L/well of stop solution was added. Absorbance was determined at 450 nm and curves were fitted using Prism software (GraphPad).

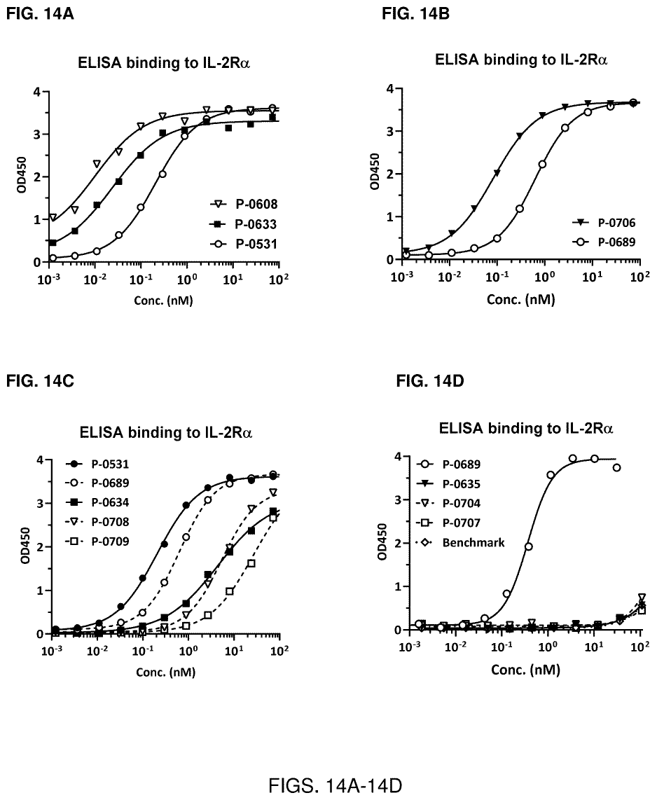

The ELISA binding curves are illustrated in FIG. 14. Additionally, the ELISA binding EC₅₀ values for the IL-2 variants, normalized to that of the wild-type (either P-0531 or P-0689 according to each construct's valency), are detailed in Table 18.

TABLE 18
Binding of IL-2 variants with P65 mutations to IL-2Rα as assessed by ELISA

| Fusion | Fc SEQ | IL-2 SEQ | IL-2 | | Binding EC₅₀ vs. P- |
|---|---|---|---|---|---|
| protein | ID NO(s): | ID NO: | substitutions | IL-2 valency | 0531/ P-0689 |
| P-0531 | 186 | 117 | C125I | Dimeric | 1.0 |
| P-0689 | 187 + 188 | 117 | C125I | Monomeric | 1.0 |
| P-0608 | 186 | 123 | P65G/C125I | Dimeric | 0.055 |
| P-0633 | 186 | 124 | P65E/C125I | Dimeric | 0.1 |
| P-0706 | 187 + 188 | 125 | P65A/C125I | Monomeric | 0.14 |
| P-0708 | 187 + 188 | 120 | P65N/C125I | Monomeric | 8.6 |
| P-0634 | 186 | 122 | P65H/C125I | Dimeric | 23 |
| P-0709 | 187 + 188 | 121 | P65Q/C125I | Monomeric | 43 |
| P-0635 | 186 | 118 | P65R/C125I | Dimeric | >500 |
| P-0704 | 187 + 188 | 118 | P65R/C125I | Monomeric | >500 |
| P-0707 | 187 + 188 | 119 | P65K/C125I | Monovalent | >500 |

As illustrated in FIGS. 14A and 14B, P65G mutation in P-0608, P65E mutation in P-0633, and P65A mutation in P-0706 did not seem to impair the interaction with the IL-2Rα subunit. Rather, these mutations enhanced the binding affinity to IL-2Rα by 18-fold, 10-fold, and 10-fold, respectively, when compared to their respective wild-type IL-2 controls.

In another set, IL-2 variant Fc fusions, namely P-0634, P-0708, and P-0709, carried P65 alterations leading to different levels of disturbances in the bond with the IL-2Rα subunit. As showcased in FIG. 14C and detailed in Table 10, the P65N mutation in P-0708 resulted in a moderate 8.6-fold decline in IL-2Rα binding. On the other hand, the P65H (P-0634) and P65Q (P-0709) alterations had a more pronounced effect, causing reductions in binding by 23-fold and 43-fold, respectively.

Another group of P65 substitutions, specifically P65R and P65K, appeared to engender drastic disruption in the IL-2 and IL-R2Rα interaction, completely eliminating the binding of P-0635, P-0704, and P-0707 to IL-2Rα. Here, P-0635 and P-0704 are the dimeric and monomeric versions of the IL-2 P65R variant Fc fusions, and P-0707 harbors the P65K amino acid change. FIG. 14D reveals that these three IL-2 mutein Fc fusions had barely any detectable binding signal, even at IL-2Rα concentrations as high as 100 nM. This is on par with the benchmark molecule, which carries three CD25-disrupting mutations F42A/Y45A/L72G known to eliminate binding, as documented in Christian Klein et al., Oncolmmunology (2017), 6: 3, e1277306.

In summary, alterations to the P65 residue led to a diverse range of effects on IL-2Rα binding, including increasing, maintaining, reducing, or completely abrogating the binding of the resultant IL-2 variants to IL-2Rα. Such a broad spectrum of outcomes, arising from modifications to what appears to be a non-critical amino acid, could not be resulted from the predictions of a structure-based mutagenesis approach. The complete loss of IL-2Rα binding was unforeseen and not predicted by the prior art. This is especially surprising given that P65 mutations only modified a small portion of the Van der Waals interaction surface.

It is expected that the change in IL-2Rα binding strength correlates with IL-2 potency in activating Treg cells. To validate this, IL-2 variant Fc fusion proteins with either enhanced binding (P-0608), reduced binding (P-0634 and P-0709) or abolished binding (P-0635 and P-0704) to IL-2Rα were examined for their ability to differentially stimulate STAT5 phosphorylation in CD4+ Treg cells. STAT5 is known to be involved in the downstream signaling cascade upon IL-2 binding to the transmembrane IL-2 receptors. Wild-type IL-2 fusion P-0531 and the benchmark molecule were included for comparison.

The phosphorylation of STAT5 in lymphocyte subpopulations was measured in fresh human PBMCs using the transcription factor FOXP3 to identify the Treg population in FACS analysis. Specifically, purified PBMCs were deprived of serum in MACS buffer (Miltenyi Biotech) at 4 °C for 1 hour, and subsequently treated with serial dilutions of test compounds for 30 min at 37 °C. Cells were then fixed, permeabilized, stained with specific antibodies, and further analyzed by flow cytometry following a similar procedure detailed in Example 7. The staining was achieved using a mixture of anti-CD25-PE, anti-FOXP3-APC, anti-pSTAT5-FITC, and anti-CD4-PerCP-Cy5.5 antibodies (purchased from BioLegend or BD Life Sciences). The flow cytometry data was gated into CD4+/Foxp3+/CD25high group for the Treg cell subsets. Data are expressed as the percentage of pStat5 positive cells in the gated population.

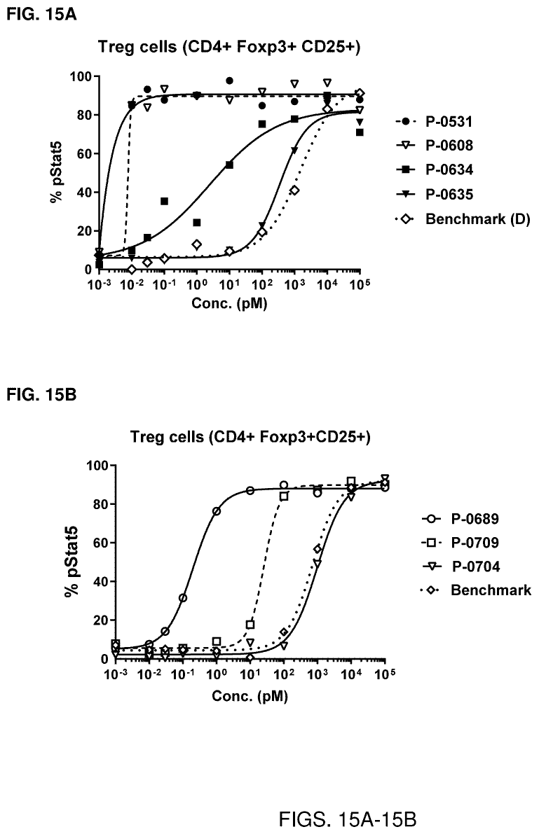

As illustrated in FIG. 15, there is a clear correlation between the IL-2Rα binding strength and the potency in stimulating STAT5 phosphorylation in CD4+ Treg cells. The compounds in FIG. 15A all feature bivalent IL-2 variants while those in FIG. 15B all have monomeric IL-2 variants. P-0608, which has enhanced IL-2Rα binding, displayed appreciably higher potency compared to P-0531. P-0626 (FIG. 15A) and P-0709 (FIG. 15B), in line with its diminished IL-2Rα binding strength, showed reduced pSTAT5 potency compared to P-0531/P-0689. However, its remaining, albeit reduced, binding to IL-2Rα ensured that Tregs were activated more effectively than both P-0635/P-0689 and the benchmark molecules (valency-matched), which completely lost IL-2Rα binding. Similarly, the total loss of IL-2Rα binding led to a significant shift in Treg potency, about 5 logs to the right. This remaining Treg signaling was resulted from the activation of IL-Rpy found on Treg cells.

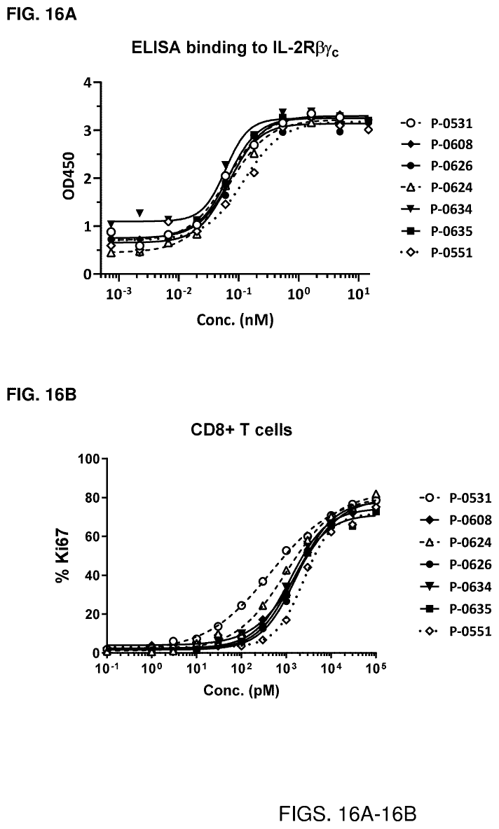

Further, the exemplary IL-2 variant Fc fusions, which included mutations rendering enhanced, reduced, or abolished binding to IL-2Rα, all displayed un-altered binding to IL-2Rβγ (FIG. 16A). They also showed nearly equal potency in inducing Ki-67 expression in CD8+ T cells (FIG. 16B). The data underscores the fact that the IL-2 mutations at IL-2Rα interface, regardless of their effect on IL-2Rα binding, do not alter the interaction with IL-2Rβγ.

### Example 9

### Identifying IL-2 Variants with Optimal IL-2Rα Binding as VitoKines' Active Moiety Domain

The active moiety domain (D2) of IL-2 VitoKine constructs were selected from a panel of IL-2 variants with varying levels of binding strength to IL-2Rα identified in Example 8. Incorporating IL-2 variants with reduced or eliminated IL-2Rα binding in VitoKines can decrease the reactivity to Tregs following proteolytic activation near the tumor. However, it is essential to achieve a balance between the degree of weakened IL-2Rα binding and the efficiency of concealment, given that binding between D2 and D3 is believed to be necessary for the concealing capability of VitoKines.

The four exemplary IL-2 VitoKines, namely P-0800, P-0830, P-0831, and P-0802, all contain an anti-mouse PD1 antibody P-0722 (SEQ ID NOS: 52, 189 and 190) as the D1 domain, IL-2RαSushi L42G variant (SEQ ID NO: 184) as the concealing moiety domain (D3), a non-cleavable linker (SEQ ID NO: 103) as the L1 linker, and an MMP-2/9 cleavable linker (SEQ ID NO: 84) as the L2 linker. As detailed in Table 19, the active domain (D2) comprises P65R mutation in P-0800, P65N mutation in P-0830, and P65Q mutation in P-0831. P-0802 has the IL-2 wild-type equivalent as the D2 domain.

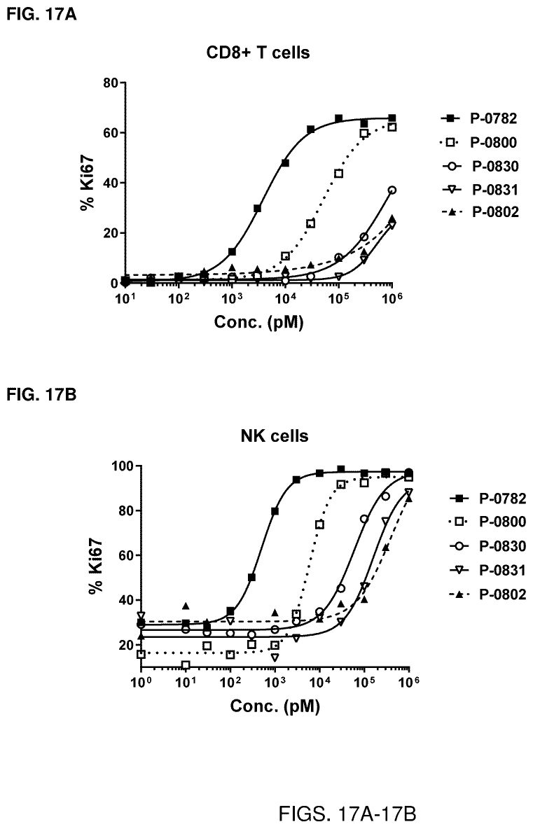

The exemplary VitoKines were assessed for their ability to induce Ki67 expression on CD8+ T cells (FIG. 17A) and NK cells (FIG. 17B) using fresh human PBMCs. P-0782, a non-VitoKine immunocytokine counterpart of P-0800 containing a monomeric IL-2 P65R variant, was included as a fully active IL-2 reference. The EC₅₀ values of these compounds in terms of stimulating Ki67 expression in NK cells, along with their fold changes in comparison to P-0782, are summarized in Table 19.

The data revealed that when the D2 domain is the wild-type IL-2, the D3 domain of the VitoKine construct (P-0802) renders a roughly 3-log reduction in activity, suggesting a strong concealment capability of the D3 domain. In contrast, for P-0800, which incorporates an IL-2 variant (P65R) with abolished IL-2Rα binding, there is only a 10- to 20-fold decrease in activity, suggesting a significantly weakening of the D3's concealment capability due to the absence of binding between the D2 and D3 domains. Interestingly, when using IL-2 variants like P65N and P65Q, which have intermediately reduced IL-2Rα binding, in P-0830 and P-0831, the D3 domain renders concealing efficiency that is either comparable to, or only marginally less than that it facilitates wild-type IL-2.

TABLE 19
Activity comparison of IL-2 VitoKines containing IL-2
variants with varying IL- 2Rα binding strength

| | | | % Ki67 expression in NK cells | |
|---|---|---|---|---|
| PD1 Ab-IL-2 | IL-2 (D2) | D2 SEQ | | Fold change |
| VitoKine | mutation | ID NO: | EC₅₀ (nM) | over P-0782 |
| P-0800 | P65R/C125I | 118 | 5.87 | 12 |
| P-0830 | P65N/C125I | 120 | 59.3 | 118 |
| P-0831 | P65Q/C125I | 121 | 159 | 317 |
| P-0802 | C125I | 117 | 397 | 792 |

It was postulated that a binding affinity threshold between the D2 and D3 domains, combined with an ideal spatial configuration of the binding interface, is pivotal in determining the concealing efficiency of the IL-2 VitoKines. Given that IL-2 P65Q variant demonstrates significantly reduced binding strength to IL-2Rα (as depicted in FIG. 14C and Table 18) and yet is still efficiently concealed by IL-2RαSushi L42G to remain inert as VitoKine, IL-2 P65Q variant is chosen as the preferred D2 domain for IL-2 VitoKine designs. It is expected the D3 domain will be readily diffused away following protease cleavage due to the diminished binding between the D2 and D3 domains. It is also anticipated that once the bioactivity is fully restored upon proteolytic activation, this variant will have a significantly reduced ability to in stimulate Treg cells compared to the wild-type, as illustrated in FIG. 15B. Nevertheless, other IL-2 variants with reduced IL-2Rα binding, such as P65H and P65N, can also be considered to achieve the right balance between desired antitumor efficacy and minimizing potential systematic toxicity.

On top of the P65 mutations to achieve a balance between the weakened binding and effective concealment by the IL-2RαSushi, additional mutations altering IL-2's binding affinity to IL-2Rβγ can be incorporated into VitoKines' D2 domain. These mutations modulate IL-2's overall responses in cells that mainly express β and γ receptor subunits, such as CD8+ T and NK cells. Through this strategy, the intrinsic basal activity of the VitoKines can be fine-tuned as well as their activity post-proteolytic activation.

### Example 10

### IL-2 Variants with IL-2Rβγ-Disrupting Substitutions for Overall Potency Attenuation

The choice of mutations disrupting IL-2Rβ and the common γ chain (γc) was informed by inspecting the IL-2/IL-2R co-crystal structure (PDB code 2B51). Substituting energy hot spot residues that directly interact with IL-2Rβ, such as D20 and N88, could result in significantly reduced activity, rendering poor potency. Consequently, substitutions were introduced at non-critical residues at the IL-2/IL-2Rβ interface, such as L19. The L19 residue only makes van der Waals interaction with IL-2Rβ, and the resulting mutants are expected to only tweak, rather than significantly diminish, IL-2's functional activity. Further, replacing L19 with a non-aliphatic residue eliminates a proposed ‘¹⁹LDL’ motif, which may contribute to the vascular toxicity of IL-2 (Baluna R, Rizo et. al., Proc Natl Acad Sci 1999; 96:3957-62).

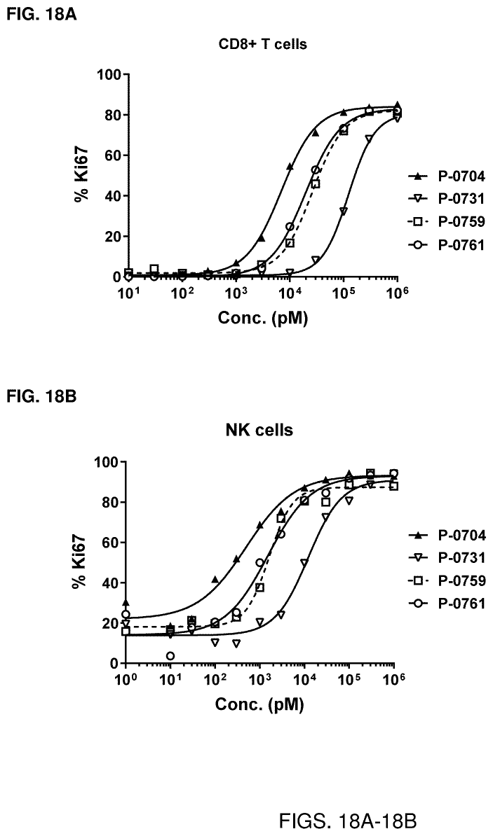

Exemplary IL-2Rβ-disrupting mutations, L19H, L19Q, L19Y, were introduced into IL-2 with the mutational context of P65R and C125I in P-0704 to construct monomeric IL-2 Fc fusions. The P65R mutation led to a total loss of IL-2Rα binding and the C125I modification was made for developability purpose, and neither of these changes impacted IL-2's functional activity for IL-2Rβγ. The resulting fusion proteins, namely P-0731, P-0759, and P-0761, were assessed for their potency in stimulating Ki67 expression on human CD8+ T cells and NK cells by flow cytometry. The results are depicted in FIGS. 18A and 18B and detailed in Table 20A. When compared to P-0704, all variants displayed a decrease in their ability to promote proliferation in human CD8+ T cells and NK cells. Specifically, P-0759 (L19Q) and P-0761 (L19Y) exhibited a modest 3-fold decline in potency while L19H mutation in P-0731 led to a more profound 18-25-fold drop in potency. Potency reduction can also be achieved by incorporating other L19 mutations, such as L19D, L19R, and L19S.

TABLE 20A
Exemplary Fc fusions of IL-2 variants containing
L19 mutations and their ex vivo activity

| | | | % Ki67 in CD8+ T cells | | % Ki67 in NK cells | |
|---|---|---|---|---|---|---|
| IL-2 Fc | Substitution | IL-2 SEQ | EC₅₀ | Fold | EC₅₀ | Fold |
| fusion | for L19 | ID NO: | (nM) | change | (nM) | change |
| P-0704 | / | 118 | 6.97 | 1.0 | 0.496 | 1.0 |
| P-0761 | Y | 153 | 19.7 | 2.8 | 1.54 | 3.1 |
| P-0759 | Q | 152 | 26.5 | 3.8 | 1.62 | 3.3 |
| P-0731 | H | 151 | 124 | 18 | 12.2 | 25 |

Likewise, amino acid substitutions at Q126, a residue that is integral to the γc interaction, were made to weaken IL-2 interaction with γc. All these mutations were also introduced into IL-2 with the mutational context of P65R and C125I. Fc fusions of monomeric IL-2 variants containing Q126 mutations were listed in Table 20B.

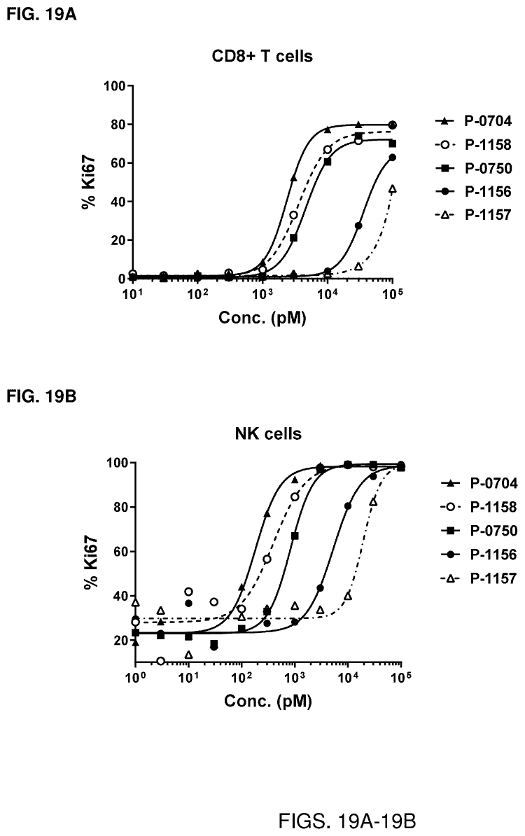

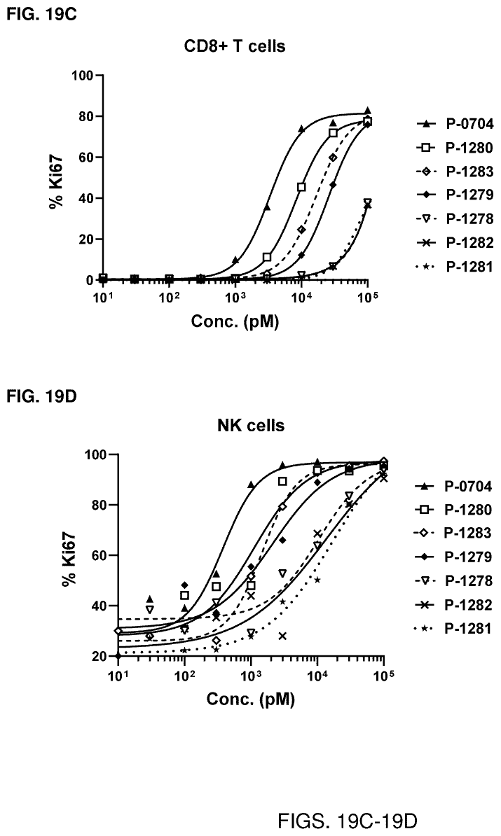

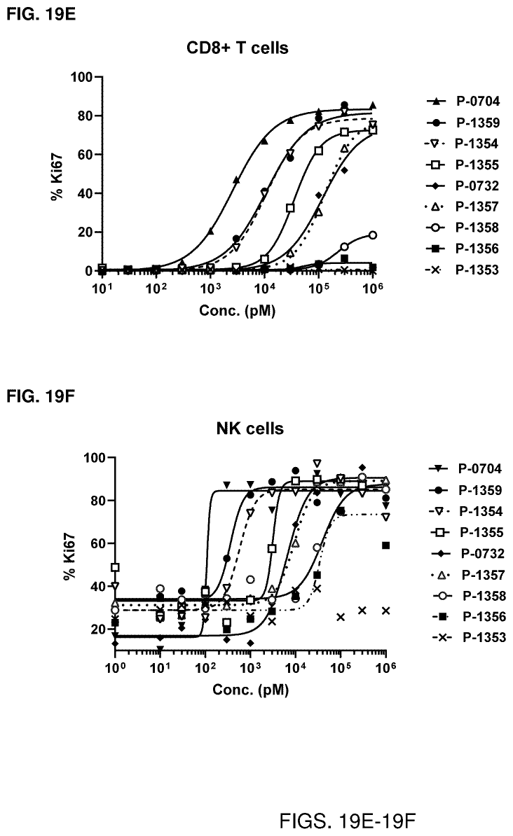

Increases in the Ki67 expression of human CD8+ T cells and NK cells in response to these IL-2 variants, compared to P-0704, were depicted in FIGS. 19A to 19F, and further summarized in Table 20B. IL-2 Q126 Mutations had varying degrees of impact on CD8+T and NK cell proliferation. For CD8+ T cells (FIGS. 19A, 19C, and 19E), a minor 1.5-5-fold decrease was shown by Q126N, Q126H, Q126M, Q126F, Q126W, and Q126Y; a moderate 5-20-fold reduction was observed for Q126R, Q126G, Q126S; a significant 20-50-fold decline was noted for mutations including Q126A, Q126V, Q126E, Q126E, Q126L, and Q126T; a more drastic drop (>50-fold) was seen for Q126P and Q1261; and Q126D completely abolished the activity. A comparable trend in potency changes due to 0126 mutations was observed in NK cells (FIGS. 19B, 19D, and 19F).

TABLE 20B
Fc fusions of IL-2 variants containing Q126 mutations and their ex vivo activity

| | | | % Ki67 in CD8+ T cells | | % Ki67 in NK cells | |
|---|---|---|---|---|---|---|
| IL-2 Fc | Substitution | IL-2 SEQ | EC₅₀ | Fold | EC₅₀ | Fold |
| fusions | for Q126 | ID NO: | (nM) | change | (nM) | change |
| P-0704 | / | 118 | 2.78 ± 0.50 | 1.0 | 0.25 ± 0.11 | 1.0 |
| P-1158 | N | 165 | 3.60 | 1.5 | 0.39 | 2.2 |
| P-0750 | H | 160 | 4.60 | 1.9 | 0.83 | 4.6 |
| P-1280 | M | 164 | 8.4 | 2.5 | 1.46 | 3.8 |
| P-1354 | F | 158 | 10.6 | 3.6 | 0.57 | 3.2 |
| P-1359 | W | 171 | 10.8 | 4.1 | 0.36 | 2.0 |
| P-1283 | Y | 172 | 16.7 | 5.0 | 1.47 | 3.9 |
| P-1279 | R | 167 | 26.0 | 7.8 | 2.12 | 5.6 |
| P-1355 | G | 159 | 35.3 | 13 | 3.13 | 17 |
| P-1156 | S | 168 | 35.8 | 15 | 7.32 | 52 |
| P-1281 | A | 155 | ~100 | ~30 | 15.3 | 40 |
| P-1282 | V | 170 | ~100 | ~30 | 9.79 | 26 |
| P-1278 | K | 162 | ~100 | ~30 | 11.2 | 29 |
| P-0732 | E | 157 | 118 | 45 | 6.48 | 85 |
| P-1357 | L | 163 | 128 | 49 | 9.36 | 52 |
| P-1157 | T | 169 | ~100 | ~50 | 19.4 | 108 |
| P-1358 | P | 166 | 229 | 88 | 39.2 | 218 |
| P-1356 | I | 161 | weak | >100 | 34.3 | 191 |
| P-1353 | D | 156 | N/D | N/A | N/D | N/A |

*Fold change was compared to P-0704's EC₅₀ value in each individual experiment.

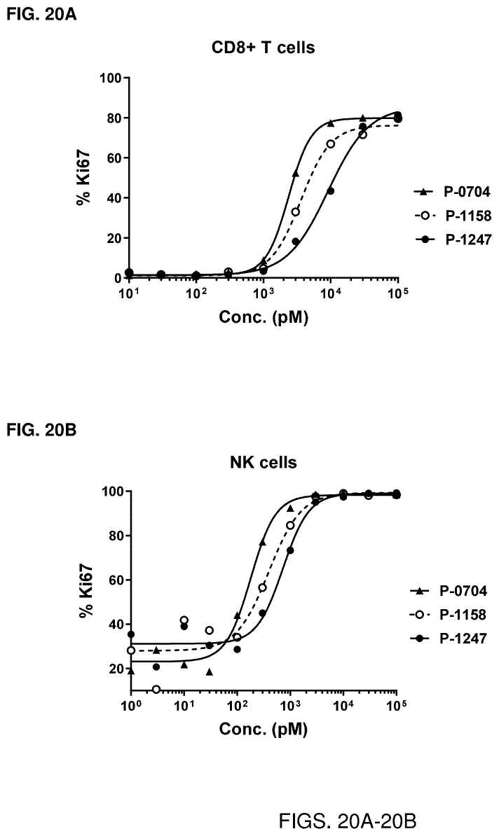

IL-2 potency can be further fine-tuned by combining IL-2R(3 and γc-disrupting mutations, as exemplified by P-1247 (IL-2 domain SEQ ID NO: 173) in comparison to P-1158 and P-0704. In addition to the P65R and C125I mutations in P-0704, P-1158 contains the Q126N mutation and P-1247 comprises the L19Y and Q126N mutations. As shown FIG. 20A, the incorporation of the L19Y mutation in P-1247 resulted in an additive 2.6-fold decrease in potency compared to P-1158 (9.2 nM vs 3.6 nM) and a combined 4-fold potency reduction compared to P-0704 (9.2 nM vs 2.3 nM) for the stimulation of Ki67 expression in human CD8+ T cells. A similar trend was seen with NK cells (FIG. 20B). As will be appreciated by those in the art, combining diverse mutations at position L19 and Q126 can lead to various degrees of activity modulation, and is within the spirit and scope of the invention.

In summary, in addition to using IL-2Rα-disrupting substitution in IL-2 to limit the undesirable expansion of immunosuppressive Tregs, integrating IL-2Rβγ-disrupting substitutions offers a way to attenuate the overall potency for optimal activity. By introducing specific mutations at either L19 or Q126, varying degree of potency can be achieved. The desired potency of IL-2 can be meticulously fine-tuned through the combination of mutations at the L19 and Q126 positions. A reduced potency help avoid excessive pathway activation of the pathway and minimize unwanted target sink. As a result, the strategy can potentially reduce toxicity associated with IL-2 therapy and improve pharmacokinetics and pharmacodynamics. Incorporating IL-2 with reduced potency in VitoKines helps fine-tune their intrinsic basal activity as well as their activity post-proteolytic activation.

### Example 11

### Constructing PD1 Ab-IL-2 VitoKines Using Optimized PD1 Blocking Antibodies and the Preferred IL-2 and IL-2RαSushi Domains

Antibodies that block PD1 and thus bypass the immunosuppressive effects in the tumor microenvironment may potentiate IL-2 responses and further enhance immunity against tumors. The PD1 antibodies used to construct PD1 Ab-IL-2 VitoKines as the D1 domain were selected from the optimized human PD1 blocking antibodies comprising light chain sequences set forth in SEQ ID NO: 44 and heavy chain sequences set forth in SEQ ID NOS: 45-49. These optimized PD1 blocking antibodies have a high affinity for human PD1 protein and demonstrate equal or comparable potency as pembrolizumab in blocking PD1. They also possess a higher sequence similarity score to their closest human germline sequence, resulting in an improved degree of humanness compared to pembrolizumab. Furthermore, they are predicted to have lower hydrophobicity, which in turn is likely to lower their aggregation propensity than pembrolizumab. PD1-targeted IL-2 VitoKines constructed using these optimized PD1 blocking antibodies are also projected to have enhanced developability profiles.

Table 21 lists the exemplary PD1 Ab-IL-2 VitoKines, with their structure depicted in FIG. 3A. All the exemplary VitoKines comprise IL-2 P65Q variant with or without mutations to modulate activity towards IL-2Rβγ as the active moiety domain (D2), IL-2RαSushi L42G variant as the concealing moiety domain (D3), and a cleavable L2 linker ySEQ ID NO: 84) connecting D2 and D3 domains. Nevertheless, other IL-2RαSushi variants, e.g., R36A, can be used as the concealing moiety domain when it is desirable to adjust the IL-2 VitoKine's intrinsic basal activity. Additionally, the L1 linker connecting PD1 Ab and IL-2 can be cleavable as well. The compositions of the cleavable linker(s) can be further optimized by using the various sequences set forth in SEQ ID NOS: 78-94, to better suit different disease indications and/or stages.

TABLE 21A
Exemplary human PD1 Ab-IL-2 VitoKines

| PD1 | Component | | |
|---|---|---|---|
| Ab-IL-2 | human | Mutations | |
| VitoKine | PD1 Ab | in IL-2 (D2) | VitoKine Seq ID NOS: |
| P-1197 | P-1174 | P65Q/C125I | 198 + 199 + 44 |
| P-1239 | P-1238 | P65Q/C125I | 200 + 201 + 44 |
| P-1272 | P-1271 | P65Q/C125I | 202 + 203 + 44 |
| P-0872 | P-0795 | P65Q/C125I | 194 + 195 + 50 |
| P-1120 | P-0734 | P65Q/C125I | 196 + 197 + 42 |
| P-1394 | P-1271 | L19Q/P65Q/C125I | 209 + 203 + 44 |
| P-1386 | P-1271 | P65Q/C125I/Q126N | 204 + 203 + 44 |
| P-1387 | P-1271 | P65Q/C125I/Q126H | 205 + 203 + 44 |
| P-1388 | P-1271 | P65Q/C125I/Q126M | 206 + 203 + 44 |
| P-1389 | P-1271 | P65Q/C125I/Q126F | 207 + 203 + 44 |
| P-1390 | P-1271 | P65Q/C125I/Q126R | 208 + 203 + 44 |

All genes were codon optimized for expression in mammalian cells, which were synthesized and subcloned into the recipient mammalian expression vector via the service of GenScript. The VitoKine constructs were produced by co-transfecting Expi293 cells (ThermoFisher) with the mammalian expression vectors following manufacturer's instructions. Protein purification and characterization were conducted following the same procedures detailed in Example 2.

It was found that the PD1 Ab-IL-2 VitoKines comprising optimized antibody sequences, including P-1197, P-1239, and P-1272, expressed at a substantially higher level than P-1120 that contains the Reference Antibody, P-0734. Under the identical transient expression conditions using the same batch of Expi293 cells, P-1197, P-1239, and P-1272 expressed with a titer of 137-150 mg/L compared to 60 mg/L for P-1120. The data suggested that the PD1 blocking antibodies with optimized sequences eliminating potential sequence liabilities may lead to improved developability properties of the corresponding VitoKine constructs.

Because these optimized PD1 antibodies did not react with mouse PD1, surrogate mouse PD1-Ab-IL-2 VitoKines and additional controls were produced analogously. These were used for in vivo studies, particularly for pharmacokinetics (PK)/pharmacodynamics (PD) and tumor experiments in immunocompetent mice. Table 21 B provides detailed information about these molecules. Every VitoKine construct listed in this table incorporates the same D3 domain (SEQ ID NO: 184). With the exception of P-871, these VitoKines contain the mouse PD1 antibody P-0722 (SEQ IND NO: 189, 190, and 52) as the D1 domain. P-0871, a non-targeting VitoKine counterpart of P-0831, contains the germline antibody P-1260 (SEQ ID NOS: 192, 193, and 194) as the D1 domain. P-0877 is a non-cleavable VitoKine, and P-0838, which lacks the L2 linker and D3 and has its structure illustrated in FIG. 3B, serves as the non-VitoKine immunocytokine counterpart to P-0831.

TABLE 21B
Exemplary surrogate mouse PD1 Ab-IL-2 VitoKines and control molecules

| Ab-IL-2 | Component Ab (D1) | | IL-2 SEQ | L1 linker | L2 linker |
|---|---|---|---|---|---|
| VitoKine | SEQ ID NOS: | Mutations in IL-2 (D2) | ID NO: | SEQ ID NO | SEQ ID NO: |
| P-0831 | 189 + 190 + 52 | P65Q/C125I | 121 | 103 | 84 |
| P-1386 | 189 + 190 + 52 | P65Q/C125I/Q126N | 176 | 103 | 84 |
| P-1387 | 189 + 190 + 52 | P65Q/C125I/Q126H | 177 | 103 | 84 |
| P-1388 | 189 + 190 + 52 | P65Q/C125I/Q126M | 178 | 103 | 84 |
| P-1389 | 189 + 190 + 52 | P65Q/C125I/Q126F | 179 | 103 | 84 |
| P-1390 | 189 + 190 + 52 | P65Q/C125I/Q126R | 180 | 103 | 84 |
| P-1393 | 189 + 190 + 52 | L19Q/P65Q/C125I | 154 | 103 | 84 |
| P-0877 | 189 + 190 + 52 | P65Q/C125I | 121 | 103 | 115 |
| P-0871 | 191 + 192 + 193 | P65Q/C125I | 121 | 103 | 84 |
| P-1345 | 189 + 190 + 52 | P65Q/C125I | 121 | 84 | 115 |

While P-0831 is the primary subject for the in vivo studies of the following examples, other VitoKines containing IL-2 variant with additional mutations to target IL-2Rβγ (such as the ones listed in Table 21 B), are predicted to achieve similar anti-tumor effectiveness when the dosage is properly adjusted. Since the intrinsic basal activity of IL-2 VitoKine correlates directly with the activity of its active domain (D2), a weakened D2 activity and the proportionally modulated VitoKine's basal activity enables the administration of higher doses without adverse effects. This helps fully support PD1 antibody's function of reversing T-cell anergy or exhaustion, thereby potentially enhancing the synergistic effects with IL-2 immunotherapy and broadening the therapeutic window.

### Example 12

### Ex Vivo Activity and In Vitro Proteolytic Activation of PD1 Ab-IL-2 VitoKines

It is important that the PD1 antibody retains its binding and functional activities when incorporated into PD1 Ab-IL-2 VitoKines. PD1 antibodies of superior target-binding and PD1 blocking function can enhance the specificity and selectivity of TIL-targeting, and further synergize with IL-2 anticancer immune response by efficiently reversing T cell anergy and exhaustion.

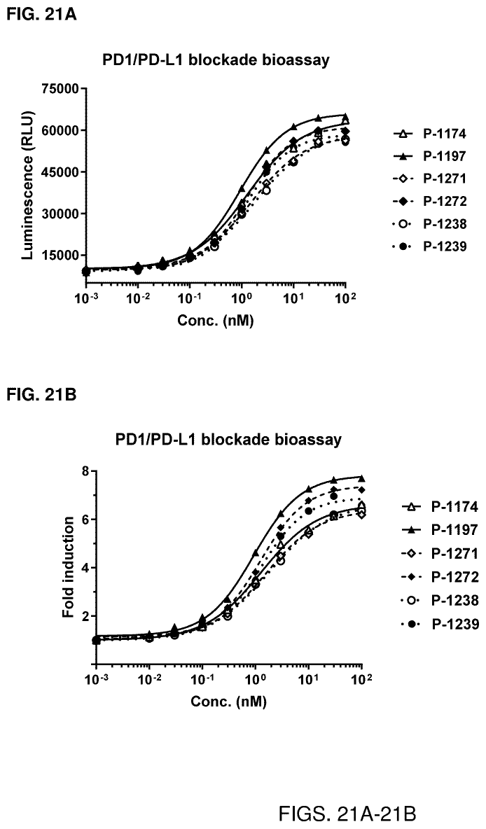

In a comparative analysis using a luciferase reporter assay, the PD1 inhibition capabilities of exemplary VitoKines P-1197, P-1239, and P-1272 were set against their respective PD1 blocking antibodies, P-1174, P-1238, and P-1271. As illustrated in FIGS. 21A and 21B, each of the three antibodies, when incorporated into their corresponding VitoKine constructs, not only maintained their blocking potency but also slightly improved it. For instance, PD1 antibodies P-1174, P-1238, and P-1271 blocked the PD1/PD-L1 interaction with EC₅₀ values of 1.29 nM, 1.82 nM, and 1.53 nM, respectively. On the other hand, their corresponding VitoKines, P-1197, P-1239, and P-1272, displayed a subtle enhancement of 1.5-2-times in the blocking efficiency, with EC₅₀ values of 0.92 nM, 1.27 nM, and 1.20 nM, respectively. Additionally, in the VitoKine format, there was an 11-17% increase in both Eₘₐₓ and fold induction.

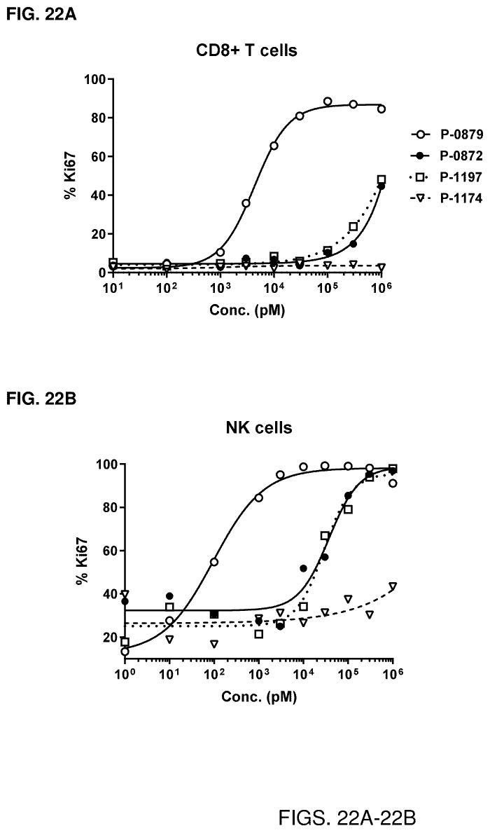

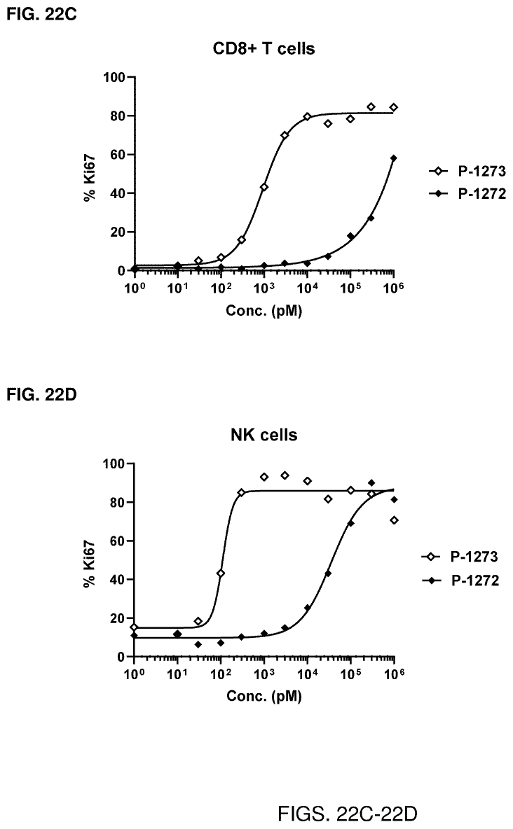

FIG. 22 further confirmed that the activity of IL-2 remains effectively concealed by the IL-2RαSushi domain, irrespective of the specific PD1 antibody compositions. Exemplary PD1 Ab IL-2 VitoKines, P-1197, P-1272, and P-0872, which differ only based on their distinct PD1 blocking antibodies (detailed in Table 21A), all show an approximate 300-fold decrease in their ability to induce the proliferation of CD8+ T and NK cells when compared P-0879 (FIGS. 22A and 22B) or P-1273 (FIGS. 22C and 22D). For context, P-0879 and P-1273 are respectively the non-VitoKine immunocytokine counterparts of P-0872 of P-1272, lacking the concealing D3 domain. The EC₅₀ values can be found in Table 22. For CD8+ T cells, the low potency of VitoKines prevented the curve fitting, resulting in only approximate EC₅₀ values. P-1174 is the PD1 antibody component of P-1197 and was included as a negative control.

TABLE 22
EC₅₀ values of exemplary PD1 Ab-IL-2 VitoKines Compared
to their Non-VitoKine Immunocytokine Counterparts

| | | | | % Ki67 in |
|---|---|---|---|---|
| Fusion | PD1 | | Ki67 in CD8+ T | NK Cells |
| Protein | Ab | Format | Cells EC₅₀ (nM) | EC₅₀ (nM) |
| P-0879 | P-0795 | Non-VitoKine | 3.60 | 0.096 |
| P-0872 | P-0795 | VitoKine | ~800 | 37.0 |
| P-1197 | P-1174 | VitoKine | ~700 | 28.9 |
| P-1273 | P-1271 | Non-VitoKine | 0.933 | 0.111 |
| P-1272 | P-1271 | VitoKine | ~400 | 35.4 |

P-1272 was further assessed for in vitro proteolytic activation. Consistent with other exemplary PD1 Ab-IL-2 VitoKines in the present invention, P1272 contains a single MMP-2/9 cleavable L2 linker (SEQ ID NO: 84). In the procedure, 3.3 g of latent MMP-2 (BioLegend) was first activated by APMA (Millipore Sigma) according to the manufacturer's instruction, which was then buffer exchanged and added to 120 g of P-1272 in 0.4 ml of the manufacture recommended assay buffer (100 mM Tris, 20 mM CaCl₂, 300 mM NaCl, 0.1% (w/v) Brij 35, pH 7.5). After a 3-hour incubation at 37 °C, the treated sample was then purified using protein A resin (MabSelect SuRe; Cytiva) in a bind-elute mode. The eluted sample was analyzed in a reduced SDS-PAGE gel, and its biological function was assessed in an ex vivo functional assay.

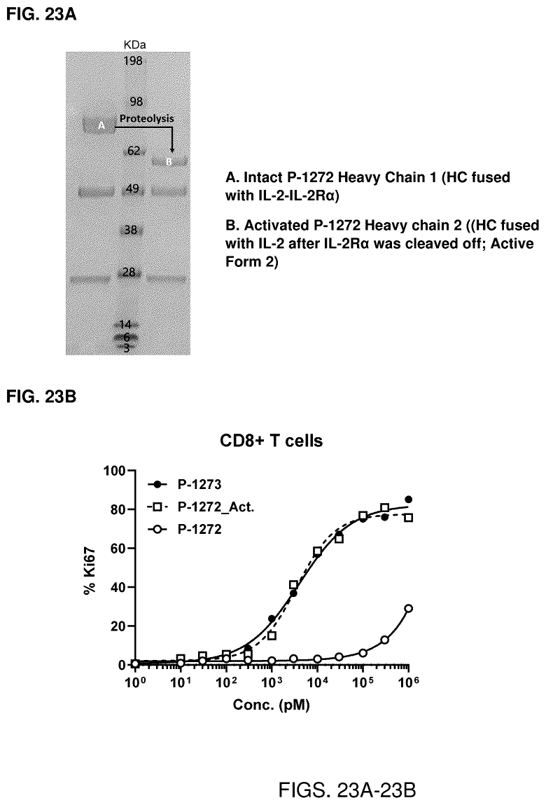

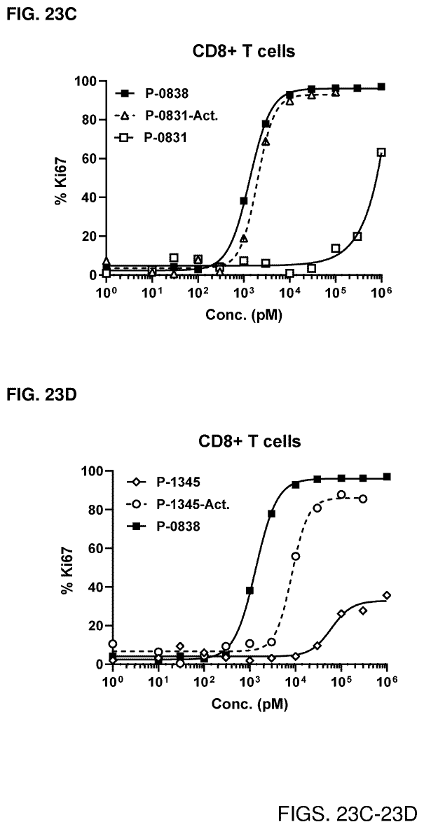

FIG. 23A illustrates that the concealing moiety domain of P-1272 was effectively and fully cleaved, resulting in P-1272-Activ., which corresponds to Active Form 2 depicted in FIG. 2. Efficient in vitro proteolysis led to a full restoration of the activity of IL-2, exemplified by the indistinguishable activity of P-1272-Activ. and P-1273 in inducing a dose-dependent expression of Ki67 in CD8+ T cell of fresh human PBMCs (FIG. 23B). Comparable findings were observed with other PD1 Ab-IL-2 VitoKines, as exemplified by P-0831, and are depicted in FIG. 23C.

Moreover, P-1345, a distinct form of PD1 Ab-IL-2 VitoKine, differs from other VitoKines in that it includes a cleavable L1 linker (SEQ ID NO: 84) and a non-cleavable L2 linker (SEQ ID NO: 115). P-1345 was similarly activated through in vitro protease cleavage, and its sole activated form, referred to as Active Form 1 in FIG. 2, was isolated. This form was then evaluated for its potency in inducing Ki67 expression in CD8+ T cells of human PBMCs. As demonstrated in FIG. 23D, proteolytic activation led to an approximate 50-fold increase in activity compared to its intact VitoKine form. However, the activation did not fully restore the activity of the IL-2 domain, showing it to be 6 times less active than its non-VitoKine counterpart, P-0838, with the respective EC₅₀ values of 8.29 nM and 1.34 nM.

These results imply that employing a cleavable L2 linker is more advantageous than a cleavable L1 linker. This is because Active Form 2, derived from the cleavage of the L2 linker, is a fully functional IL-2 domain fused to the PD1 Ab. This form can activate IL-2R signaling in PD1-expressing T cells near the disease site, enhancing both pathways and synergizing the anticancer immune response, while reducing systemic toxicity. On the other hand, Active Form 1 exhibits reduced potency, a shorter half-life, and lacks TIL-targeting capabilities.

These observations also suggest that the concealing domain by itself is insufficient for effective concealment of the active domain, resulting in a modest concealing efficiency of approximately 6-folde. For efficient concealment of IL-2 activity, the structure must be in the form of the VitoKine platform disclosed herein as well as in WO2019246392 and WO2021119516 by the current inventors, which involves the coupling of both the targeting domain (D1) and the concealing domain (D3).

### Example 13

### Prolonged In Vivo Half-Life of PD1-Ab-IL-2 VitoKine in Non-Tumor Bearing Mice

The VitoKine's IL-2 domain is designed to remain inert until locally activated by proteases upregulated in diseased tissues. As a result, it is expected that the IL-2 VitoKine's binding to IL-2 receptors on cell surface in peripheral and non-diseased tissues will be markedly diminished. This would mitigate potential antigen sink and/or target-mediated deposition, consequently extending the in vivo half-life. A study was carried out to compare the pharmacokinetics of a mouse PD1 Ab IL-2 VitoKine, P-0831, with its non-VitoKine immunocytokine counterpart, P-0838, in non-tumor bearing C57BL/6 mice.

Naïve female C57BL/6 mice, aged seven weeks, were received from Charles River Laboratory. Before starting the study, they were allowed a 7-day acclimation in house. At the start, P-0831 and P-0838, each at a dose of 1 mg/kg, were administered by intravenous injection. Vehicle (PBS) was included as the negative control. Blood samples were withdrawn at 10 min, 2 hours, 6 hours, 24 hours, 48 hours, 72 hours, 120 hours, 168 hours, 240 hours, and 360 hours post-injection by cheek bleeding. Each group consisted of 3 mice, and blood was taken either weekly or every three days, with a maximum frequency of twice per group.

The serum concentrations of the compounds were determined using ELISA assays. Three different ELISA methods were developed for P-0831 to measure: 1) total VitoKine concentration (including both activated and intact forms); 2) concentration of intact VitoKine; and 3) concentration of the activated VitoKine. For all the three methods, maxisorp plates were coated with mouse PD1 protein (R&D systems) overnight at 4 °C. Following this, plates were blocked with Superblock (ThermoFisher). Blood samples at various dilutions were added to the plates and incubated for one hour at room temperature.

For total VitoKine detection, an anti-IL-2 goat polyclonal antibody (R&D Systems) was added, followed by a secondary HRP-conjugated donkey anti-goat IgG (ThermoFisher). To detect intact VitoKine, a polyclonal anti-CD25 antibody (R&D Systems) was used, which was probed using HRP-conjugated streptavidin protein (ThermoFisher). For activated VitoKine detection, a biotinylated monoclonal anti-IL-2 antibody (BD Pharmingen) was applied, paired with HRP-conjugated streptavidin. For P-0838 detection, the same anti-IL-2 goat polyclonal antibody (R&D Systems) for detecting total VitoKine concentration was used followed by the donkey anti-goat IgG-HRP. The resultant signals were developed using the Ultra TMB substrate solution, and values were extrapolated from non-linear regression curve fits in GraphPad Prism.

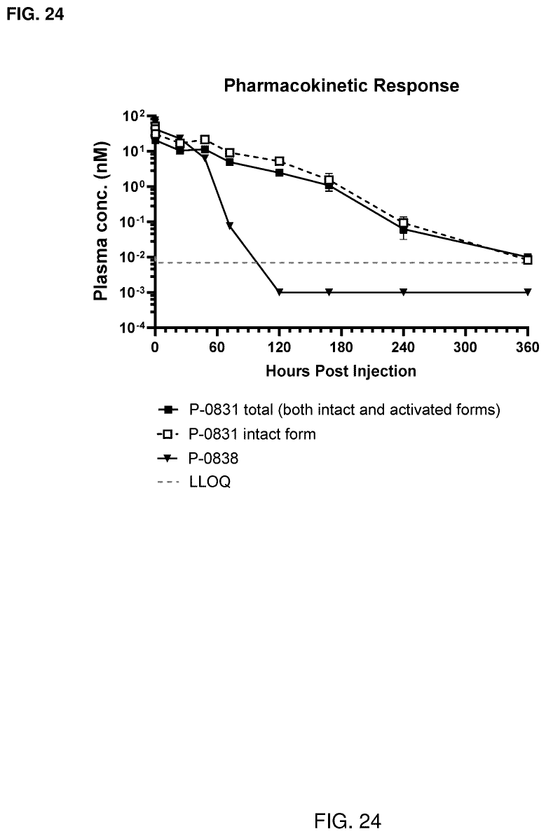

As shown in FIG. 24, the concentration profiles for the intact P-0831 and the total (including both intact and activated forms) P-0831 align closely, suggesting that P-0831 predominately circulates in its intact state. Further, there was no evidence of activated P-0831 detected at any time points following administration, corroborating the notion that P-0831 remains intact in the periphery.

Contrasting with the concentration profiles of P-0831, which remained measurable 360 hours after a 1 mg/kg dosage, P-0838's serum concentration dropped quickly. It became substantially lower by 72 hours and was undetectable by 120 hours post-dosing. The grey dashed horizontal line in FIG. 24 donate the lower limit of quantification (LLOQ) for serum P-0838 levels. For measurements falling below the LLOQ, values were assigned as 10³ nM.

The findings strongly support the notion that the VitoKine format is superior in extending the active domain's in vivo half-life. The strikingly prolonged in vivo half-life of the VitoKines is believed to result from the inertness of the IL-2 domain in the peripheral blood. This inactivity likely diminishes the Interaction with IL-2 receptors on the cell surface in both peripheral and non-diseased tissues, considerably reducing cell activation and expansion, and consequently mitigating potential antigen sink and/or target-mediated deposition.

### Example 14

### Minimized Systemic Pharmacodynamic Effects of PD1-Ab-IL-2 VitoKine in Non-Tumor Bearing Mice

The VitoKine platform aims to mitigate systemic on-target toxicity and widen the therapeutic window for cytokine therapy. This is achieved by rendering the active cytokine inert within the construct, which prevents it from interacting with receptors in peripheral blood or on non-diseased cell surfaces. This design helps limit over-activation of the cytokine pathway and reduces the risk of undesirable “on-target” effects in “off tissue” locations. To evaluate this hypothesis, the mouse PD1 Ab-IL-2 VitoKine P-0831 were administered into non-tumor bearing C57BL/6 mice to assess its systemic effects in comparison to its non-VitoKine immunocytokine counterpart, P-0838, by monitoring the proliferation and expansion of peripheral blood lymphocytes over a given period.

Naïve C57BL/6 mice, aged between 7-9 weeks (n=4/group) were administered via a single intraperitoneal injection of P-0831 at 2 and 10 mg/kg, and P-0838 at 0.3, 1, and, 2 mg/kg. Vehicle (PBS) was included as the negative control. Blood samples were collected into heparin tubes on Days 0, 3, 5, 7, and 10 after dosing for immunophenotyping.

Subsequently, the heparin-treated blood samples were stained with a panel of antibodies targeting common surface immune cell markers. After the lysis of red blood cells using BD Pharmingen lysis buffer, the total number of viable mononuclear blood cells were determined by excluding dead cells with trypan blue. The lysed immune cells were then subjected to a 30-minute fixation and permeabilization at room temperature in the dak using the fixation/permeabilization buffer (eBioscience). After washing, these cells were intracellularly stained with antibodies for the Ki67 proliferation marker. The distinct immune cell subsets were identified, and their absolute counts in circulation were quantified using a flow cytometer (Beckton Dickinson). This was done using commercially available antibodies, namely CD3-APC.Cy7, CD8-Percp-cy5.5, CD335-APC, CD45-AF700, CD4-BV421, CD25-BV510, Foxp3-FITC, Ki67-PE, and granzyme B-BV421. Flow cytometry analysis was performed using FlowJo software and the results were plotted using GraphPad Prism.

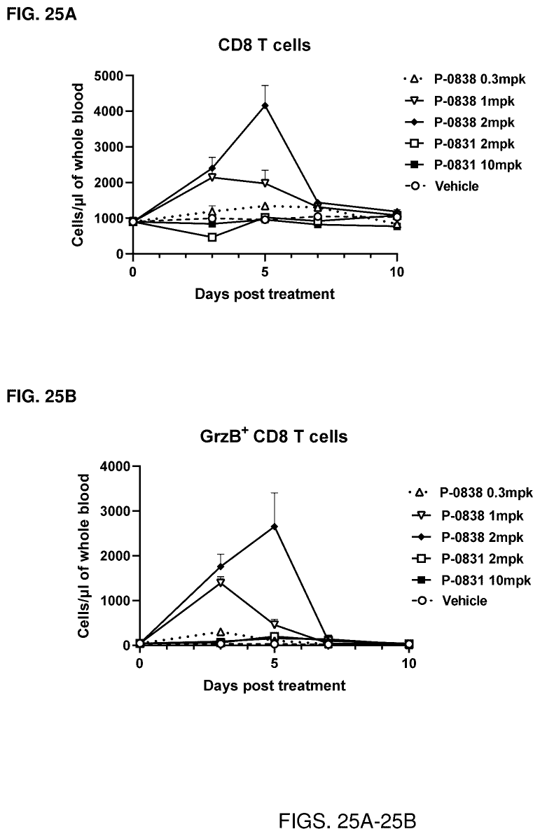

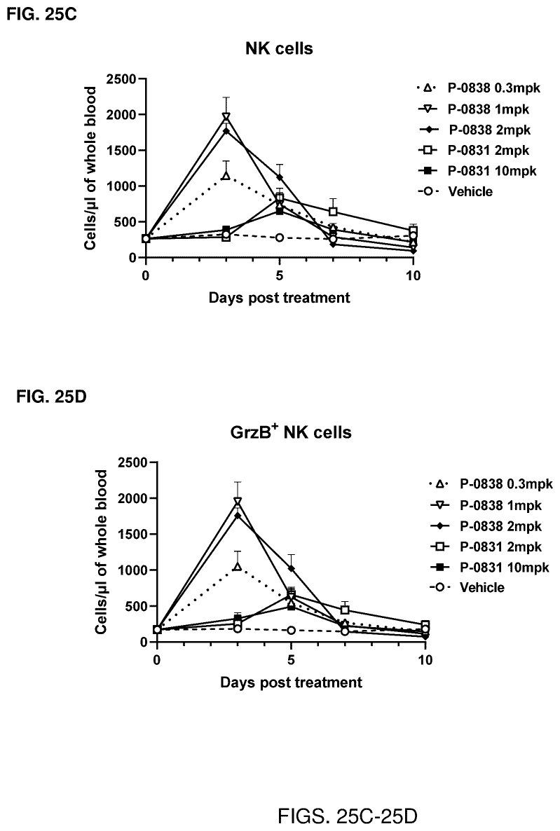

In FIG. 25, the data revealed that P-0838 dramatically expanded peripheral blood CD8+ T cells (FIG. 25A) and granzyme B+CD8+ T cells (FIG. 25B) at doses of 1 and 2 mg/kg, exhibiting a dose-dependent response. Specifically, with a 2 mg/kg dose for P-0838, the CD8+ T cells expanded from a baseline level of 900 cells/μL to 2400 cells/μL (an increase of 2.7-fold) on Day 3. The expansion peaked on Day 5 at 4200 cells/μL (a 4.7-fold increase) before declining to near the baseline level on Day 7. With a 1 mg/kg dose of P-0838, the peak expansion for CD8+ T cell expansion was observed on Day 3, with a 2.3-fold increase, and returned to near the baseline level by Day 7. At a lower dose of 0.3 mg/kg of P-0838, the CD8+ T cell expansion was only slightly noticeable. A similar trend was observed in the expansion of cytotoxic granzyme B+CD8+ T cells (FIG. 25B). In sharp contrast, even at higher doses of 2 mg/kg and 10 mg/kg, VitoKine P-0831 showed no notable expansion of CD8+ T cell or granzyme B+ cell throughout the 10-day period (illustrated in FIGS. 25 A and 25B).

Consistent with the ex vivo assay results, NK cells displayed a higher reactivity to IL-2 treatment than CD8+ T cells. This was evident from the significant NK cell expansion observed with the 0.3 mg/kg dose of P-0838 (FIG. 25C). NK cell expansion showed dose dependence between the 0.3 and 1 mg/kg doses, with no marked differences between the 1 and 2 mg/kg dosages. Peak expansion for NK cells across all three dosages of P-0838 was on Day 3 (FIG. 25C). A similar patten was observed in the expansion of cytotoxic granzyme B+NK cells (FIG. 25D). In contrast, P-0831 treatment led to only slight and delayed increases in the numbers of both NK cells and granzyme B+NK cells, even when administered at a considerably high dose of 10 mg/kg (FIGS. 25C and 25D).

In summary, when contrasted with the active IL-2 fusion molecule P-0838, P-0831 exhibited significantly diminished systemic proliferation and expansion of the specific lymphocytes. This highlights the effectiveness of the VitoKine format in concealing IL-2 activity, thereby preventing unwanted activation of the IL-2 pathway and mitigating the risk of undesirable “on-target” effects in “off tissue”.

### Example 15

### Mitigating Cytokine-Associated Toxicity in Mice with PD1 Ab-IL-2 VitoKines

Cytokine-associated toxicity, also known as cytokine release syndrome (CRS), is one of the main risks associated with cancer immunotherapy. CRS stems from an intense immune response and is often associated with elevated circulating levels of several cytokines, including interleukin-6 and interferon gamma (INFγ). As immune-based therapies increase in potency, the magnitude of immune activation can exceed levels that occurring in more natural settings, potentially escalating CRS to a life-threatening level. Given that the VitoKine platform is designed to limit over-activation of cytokine pathways and considering the exemplary IL-2 VitoKine P-0831's proven capability to minimize systemic activation and expansion of the targeted lymphocyte populations (refer to Example 14), VitoKine hold promise in significantly reducing cytokine-associated toxicity.

To investigate the potential for reducing cytokine-associated toxicity, the mouse PD1 Ab-IL-2 VitoKine P-0831 and its non-VitoKine immunocytokine counterpart P-0838 were administered at varying dosages into non-tumor bearing naïve C57BL/6 mice. Subsequently, the circulating levels of INFγ, one of the key serum inflammatory cytokines, were determined.

Naïve C57BL/6 mice, aged 7-9 weeks old and grouped in sets of three (n=3), were given a single intraperitoneal injection of P-0831 at doses of 1, 3, 6, 10, and 20 mg/kg and P-0838 at doses of 1, 3, and 6 mg/kg. Vehicle (PBS) and the mouse PD1 antibody P-0722 (containing homodimeric Fc with SEQ ID NOS: 52 and 53) were included as the negative controls. Serum samples were collected and isolated from the mice 48 hours after the treatment. The mouse INFγ DuoSet ELISA kit (R&D Systems) was used, following manufacturer's instruction, to determine the serum IFNγ concentration.

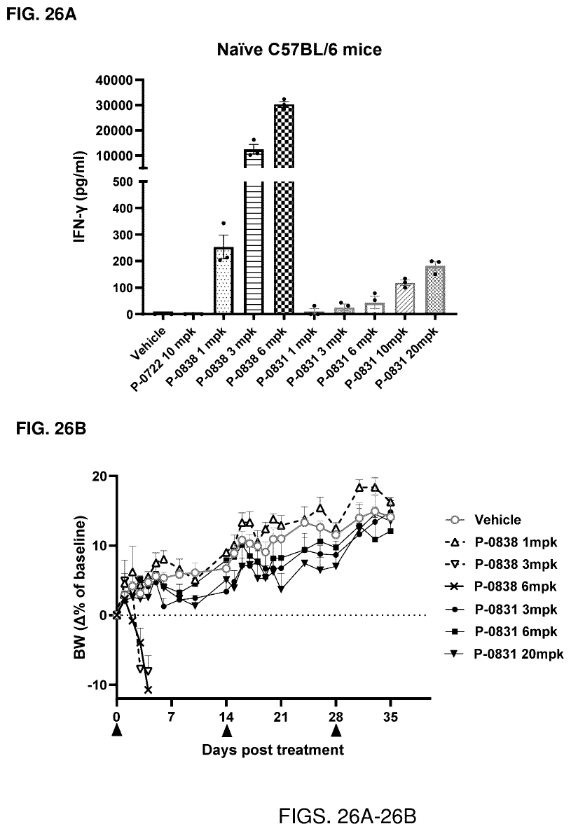

FIG. 26A and the accompanying Table 23 revealed that both P-0831 and P-0838 treatments resulted in dose-dependent increases in the serum IFNγ levels. However, the VitoKine P-0831 exhibited markedly diminished IFNγ serum levels in comparison to P-0838. At a 1 mg/kg dose, the P-0838 treatment resulted in an INFγ concentration of 253 μg/mL, whereas P-0831 only resulted in a mere 10.6 μg/mL. More strikingly, at a 3 mg/kg dose, the serum INFγ for P-0838 soared to 12482 μg/mL, which is 50 times the level seen with the 1 mg/kg dose. In contrast, P-0831, when dosed at 3 mg/kg, only demonstrated a 2.5-fold increase in INFγ levels compared to its 1 mg/kg dose. Surprisingly, even when administered at a 20 mg/kg dose, P-0831 t only led to a modest INFγ level of 182 μg/mL, which is still below the level seen with a 1 mg/kg dose of P-0838. As expected, neither the vehicle nor the antibody treatment induced any discernible release of this inflammatory cytokine, as illustrated in FIG. 26A.

TABLE 23
Serum INFγ concentration 48 hours post-treatment

| | | Serum INFγ Conc. |
|---|---|---|
| Construct ID | Doses | (pg/mL) |
| P-0838 | 1 mg/kg | 253 |
| | 3 mg/kg | 12482 |
| | 6 mg/kg | 30256 |
| P-0831 | 1 mg/kg | 10.6 |
| | 3 mg/kg | 25.1 |
| | 6 mg/kg | 44.0 |
| | 10 mg/kg | 118 |
| | 20 mg/kg | 182 |

The drastic increases in the circulating INFγ levels observed in the P-0838 treatment groups at 3 and 6 mg/kg doses, resulted from high-level systematic immune activation, can lead to severe toxicity. In a concurrently conducted experiment, naïve C57BL/6 mice (7-9 weeks old, n=4/group) were administered with P-0831 at doses of 3, 6, and 20 mg/kg and P-0838 at doses of 1, 3, and 6 mg/kg via intraperitoneal injection. Such cytokine-associated toxic effects were accompanied with significant weight loss (FIG. 26B) and other signs of stress in the mice treated with 3 mg/kg and 6 mg/kg of P-0838. Given the established protocol necessitates the termination of any mice experiencing over 10% body weight, all mice in the P-0838 6 mg/kg treatment group did not survive beyond 4 days, and 3 out of the 4 mice in the 3 mg/kg P-0838 group had to be sacrificed on the 4^{th} day after a single injection. On the contrary, mice treated with P-0831 across all tested doses, even up to 20 mg/kg, remained alive after 3 doses on a Q2W dosing schedule, and exhibited no significant weight loss (FIG. 26B). Thes suggested that the VitoKine platform presents a remarkably lower toxicity profile.

In summary, PD1 Ab IL-2 VitoKines notably mitigated cytokine-associated toxicity in mice, as evidenced by the strikingly reduced circulating levels of inflammatory cytokines, exemplified by INFγ, and minimal changes in body weight even at much higher doses compared to the non-VitoKine immunocytokine counterpart. The PD1 Ab-IL-2 VitoKine platform effectively minimizes off-target toxicity, thereby offering a wider therapeutic window.

Furthermore, the ability of PD1 Ab IL-2 VitoKines to be tolerated at a much-elevated doses provides more flexibility in optimizing dosing regimens. At higher doses, the PD1antibody's function of reversing T-cell anergy or exhaustion can be fully fulfilled since the dose levels are within its effective range, potentially enhancing the synergistic effects with IL-2 immunotherapy. Additionally, incorporating IL-2 with reduced potency, achieved by introducing mutations that disrupt IL-2Rβ or γc interaction, will lead to VitoKines with lower intrinsic basal activity and could potentially further broaden the therapeutic margin.

### Example 16

### Inhibiting the Growth of Established Tumors in Mice by PD1 Ab-IL-2 VitoKines

The anti-tumor efficacy of PD1 Ab IL-2 VitoKine P-0831 was investigated in the syngeneic MC38 murine colon carcinoma model compared to its non-VitoKine immunocytokine counterpart P-0838. In these experiments, female C57BL/6 mice, aged between 7-9 weeks, were implanted subcutaneously in the right flank with 5×10⁵ MC38 colon carcinoma cells. Roughly 2 weeks later, once the tumors had grown to an average volume of ~75 mm³, the mice were randomized into groups of 8 on Study Day 0. On Study Day 1, treatments were given as follows: mouse PD1 antibody P-0722 at 9 mg/kg, P-0831 at varying dosing levels (3 mg/kg, 6 mg/kg, and 9 mg/kg), and P-0838 at 1 mg/kg. These treatments were given intraperitoneally every 10 days (Q10D) for a total of 2 doses. Vehicle (PBS) was used as a control. Both the tumor growth and mouse's body weight were monitored bi-weekly. Tumor volume (TV) was determined using caliper measurement and calculated as: volume=0.5×(width)²×(length). The tumor growth inhibition (TGI, %) was calculated using the following formula: TGI (%)=[1-(TV of the treated group)/(TV of the control group)]×100(%). Based on the established criteria, if a tumor grew to or exceeded 1500 mm³, or it became necrotic, the mouse was euthanized.

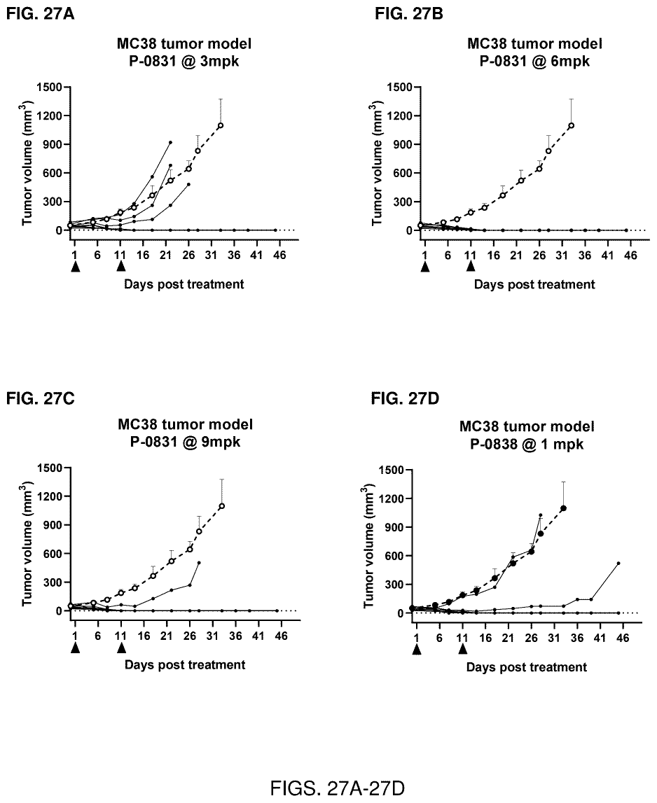

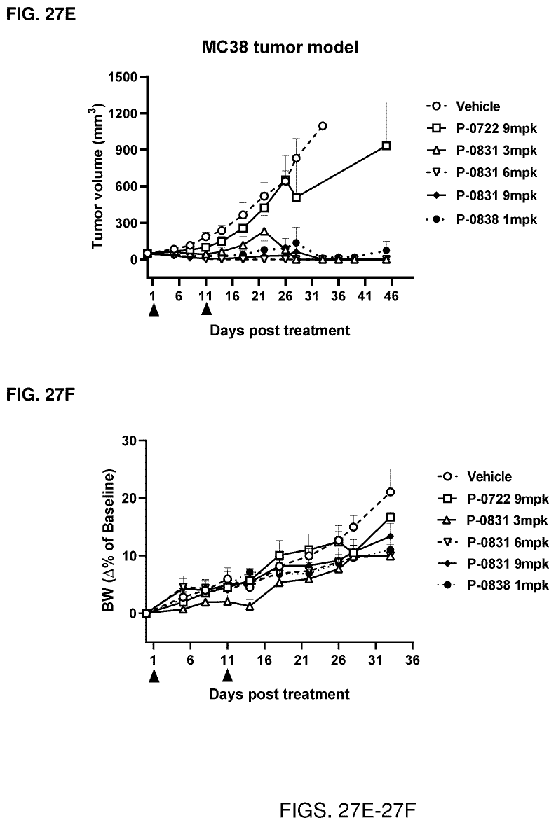

FIGS. 27A-27D illustrate tumor growth curves of individual mice for the four different treatment groups containing the IL-2 moiety. Each line in the graphs represents one mouse, along with the average tumor growth of the vehicle group represented by a dotted line. A small arrow below the X-axis signifies each dose. The treatment with P-0831 at 6 mg/kg demonstrated the most pronounced and sustained effect, with all eight mice from this group completely eradicating tumor growth on Day 45, which is 34 days after the second and final treatment (FIG. 27B). Likewise, in the group treated with P-0831 at 9 mg/kg, 7 out of 8 mice remained tumor-free at the end of study (FIG. 27C). On the other hand, P-0831 administered at 3 mg/kg had slightly lower efficacy, with 5 out 8 mice remaining tumor-free while 3 mice showing tumor growth after an initial period of delayed tumor growth (FIG. 27A). For the mice treated with 1 mg/kg of P-0838, 6 out of 8 mice were tumor-free by the study's conclusion (FIG. 27D).

The mean tumor volume, along with the standard error of the mean (SEM) for each group as a function of time, is further illustrated in FIG. 27E. Mice treated with vehicle rapidly developed large subcutaneous tumors. The PD1 antibody treatment showed limited effectiveness, resulting in a 27% tumor growth inhibition (TGI) when compared to the vehicle group. Conversely, all other treatment groups exhibited high efficacy in inhibiting tumor growth with a 100% TGI on Day 45 after the start of the treatment.

FIG. 27F shows that P-0831 was well tolerated with little or no body weight loss, even at dosages considerably higher than P-0838. It was previously shown that P-0838 was not tolerated at doses of 3 mg/kg or more (as shown in FIG. 26B). The combined in vivo findings indicate that a 6 mg/kg dosage of P-0831 led to a more pronounced and prolonged response. Notably, this anti-tumor efficacy was achieved with considerably lower peripheral lymphocyte proliferation and expansion (as seen in FIG. 25), as well as a notably diminished production of circulating INFγ (FIG. 26) when compared to the effects of a 1 mg/kg dosage of P-0838. This efficacy is partly attributed to the high dose tolerance afforded by the VitoKine format. Consequently, PD1 Ab IL-2 VitoKine platform provides a wider therapeutic window, enabling the antibody component to fully achieve its potential in reversing T-cell anergy and exhaustion.

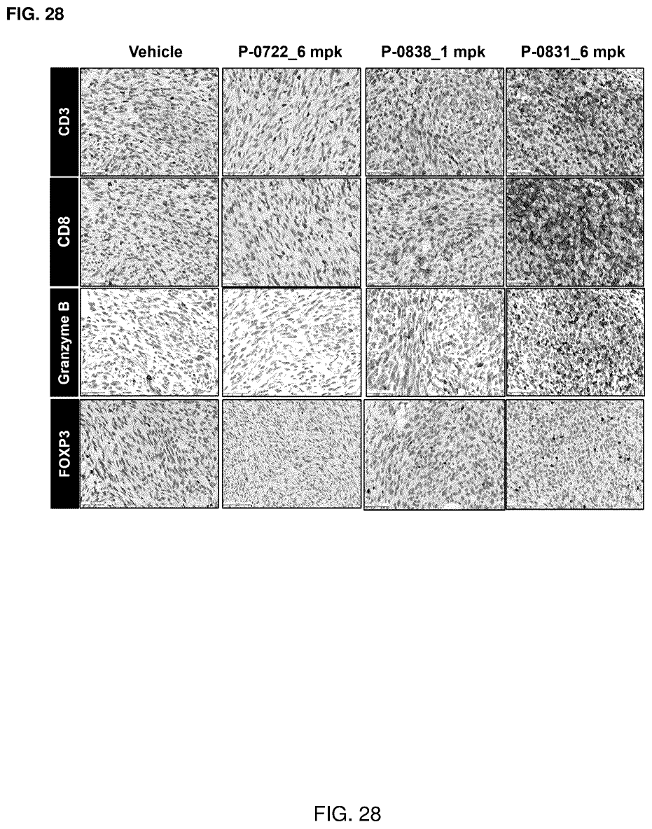

In a parallel study, immunohistochemistry (IHC) was utilized to assess the impact of PD1 Ab-IL-2 VitoKine on tumor tissues obtained five days after treatment. In this setup, MC38 subcutaneous tumors were established similarly. After randomization (5 mice/group), the mice were treated with a single dose of either vehicle, P-0722 (6 mg/kg), P-0831 (6 mg/kg), or P-0838 (1 mg/kg). Five days post-treatment, the mice were euthanized, tumors extracted, and tissue samples prepared. The tissue sections were fixed in 10% formalin, paraffin-embedded, processed, and stained with antibodies following the manufacturer's instructions by HistoWiz to evaluate immune cells in the tumor tissue. Representative IHC images for each group are illustrated in FIG. 28.

FIG. 28 showcases that P-0831 treatment elicited extensive infiltration of CD3+ T and CD8+ T cells into the tumor tissues. Furthermore, the infiltrated CD8+ T cells were characterized by a strong cytotoxic capability, evidenced by intensive granzyme B expression. These observations highlight the amplified number and activity of cytotoxic CD8+ T cells in the tumors of P-0831-treated mice, further validating the anti-tumor efficacy data. In sharp contrast, the mouse PD1 Antibody R00722 at 6 mg/kg induced only minimal tumor-infiltrating lymphocyte (TIL) presence. While P-0838 treatment led to limited T cell infiltration, it did demonstrate high granzyme B expression. Importantly, no treatment notably resulted in the presence of the inhibitory FOXP3+ cells.

Taken together, PD1 Ab IL-2 VitoKine, exemplified by the surrogate molecule P-0831, effectively inhibited tumor growth by promoting extensive infiltration of cytotoxic T cells into the tumor tissues while minimizing the proliferation and expansion of peripheral lymphocytes. Consequently, using the VitoKine format could mitigate issues commonly associated with fully active cytokine, such as excessive stimulation of the immune pathway, undesirable “on-target” “off tissue” toxicity, and unwanted target sin, while still demonstrating strong anti-tumor efficacy. Importantly, the PD1 Ab-IL-2 VitoKine's compatibility with higher dosages ensures the antibody arm can optimally target and reverse T-cell anergy and exhaustion, potentiating existing immune responses. This will result in further enhancement of the immune system's activity against tumors. Additionally, incorporating IL-2 with reduced potency, achieved by introducing mutations that disrupt IL-2Rβ or γc interaction, results in VitoKines with lower intrinsic basal activity and post-activation activity. This could potentially further broaden the therapeutic margin.

### Example 17

### PD1 Ab-IL-2 VitoKines' In Vivo Activity Hinges on Proteolytic Activation and is Reliant on PD1 Targeting

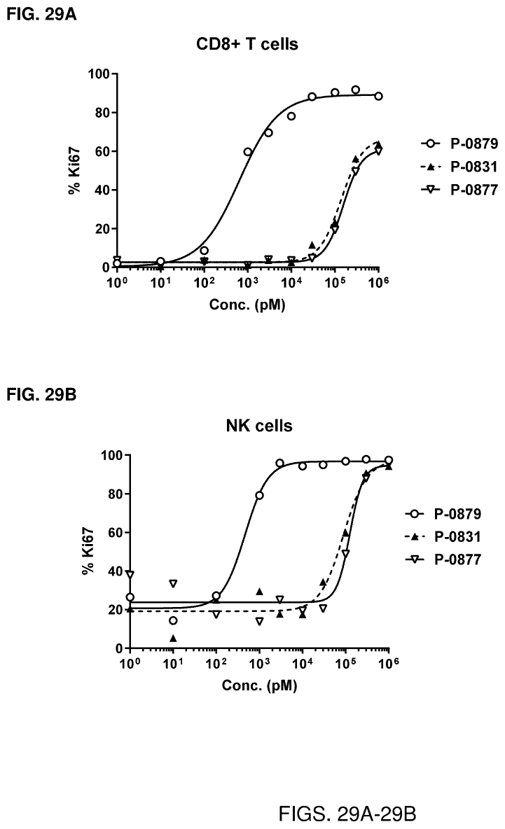

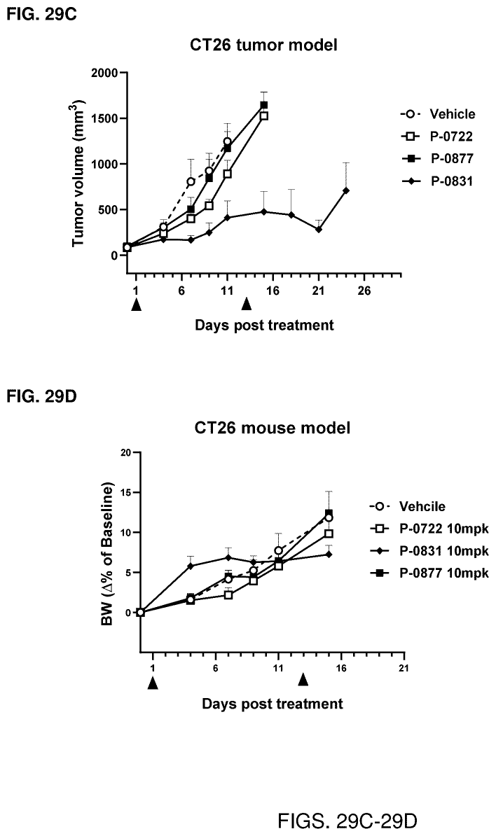

The critical role of the VitoKine activation in its anti-tumor efficacy was studied by comparing P-0831 and its non-cleavable VitoKine counterpart P-0877, using the murine CT26 colon carcinoma tumor model. The only difference between P-0831 and P-0877 lies in the L2 linker connecting IL-2 (D2) and IL-2Rα (D3) domains (refers to table 21 B for details). While P-0877 displayed identical activity as P-0831 in inducing Ki67 expression in CD8+ T and NK cells in human PBMCs, as depicted in FIGS. 29A and 29B, the IL-2 domain in P-0877 remains concealed and inactive as the D3 domain cannot be cleaved leading to activation.

For this study, female Balb/C mice, aged 7-9 weeks, were subcutaneously injected on the right flank with 5×10¹ CT26 cells. On the 11^{th} day, when the average tumor volume reached approximately 75 mm³, the mice were randomized into five groups, each containing eight animals. They received two Q12D intraperitoneal injections of a vehicle (PBS), or P-0722, P-0831, P-0877 at 10 mg/kg starting on the first Study Day, which was the next day following randomization. Tumor size and body weight were monitored twice weekly. Based on the established criteria, if a tumor grew to or exceeded 1500 mm³, or it became necrotic, the mouse was euthanized.

The CT26 syngeneic tumor model is generally less responsive to PD1 therapy compared to MC38 model. As depicted in FIG. 29C, tumors eventually developed in all mice. When treated with the mouse PD1 antibody P-0722, there was only a slight delay in tumor growth, resulting in a 25% tumor growth inhibition (TGI). On the other hand, administering P-0831 at the same dosage demonstrated a markedly improved efficacy, showing an 81% TGI. In a sharp contrast, P-0877, which has the non-activable inert IL-2 domain, did not display any improvement in inhibiting tumor growth compared to P-0722. These findings suggest that the enhanced anti-tumor effectiveness of VitoKine molecules hinges on the enzymatic cleavage of the linker to release the concealing moiety, thereby activating the IL-2 domain around the tumor. It is worth noting that all the tested compounds, when given at 10 mg/kg, were well-tolerated with no evidence of weight loss in mice as shown in FIG. 29D.

In a concurrently conducted study using the CT26 tumor model, the importance of PD1-targeting for the anti-tumor effectiveness of IL-2 VitoKine was assessed. This was done by comparing P-0871, a non-targeting IL-2 VitoKine, with P-0831. Both P-0871 and P-0831 share the same D2 and D3 domains as well as the L1 and L2 linkers, but P-0871's D1 domain is a non-targeting germline antibody P-1260, having heterodimeric heavy chains and a light chain with SEQ ID NOS: 191, 192, and 193.

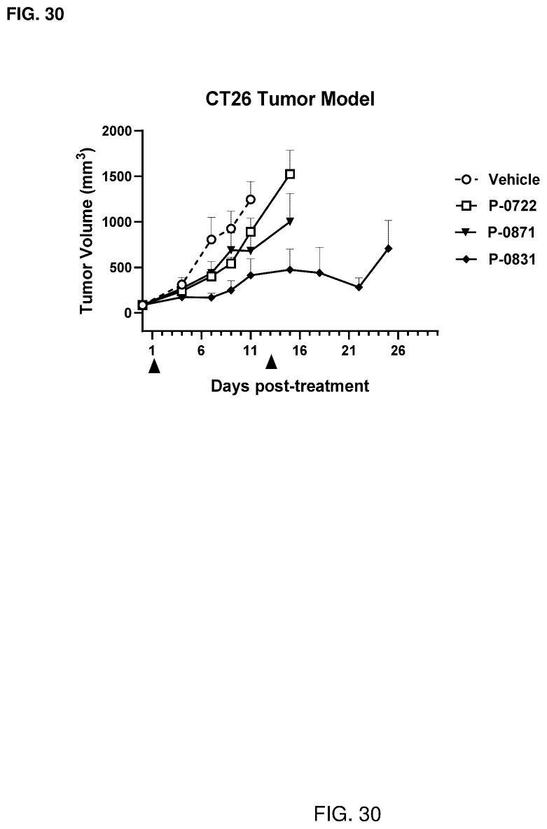

Mice bearing subcutaneously implanted CT26 tumors were administered two Q12D intraperitoneal injections of either a vehicle (PBS), P-0722, P-0831, or P-0871 at a dosage of 10 mg/kg. FIG. 30 depicts the mean tumor volumes (along with SEM) for each group as a function of time. The results show that P-0831 was markedly more effective in delaying tumor growth compared to its non-targeted counterpart, P-0871. This suggests that targeting PD1 is crucial in enhancing the anti-tumor potency of P-0831.

In conclusion, the findings strongly suggest that the anti-tumor effects of PD1 Ab-IL-2 VitoKine hinge on the in vivo proteolytic cleavage process, which subsequently leads to the activation of IL-2. Moreover, the effectiveness of this VitoKine appears to be closely tied to its ability to target PD1, indicating that PD1-targeting plays an important role in its therapeutic potential.

### Example 18

### Construction and Ex Vivo Characterization of PD1 Ab-IL-2 Immunocytokines

Tethering an IL-2 variant to an PD1 antibody aims to deliver the IL-2 variant preferentially in cis to PD1+ cells, such as activated and exhausted CD8+ T in tumor microenvironment, facilitating selective signaling. This strategy also reduces systemic exposure of IL-2 and can provide synergy by removing the negative regulation and reinvigorating T cells in both function and number. In addition to the VitoKine platform, using IL-2 variants with reduced/abolished binding to IL-2Rα and attenuated IL-2Rβγ activity offers an alternative approach to balance the proportion between the cytokine and antibody arms exhibiting dramatically different potency and molecular weights in their native versions. This balance allows for optimal dosing and preserves function of each arm. Diminished cytokine activity is expected to minimize peripheral activation, mitigate antigen-sink and target-mediated deposition in vivo, and promote tumor targeting via the antibody arm.

The PD1 antibodies used to construct PD1 Ab-IL-2 immunocytokines were selected from the optimized human PD1 blocking antibodies comprising light chain sequences set forth in SEQ ID NO: 44 and heavy chain sequences set forth in SEQ ID NOS: 45-49. These optimized PD1 blocking antibodies have a high affinity for the human PD1 protein and demonstrate equal or comparable potency as pembrolizumab in blocking PD1. They also possess a higher sequence similarity score to the closest human germline sequence, resulting in an improved degree of humanness compared to pembrolizumab. Furthermore, they are predicted to have lower hydrophobicity, which in turn is likely to lower their aggregation propensity than pembrolizumab. PD1-targeted IL-2 immunocytokines constructed using these optimized PD1 blocking antibodies are also projected to have enhanced developability profiles.

When creating PD1 Ab monomeric IL-2 immunocytokine fusions, an IL-2 variant is fused via a peptide linker to the C-terminus of a knob-containing heterodimeric heavy chain of an PD1 antibody. The human IgG1 knob-into-hole heavy chain pairs also comprise L234A, L235A, G237A mutations to abolish binding to FcγR and C1q, but retain FcRn binding for pharmacokinetics (PK). The Structure of PD1 Ab-IL-2 immunocytokine is depicted in FIG. 3B and exemplary immunocytokines are listed in Table 24.

TABLE 24
Exemplary human PD1 Ab-IL-2 immunocytokines

| PD1 | | | IL-2 |
|---|---|---|---|
| Ab-IL-2 | | | variant |
| immuno- | Immunocytokine | | SEQ |
| cytokine | SEQ ID NOS: | IL-2 mutations | ID NO: |
| P-1300 | 215 + 203 + 44 | P65R/C125I | 118 |
| P-1301 | 210 + 203 + 44 | L19Q/P65R/C125I | 152 |
| P-1302 | 211 + 203 + 44 | L19H/P65R/C125I | 152 |
| P-1303 | 212 + 203 + 44 | L19Q/P65R/C125I/Q126H | 174 |
| P-1391 | 213 + 203 + 44 | P65R/C125I/Q126G | 159 |
| P-1392 | 214 + 203 + 44 | P65R/C125I/Q126S | 168 |

As can be appreciated by skilled artisan in the field, any optimized PD1 antibodies disclosed in the present invention, including those with sequences set forth in seq ID NOS: 44-49, can be used to construct PD1 Ab-IL-2 immunocytokines, which is within the spirit and scope of the invention. Likewise, any IL-2 variants with varying degrees of potency reduction disclosed in this invention, especially those with sequences set forth in SEQ ID NOS:151-180, can serve as the building block to construct PD1-targeted IL-2 immunocytokines. These designs aims to potentiate and/or augment PD1 antibody-based therapies for a range of cancers.

All genes were codon optimized for expression in mammalian cells, synthesized, and subsequently subcloned into the recipient mammalian expression vector through the service of GenScript. The constructs were produced by co-transfecting ExpiCHO cells (ThermoFisher) with the said expression vectors, following manufacturer's instructions. Protein purification and characterization were conducted following the same procedures detailed in Example 2.

As anticipated, the potency of these IL-2 variants, originally observed in the Fc fusion format, was faithfully preserved when integrated with the PD1 antibody, as demonstrated in a human PBMC assay measuring Ki67 expression. Furthermore, these exemplary immunocytokines listed in Table 24 maintained the binding and PD1-blocking activities of their component PD1 antibodies, P-1271 (SEQ ID NOS: 49 and 44), as confirmed in the Promega PD1/PD-L1 blocking reporter assay.

Mouse PD1 Ab-IL-2 immunocytokines were produced analogously for in vivo tumor models in immunocompetent mice. IL-2 variants was fused to the C-terminus of anti-mouse PD1 HC chain 2 (SEQ ID NO: 190) of the heterodimeric heavy chain pair (SEQ ID NOS: 189 and 190) via a GS linker (SEQ ID NO: 114). The light chain of the mouse PD1 antibody has the sequence set forth in SEQ ID NO: 52. The exemplary mouse PD1 Ab IL-2 immunocytokines, specifically P-0782, P-0786, and P-0783, feature IL-2 variants with the P65R/C125I mutation (SEQ ID NO: 118), the L19Q/P65R/C125I mutation (SEQ ID NO: 152), and the L19H/P65R/C125I mutation (SEQ ID NO: 151), respectively. P-0837, containing an IL-2 domain of SEQ ID NO: 117, serves as the wild-type IL-2 immunocytokine control.

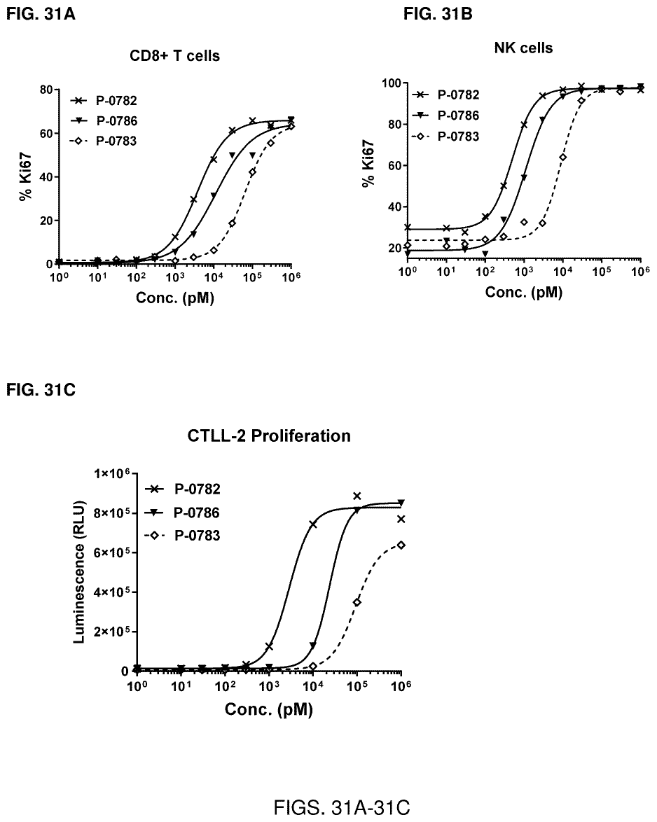

P-0782, P-0786, and P-0783 were subsequently assessed for their activity in stimulating Ki67 expression in CD8+ T and NK cells using human PBMCs. The degrees of potency attenuation in P-0786 and P-0783 compared to P-0782 (shown in FIGS. 31A and 31B), resulting from the incorporation of L19 mutations, paralleled their respective Fc fusion counterparts P-0759 and P-0731 versus P-0704, as illustrated in FIGS. 18A and 18B.

The proliferation activity of P-0782, P-0786, and P-0783 was further assessed using CTLL-2 cells, which are cytotoxic T cells derived from the C57BL/6 mouse. Briefly, CTLL2 cells were harvested, washed, and re-suspended in an IL-2-free medium (RPM11640, 10% FCS, 2 mM Glutamine) for a two-hour starvation period. Post-starvation, these cells, at 50,000/mL, were transferred into a 96-well U-bottom plate. Serial dilutions of the PD1 Ab-IL-2 immunocytokines were then added, followed by a two-day incubation. Cell proliferation was assessed using CellTiter-Glo (Promega) according to manufacturer's instructions, and luminescence signals were measured. As depicted in FIG. 31C, the potency reduction caused by the L19Q in P-0786 and the L19H mutation in P-0783, when compared to P-0782, was consistent between mouse-derived and human primary cells. This consistency underscores the mouse as a reliable model for analyzing the impact of IL-2 potency change on in vivo pharmacodynamics and anti-tumor efficacy.

### Example 19

### Pharmacodynamic Effects of PD1 Ab IL-2 Immunocytokines in Mice

The pharmacodynamic effects of the mouse PD1 Ab-IL-2 immunocytokines were assessed in C57BL/6 mice using a single dose administration. Seven-week-old female C57BL/6 mice from Charles River Laboratory were allowed a 7-day acclimation period before the start of the study. On Day 0, mice were given an intraperitoneal injection of either a vehicle or one of the test compounds: P-0837, P-0782, P-0783, or P-0786. Blood samples were withdrawn on Days 0, 3, 5, 7 and 10 post-injection. Each group consisted of 5 mice. The immune profiling of the heparin-treated whole blood was carried out following the procedure outlined in Example 14.

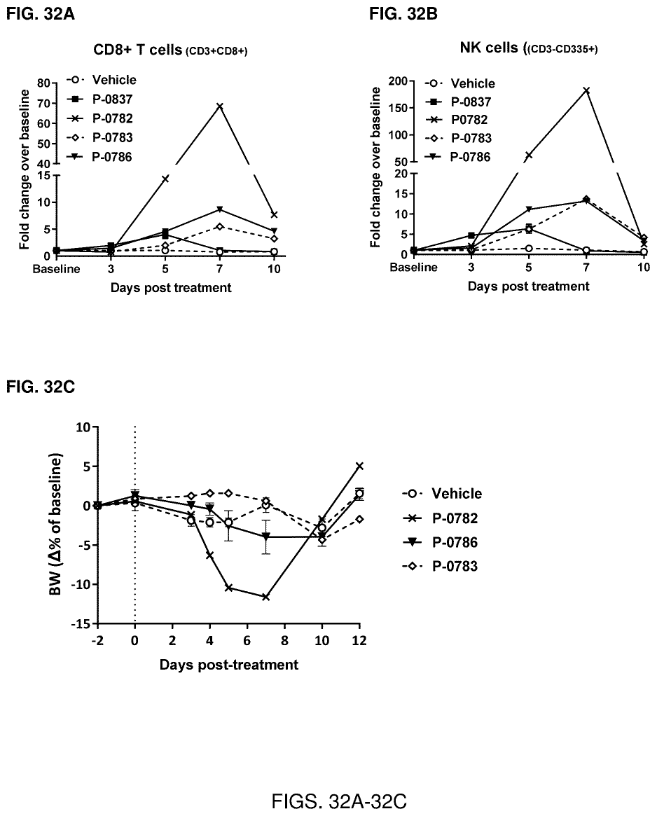

Following a single injection at 2 mg/kg, drastic differences in CD8 and NK cell expansion were observed among the test compounds. P-0782, containing a mutation that abolishes IL-2Rα binding but does not impact IL-2Rβγ interaction, exhibited vigorous expansion of CD8+ T (FIG. 32A) and NK cells (FIG. 32B). The expansion of these lymphocyte subsets started on Day 3, reaching a peak on Day 7 with an increase of 68-fold in CD8+ T cells and a dramatic 182-fold in NK cells. In a sharp contrast, P-0837, acting as the wild-type control, displayed a much milder response. The peak of cell expansion for both lymphocytes occurred on Day 5 with a more modest increase of 3.9-fold for CD8+ T cells and 6.8-fold for NK cells, as illustrated in FIGS. 32A and 32B.

The mutation that abrogates IL-2Rα binding could potentially minimize the IL-2Rα (CD25) sink effect, subsequently increasing the availability to IL-2Rβγ. As shown in P-0782, an IL-2Rβγ-selective full agonist, this enriched receptor engagement elicits vigorous expansion of cytotoxic cells. It is hypothesized that IL-2 mutations designed to reduce but not abolish IL-2Rα binding can fine-tune the response of regulatory T cells (as depicted in FIG. 15B). A right amount of modulation in IL-2Rα binding could establish an immune counterbalance to improve systemic tolerance without compromising tumor killing efficacy.

FIGS. 32A and 32B further display the pharmacodynamics of P-0783 and P-0786 following a 2 mg/kg dosage. Compared to P-0782, the maximal responses observed with P-0786 and P-0783 were notably reduced, in line with their overall attenuated potency. However, in contrast to P-0837, P-0786 exhibited a markedly prolonged and enhanced dose-responsive effect on cell expansion. Increases in both CD8+ T and NK cell numbers experienced a delay but were persistent and durable. The peak response was seen on Day 7, displaying an 8.6-fold increases for CD8+ T cells and 13-fold for NK cells. These numbers didn't return to the baseline level by Day 10. P-0783, comprising an even weaker IL-2 agonist, showed a similarly delayed but enduring effects, leading to expansion of 5.5-fold in CD8+ T cells and 14-fold in NK cells in a dose-dependent manner (FIGS. 32A and 32B).

Additionally, FIG. 32C highlights the direct correlation among the potency level, expansion of cytotoxic lymphocytes, and resultant body weight loss in mice. Specifically, P-0782, being an IL-2Rβγ-selective full agonist, induced dramatic surges in the numbers of both CD8+ T and NK cells, leading to the most substantial weight loss among the tested compounds. The potency-attenuated agonists, P-0786 and P-0783, showed enhanced in vivo tolerability. Of these, P-0783 appeared slightly more tolerable than P-0786, aligning with its characterization as a weaker agonist of the two.

In summary, P-0782 demonstrated a robust pharmacodynamic effect by significantly promoting the proliferation and expansion of CD8+ T and NK cells. While P-0786 and P-0783 displayed weaker effects, their responses were persistent. The in vitro and in vivo potency assessments of these compounds generally aligned. Notably, the attenuated potencies of P-0786 and P-0783 led to improve in vivo tolerability compared to the full-acting P-0782. The findings support our design premise that weakening cytokine potency helps alleviate pathway over-activation and mitigate antigen sink and target-mediated deposition, ultimately reducing toxicity and improving pharmacokinetic and pharmacodynamic outcomes.

### Example 20

### In Vivo Efficacy of PD1 Ab IL-2 Immunocytokines in Syngeneic Mouse Tumor Models

The anti-tumor efficacy of PD1 Ab-IL-2 immunocytokines, including P-0837, P-0782, P-0783, and P-0786, were investigated in the MC38 murine colon carcinoma model. Female C57BL/6 mice aged 7-9 weeks, were subcutaneously implanted with MC38 cells. Once tumor averaged ~75 mm³ in volume after about 2 weeks, the mice were randomized (n=8) and treated every 12 days (Q12D) with 0.5 mg/kg of the immunocytokines for 2 injections. A vehicle (PBS) was included as the control. Tumor size and mouse weight were monitored bi-weekly. Animals were euthanized if tumors reached/exceeded 1500 mm³ or became necrotic.

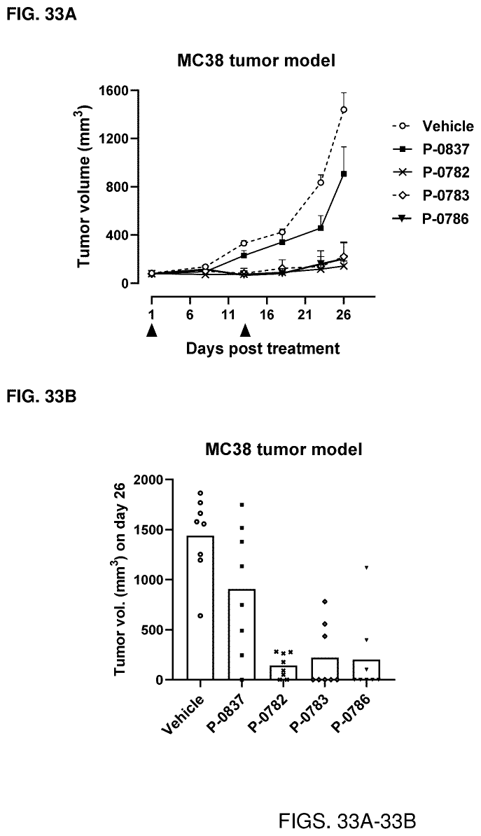

In FIG. 33A, the mean tumor volumes as a function of time along with the standard error of the mean (SEM) are presented for each group. The vehicle group (PBS-treated) showed rapid tumor growth. P-0837, a PD1 Ab immunocytokine with wild-type IL-2 equivalent, only inhibited tumor growth by 37% compared to the vehicle group. However, other PD1 Ab-IL-2 immunocytokines, P-0782, P-0783, and P-0786, all significantly reduced tumor growth by 85-90% by Day 26 post-treatment. Further analysis of individual tumor volumes (represented by each dot) on Day 26 (FIG. 33B) revealed that despite similar tumor growth inhibition, P-0782 rendered only two mice tumor-free, while P-0783 and P-0786 had five tumor-free cases each. The latter two both have attenuated IL-2 potency. It is hypothesized that the full IL-2 agonist P-0782 might cause pathway over-activation, target-mediated deposition, activation induced cell death, and upregulation of inhibitory signals on T cells, ultimately leading to reduced anti-tumor potency in vivo. Therefore, only PD1 Ab-IL-2 immunocytokines with attenuated IL-2 will be further studied for in vivo anti-tumor efficacy.

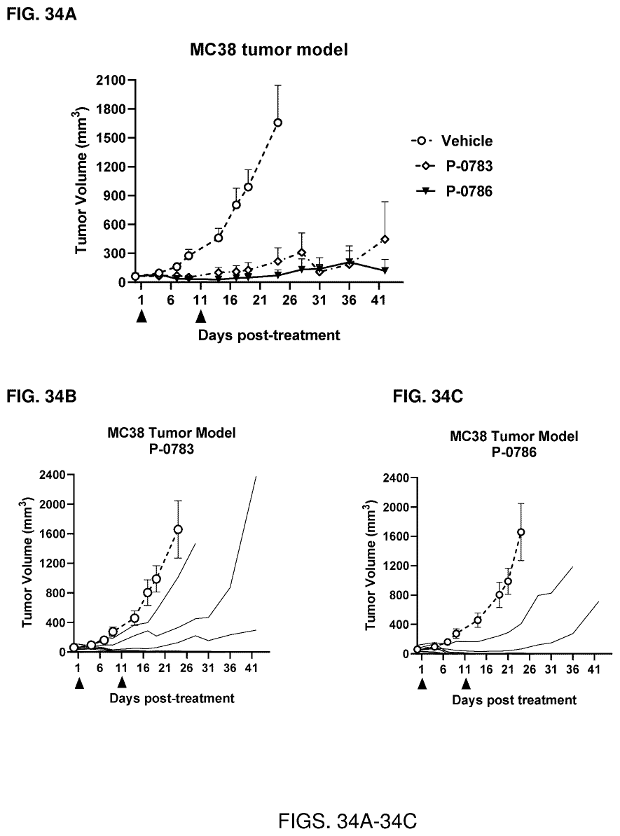

In a similarly conducted experiment illustrated in FIG. 34, both P-0783 and P-0786 showed significant tumor growth inhibition at a low dosage of 0.3 mg/kg (two Q10D doses). On Day 41, which is 30 days after the second and last treatment, 5 out of 8 mice treated with P-0783 and 6 out of 8 mice treated with P-0786 were free of tumor growth. At this dosage, P-0786 exhibited a slightly better anti-tumor effect compared to P-0783.

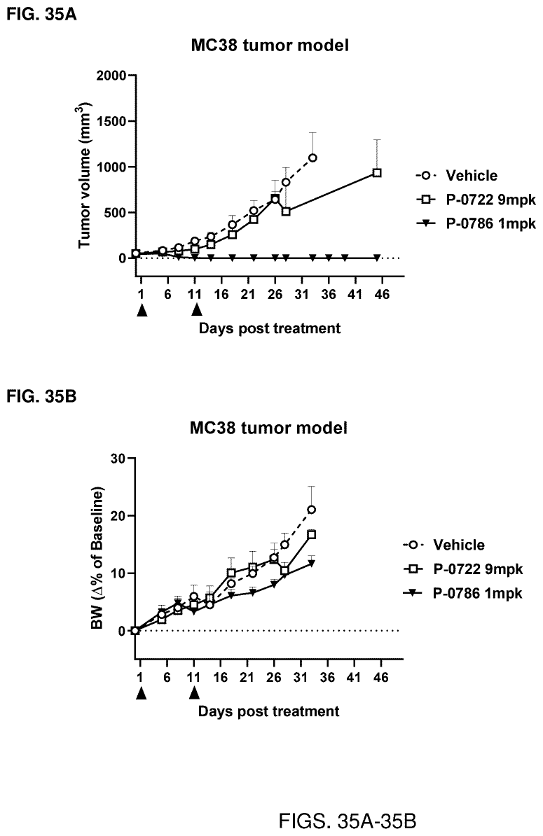

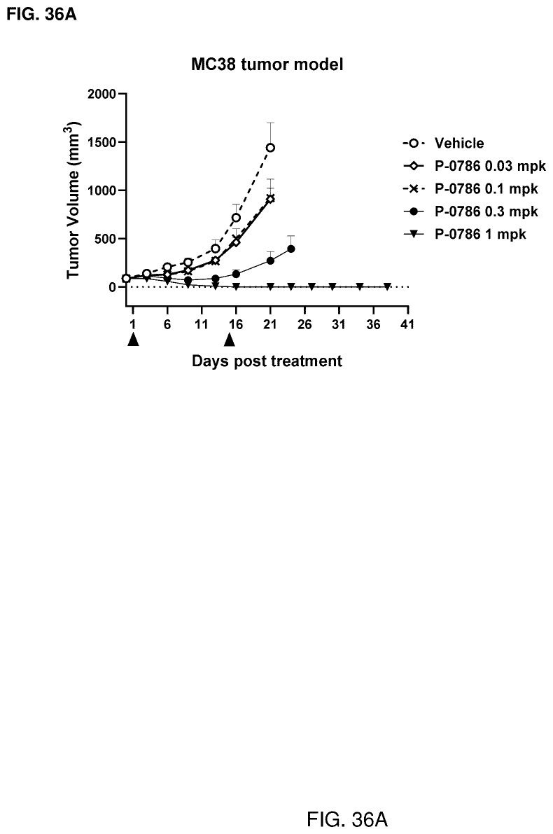

FIGS. 35A and 35B display the progression in tumor volumes and body weight changes over time in mice treated with two Q10D doses of either P-0782 or the mouse PD1 antibody P-0722. At a dosage of 9 mg/kg, P-0722 showed minimal efficacy. In a sharp contrast, even at a dosage reduced to 1/9^{th} (1 mg/kg) for P-0786, a pronounced and prolonged anti-tumor response was observed. By Day 45, which is 34 days after the final treatment, all treated mice with P-0786 showed no tumor growth. Moreover, there was no notable body weight reduction observed with P-0786 at 1 mg/g (FIG. 35B). These findings underscore the pivotal role of the IL-2 component in enhancing the anti-tumor effectiveness of the PD1 Ab-IL-2 immunocytokine.

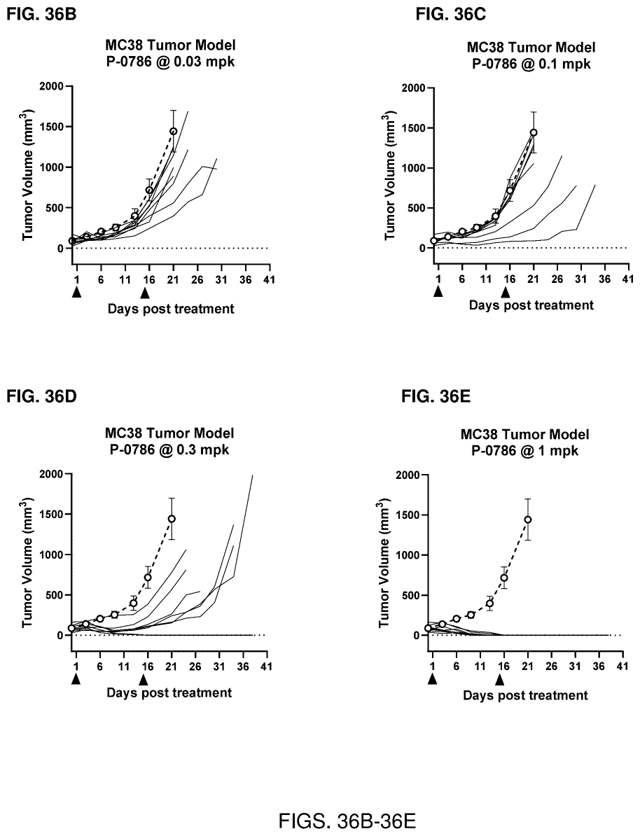

Further, the dose-response in MC38 tumor growth inhibition by P-0786 was evaluated. Mice with established MC38 tumors (with an average tumor volume of ~75 mm³ and n=8) received two Q14D doses of P-0786 at 0.03, 0.1, 0.3, and 1 mg/kg intraperitoneally. FIG. 36A displays the mean tumor volumes ±SEM for each group over time and FIGS. 36B to 36E illustrates individual tumor growth curves for each dose group. The average tumor size ±SEM of the vehicle group (represented by the dotted line) is displayed for comparison. Both the 0.03 and 0.1 mg/kg doses resulted in a modest 36% TGI, with no mice exhibiting complete tumor regression by Day 21. However, as the doses ascended from 0.1 to 1 mg/kg in roughly 3-fold increments, a pronounced dose response emerged. At 0.3 mg/kg, an 81% TGI was achieved on Day 21, with 2 out of 8 mice being tumor-free. Impressively, at the 1 mg/kg dosage, all 8 mice showed complete tumor eradication.

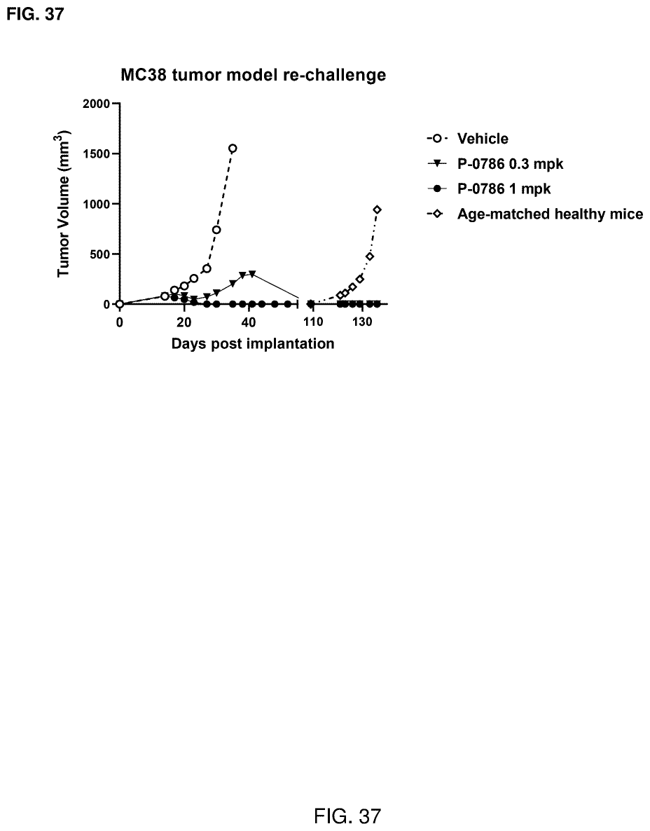

The 10 mice showed no tumor growth (2 from the 0.3 mg/kg dose and 8 from the 1 mg/kg dose) underwent a rechallenge implantation of MC38 cells on the 109^{th} days after the initial implantation, or 94 days after the first P-0786 dose. FIG. 37 reveals that none of these rechallenged mice had a tumor recurrence, unlike the age-matched naïve mice used as controls, which successfully developed tumors. These findings suggest that the PD1 Ab IL-2 immunocytokine successfully induced long-term immunity.

The efficacy of P-0786 was also evaluated in two other syngeneic tumor models: murine CT26 colon carcinoma and B16F10 murine melanoma models. In the CT26 model, female Balb/C mice were implanted subcutaneously with 5×10⁵ CT26 cells, while the B16F10 model was similarly established by implanting 5×10⁵ B16F10 cells into female C57BL/6 mice. CT26 tumor-bearing mice received two doses of P-0786 (0.6 mg/kg and 2 mg/kg) every 12 days, while B16F10 tumor-bearing mice received same dosages every 10 days. All mice were regularly monitored with bi-weekly tumor measurements.

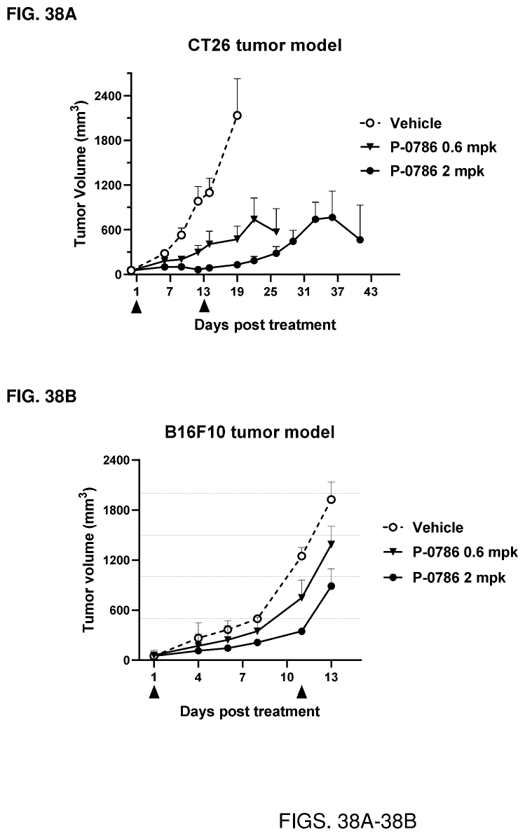

FIG. 38 reveals P-0786's dose-dependent, single-agent anti-tumor effects in both CT26 (FIG. 38A) and B16F10 (FIG. 38B) models. In the CT26 model, there was strong tumor growth inhibition for both doses (65% TGI at 0.6 mg/kg and 91% TGI at 2 mg/kg by Day 21). By the 41^{st} day after the first treatment, one out of 7 mice in the 0.6 mg/kg group and 2 out of 7 mice in the 2 mg/kg group were tumor-free. The B16F10 model, which grows aggressively and is less responsive to PD1 therapy than MC38 mode, showed initial tumor growth delay with P-0786 (35% TGI at 0.6 mg/kg and 58% TGI for 2 mg/kg), but tumors eventually developed in all mice (FIG. 38B).

In summary, the PD1 Ab-IL-2 immunocytokine effectively suppressed tumor growth across multiple syngeneic mouse tumor models. When compared to the IL-2Rβγ-selective full agonist, the potency-attenuated IL-2, achieved by disrupting IL-2Rβ interaction demonstrated improved in vivo tolerability and enhanced single-agent anti-tumor efficacy. It is predicted that PD1 Ab IL-2 immunocytokine featuring IL-2 mutations that interfere γc interaction would have similar improvement. Given the distinct expression profile of γc in peripheral cells, IL-2 variants with weakened γc activity may off the added benefits of reduced target sink and enhanced bioavailability.

All of the articles and methods disclosed and claimed herein can be made and executed without undue experimentation in light of the present disclosure. While the articles and methods of this invention have been described in terms of preferred embodiments, it will be apparent to those of skill in the art that variations may be applied to the articles and methods without departing from the spirit and scope of the invention. All such variations and equivalents apparent to those skilled in the art, whether now existing or later developed, are deemed to be within the spirit and scope of the invention as defined by the appended claims. All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference in its entirety for any and all purposes. The invention illustratively described herein suitably may be practiced in the absence of any element(s) not specifically disclosed herein. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### SEQUENCE LISTINGS

The amino acid sequences listed in the accompanying sequence listing are shown using standard letter abbreviations for nucleotide bases and one letter codes for amino acids, as defined in 37 C.F.R. 1.822.

SEQ ID NO: 1 is the amino acid sequence of a mature human PD1 polypeptide.

SEQ ID NOS: 2-5 are the amino acid sequences of human PD1 blocking antibody light chain variable domains.

SEQ ID NOS: 6-18 are the amino acid sequences of human PD1 blocking antibody heavy chain variable domains.

SEQ ID NOS: 19-21 are the amino acid sequences of human PD1 blocking antibody light chain CDR1.

SEQ ID NOS: 22-24 are the amino acid sequences of human PD1 blocking antibody light chain CDR2.

SEQ ID NO: 25 is the amino acid sequence of human PD1 blocking antibody light chain CDR3.

SEQ ID NO: 26 is the amino acid sequence of human PD1 blocking antibody heavy chain CDR1.

SEQ ID NOS: 27-32 are the amino acid sequences of human PD1 blocking antibody heavy chain CDR2.

SEQ ID NO: 33 is the amino acid sequence of human PD1 blocking antibody heavy chain CDR3.

SEQ ID NO: 34 is the amino acid sequence of human kappa light chain constant domain.

SEQ ID NO: 35 is the amino acid sequence of human IgG1 heavy chain constant domain comprising L234A/L235A/G237A mutations.

SEQ ID NO: 36 is the amino acid sequence of human IgG4 heavy chain constant domain comprising S228P mutation.

SEQ ID NO: 37 is the amino acid sequence of human immunoglobulin germline exon HGHV1-2 (GenBank accession NO: X62106).

SEQ ID NO: 38 is the amino acid sequence of human immunoglobulin germline exon HGHV3-23 (GenBank accession NO: M99660).

SEQ ID NO: 39 is the amino acid sequence of human immunoglobulin germline exon HGKV3D-11 (GenBank accession NO: X17264).

SEQ ID NO: 40 is the amino acid sequence of human antibody heavy chain variable domain with GenBank accession NO: AB063829.

SEQ ID NO: 41 is the amino acid sequence of human antibody light chain variable domain with GenBank accession NO: M29469.

SEQ ID NO: 42 is the amino acid sequence of the light chain of reference human PD1 blocking antibody P-0734.

SEQ ID NO: 43 is the amino acid sequence of the heavy chain of reference human PD1 blocking antibody P-0734.

SEQ ID NO: 44 is the amino acid sequence of the light chain of human PD1 blocking antibodies.

SEQ ID NO: 45 is the amino acid sequence of the heavy chain of human PD1 blocking antibody P-1174.

SEQ ID NO: 46 is the amino acid sequence of the heavy chain of human PD1 blocking antibody P-1194.

SEQ ID NO: 47 is the amino acid sequence of the heavy chain of human PD1 blocking antibody P-1201.

SEQ ID NO: 48 is the amino acid sequence of the heavy chain of human PD1 blocking antibody P-1238.

SEQ ID NO: 49 is the amino acid sequence of the heavy chain of PD1 human blocking antibody P-1271.

SEQ ID NO: 50 is the amino acid sequence of the light chain of a benchmark human PD1 blocking antibody P-0795.

SEQ ID NO: 51 is the amino acid sequence of the heavy chain of a benchmark human PD1 blocking antibody P-0795.

SEQ ID NO: 52 is the amino acid sequence of the light chain of a surrogate mouse PD1 blocking antibody P-0722.

SEQ ID NO: 53 is the amino acid sequence of the heavy chain of a surrogate mouse PD1 blocking antibody P-0722.

SEQ ID SEQ ID NOS: 54-77 are the amino acid sequences of various protease substrate peptides.

SEQ ID NOS: 78-94 are the amino acid sequences of various protease cleavable linkers comprising various spacer peptides flanking protease substrate peptides.

SEQ ID NOS: 95-115 are the amino acid sequences of various non-cleavable linker sequences.

SEQ ID NO: 116 is a human IL-2 mature form amino acid sequence.

SEQ ID NOS: 117-180 are the amino acid sequences of human IL-2 variant polypeptides.

SEQ ID NO: 181 is a human IL-2Rα amino acid sequence.

SEQ ID NO: 182 is a human IL-2Rαsushi domain amino acid sequence.

SEQ ID NOS: 183-185 are the amino acid sequences of human IL-2Rαsushi domain variant polypeptides.

SEQ ID NO: 186 is the amino acid sequence of a human IgG1 Fc comprising L234A/L235A/G237A mutations.

SEQ ID NO: 187 is the amino acid sequence of a human IgG1 Knob-Fc comprising L234A/L235A/G237A mutations.

SEQ ID NO: 188 is the amino acid sequence of a human IgG1 Hole-Fc comprising L234A/L235A/G237A mutations.

SEQ ID NOS: 189 and 190 are the amino acid sequences of the heterodimeric heavy chains of a surrogate mouse PD1 Ab P-0722.

SEQ ID NOS: 191 and 192 are the amino acid sequences of the heterodimeric heavy chains of a germline antibody P-1260.

SEQ ID NOS: 193 is the amino acid sequence of the light chain of a germline antibody P-1260.

SEQ ID NOS: 194-209 are the amino acid sequences of the heavy chains of various human PD1 Abs and/or human PD1 Ab-IL-2 VitoKines.

SEQ ID NOS: 210-215 are the amino acid sequences of the heavy chains of various human PD1 Ab-IL-2 Immunocytokines.

1-43. (canceled)

44. An isolated Interleukin-2 (IL-2) fusion protein complex comprising an IL-2 polypeptide (or variant thereof) linked to an PD1 blocking antibody to form an IL-2-PD1 blocking antibody fusion protein, wherein the PD1 blocking antibody is selected from an antibody comprising: (a) a light chain variable region comprising amino acids having the sequence set forth in SEQ ID NO: 3, and a heavy chain variable region comprising amino acids having the sequence set forth in SEQ ID NO: 7; or comprising (b) a light chain variable region comprising amino acids having the sequence set forth in SEQ ID NO: 3, and a heavy chain variable region comprising amino acids having the sequence set forth in SEQ ID NO: 9; or comprising (c) a light chain variable region comprising amino acids having the sequence set forth in SEQ ID NO: 3, and a heavy chain variable region comprising amino acids having the sequence set forth in SEQ ID NO: 11; or comprising (d) a light chain variable region comprising amino acids having the sequence set forth in SEQ ID NO: 3, and a heavy chain variable region comprising amino acids having the sequence set forth in SEQ ID NO: 13; or comprising (e) a light chain variable region comprising amino acids having the sequence set forth in SEQ ID NO: 3, and a heavy chain variable region comprising amino acids having the sequence set forth in SEQ ID NO: 18, and wherein the PD1 blocking antibody targets the IL-2-PD1 blocking antibody fusion protein to tumor-infiltrating lymphocytes (TILs).

45. The IL-2-PD1 blocking antibody fusion protein according to claim 44, wherein the PD1 blocking antibody is selected from an antibody comprising: (a) the light chain sequence set forth in SEQ ID NO: 44, and the heavy chain sequence set forth in SEQ ID NO: 45; or comprising (b) the light chain sequence set forth in SEQ ID NO: 44, and the heavy chain sequence set forth in SEQ ID NO: 46; or comprising (c) the light chain sequence set forth in SEQ ID NO: 44, and the heavy chain sequence set forth in SEQ ID NO: 47; or the light chain sequence set forth in SEQ ID NO: 44; or comprising (d) the heavy chain sequence set forth in SEQ ID NO: 48; or comprising (e) the light chain sequence set forth in SEQ ID NO: 44, and the heavy chain sequence set forth in SEQ ID NO: 49.

46. The bioactivatable polypeptide drug construct according to claim 44, wherein the IL-2 variant polypeptide is an IL-2 variant polypeptide selected from the group of polypeptides having the amino acid sequence set forth in SEQ ID NOs: 118-125 and 151-180.

47. The bioactivatable polypeptide drug construct according to claim 46, wherein the IL-2 variant polypeptide is an IL-2 variant polypeptide selected from: (a) a polypeptide having the amino acid sequence set forth in SEQ ID NO: 118; (b) a polypeptide having the amino acid sequence set forth in SEQ ID NO: 151; a polypeptide having the amino acid sequence set forth in SEQ ID NO: 152.

48. The IL-2-PD1 blocking antibody fusion protein according to claim 44, wherein the IL-2 polypeptide is covalently attached to the PD1 blocking antibody by a peptide linker wherein the peptide linker is selected from the group of sequences set forth in SEQ ID NOs: 54-115.

49. A pharmaceutical composition comprising a construct according to claim 44 in admixture with a pharmaceutically acceptable carrier.

50. A method for treating cancer or cancer metastasis in a subject, comprising administering to the subject a therapeutically effective amount of the pharmaceutical composition according to claim 49.

51. The method according to claim 50, wherein the cancer is selected from pancreatic cancer, gastric cancer, liver cancer, breast cancer, ovarian cancer, colorectal cancer, melanoma, leukemia, myelodysplastic syndrome, lung cancer, prostate cancer, brain cancer, bladder cancer, head-neck cancer, or rhabdomyosarcoma or any cancer.

52. A bioactivatable polypeptide drug construct comprising, in an N- to C-terminal direction (D1-D2-D3): 1) a tumor-infiltrating lymphocyte (TIL)-targeting moiety D1 domain (“D1”), 2) a bioactivatable moiety D2 domain (“D2”), and 3) a concealing moiety D3 domain (“D3”); wherein D1 functions to target the bioactivatable moiety to the intended site of therapy; wherein D3 is capable of concealing the functional activity of D2 until activated at the intended site of therapy; wherein D1 is an PD1 blocking antibody, wherein D2 is an IL-2 variant polypeptide, and wherein D3 is an IL-2Rα sushi variant.

53. The bioactivatable polypeptide drug construct according to claim 52, wherein the PD1 blocking antibody is selected from an antibody which comprises: (a) a light chain variable region comprising amino acids having the sequence set forth in SEQ ID NO: 3, and a heavy chain variable region comprising amino acids having the sequence set forth in SEQ ID NO: 7; or (b) a light chain variable region comprising amino acids having the sequence set forth in SEQ ID NO: 3, and a heavy chain variable region comprising amino acids having the sequence set forth in SEQ ID NO: 9; or (c) a light chain variable region comprising amino acids having the sequence set forth in SEQ ID NO: 3, and a heavy chain variable region comprising amino acids having the sequence set forth in SEQ ID NO: 11; (d) a light chain variable region comprising amino acids having the sequence set forth in SEQ ID NO: 3, and a heavy chain variable region comprising amino acids having the sequence set forth in SEQ ID NO: 13; or (e) a light chain variable region comprising amino acids having the sequence set forth in SEQ ID NO: 3, and a heavy chain variable region comprising amino acids having the sequence set forth in SEQ ID NO: 18.

54. The bioactivatable polypeptide drug construct according to claim 52, wherein the PD1 blocking antibody is selected from an antibody which comprises: (a) the light chain sequence set forth in SEQ ID NO: 44, and the heavy chain sequence set forth in SEQ ID NO: 45; or (b) the light chain sequence set forth in SEQ ID NO: 44, and the heavy chain sequence set forth in SEQ ID NO: 46; or (c) the light chain sequence set forth in SEQ ID NO: 44, and the heavy chain sequence set forth in SEQ ID NO: 47; or the light chain sequence set forth in SEQ ID NO: 44; or (d) the heavy chain sequence set forth in SEQ ID NO: 48; and (e) the light chain sequence set forth in SEQ ID NO: 44, and the heavy chain sequence set forth in SEQ ID NO: 49.

55. The bioactivatable polypeptide drug construct according to claim 52, wherein domain D2 is an IL-2 variant polypeptide selected from the group of polypeptides having the amino acid sequence set forth in SEQ ID NOs: 118-125 and 151-180.

56. The bioactivatable polypeptide drug construct according to claim 55, wherein domain D2 is an IL-2 variant polypeptide selected from: (a) a polypeptide having the amino acid sequence set forth in SEQ ID NO: 118; (b) a polypeptide having the amino acid sequence set forth in SEQ ID NO: 151; a polypeptide having the amino acid sequence set forth in SEQ ID NO: 152.

57. The bioactivatable polypeptide drug construct according to claim 52, wherein domain D3 is an IL-2Rα sushi variant polypeptide selected from the group of polypeptides having the amino acid sequence set forth in SEQ ID NOs: 183-185.

58. The bioactivatable polypeptide drug construct according to claim 52, selected from the group of constructs wherein (a) the D1, D2 and D3 domains of the construct are each in the form of a monomer; and (b) the D1, D2 and D3 domains of the construct are each in the form of a dimer.

59. The bioactivatable polypeptide drug construct according to claim 52, wherein D2 is attached to D1 by a peptide linker (“L1”) selected from the group consisting of a protease cleavable peptide linker and a non-cleavable peptide linker, wherein the protease cleavable peptide linker is selected from the group of sequences set forth in SEQ ID NOs: 54-77 and 78-94, and wherein the non-cleavable peptide linker is selected from the group of sequences set forth in SEQ ID NOs: 95-115.

60. The bioactivatable polypeptide drug construct according to claim 52, wherein D2 is attached to D3 by a peptide linker (“L2”) selected from the group consisting of a protease cleavable peptide linker and a non-cleavable peptide linker, wherein the protease cleavable peptide linker is selected from the group of sequences set forth in SEQ ID NOs: 54-77 and 78-94, and wherein the non-cleavable peptide linker is selected from the group of sequences set forth in SEQ ID NOs: 95-115.

61. A pharmaceutical composition comprising a construct according to claim 52 in admixture with a pharmaceutically acceptable carrier.

62. A method for treating cancer or cancer metastasis in a subject, comprising administering to the subject a therapeutically effective amount of the pharmaceutical composition according to claim 61.

63. The method according to claim 62, wherein the cancer is selected from pancreatic cancer, gastric cancer, liver cancer, breast cancer, ovarian cancer, colorectal cancer, melanoma, leukemia, myelodysplastic syndrome, lung cancer, prostate cancer, brain cancer, bladder cancer, head-neck cancer, or rhabdomyosarcoma or any cancer.


SEQUENCE LISTING (215 sequences)

SEQ ID NO: 1 - protein, 530 aa, Homo sapiens
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPLFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 112 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 3 - protein, 112 aa, synthetic construct
EIVLTQSPATLSLSPGERATLSCRASQGVSTSGYSYLHWYQQKPGQAPRLLIYLASYRESGVPARFSGSGSGTDFTLTISSLEPEDFAVYYCQHSRDLPLTFGGGTKVEIKR

SEQ ID NO: 4 - protein, 112 aa, synthetic construct
EIVLTQSPATLSLSPGERATLSCRASQGVSTSGYSYLHWYQQKPGQAPRLLIYLASYRASGVPARFSGSGSGTDFTLTISSLEPEDFAVYYCQHSRDLPLTFGGGTKVEIKR

SEQ ID NO: 5 - protein, 112 aa, synthetic construct
EIVLTQSPATLSLSPGERATLSCRASQGVSTSGYSYLAWYQQKPGQAPRLLIYLASYRASGVPARFSGSGSGTDFTLTISSLEPEDFAVYYCQHSRDLPLTFGGGTKVEIKR

SEQ ID NO: 6 - protein, 120 aa, synthetic construct [sequence omitted]

SEQ ID NO: 7 - protein, 120 aa, synthetic construct
QVQLVQSGVEVKKPGASVKVSCKASGYTFTNYYMYWVRQAPGQGLEWMGGINPSNGGTNFAQKFQGRVTLTTDSSTTTAYMELKSLQFDDTAVYYCARRDYRFDMGFDYWGQGTTVTVSS

SEQ ID NO: 8 - protein, 120 aa, synthetic construct
QVQLVQSGVEVKKPGASVKVSCKASGYTFTNYYMYWVRQAPGQGLEWMGGINPSNGGTNYAQKFQGRVTLTTDSSTTTAYMELKSLQFDDTAVYYCARRDYRFDMGFDYWGQGTTVTVSS

SEQ ID NO: 9 - protein, 120 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSS

SEQ ID NO: 10 - protein, 120 aa, synthetic construct
EVQLLESGGGLVQPGGSLRLSCAASGFTFTNYYMYWVRQAPGKGLEWVSGINPSNGGTNYADKFKGRFTISRDNSKNTLYLQMNSLRAEDTAVYYCARRDYRFDMGFDYWGQGTLVTVSS

SEQ ID NO: 11 - protein, 120 aa, synthetic construct
EVQLLESGGGLVQPGGSLRLSCAASGFTFTNYYMYWVRQAPGKGLEWVSGINPSNGGTNYADKFKGRFTLSTDSSKNTLYLQMNSLRAEDTAVYYCARRDYRFDMGFDYWGQGTLVTVSS

SEQ ID NO: 12 - protein, 120 aa, synthetic construct
EVQLLESGGGLVQPGGSLRLSCAASGFTFTNYYMYWVRQAPGKGLEWMGGINPSNGGTNYADKFKGRFTISRDNSKNTLYLQMNSLRAEDTAVYYCARRDYRFDMGFDYWGQGTLVTVSS

SEQ ID NO: 13 - protein, 120 aa, synthetic construct
EVQLLESGGGLVQPGGSLRLSCAASGFTFTNYYMYWVRQAPGKGLEWMGGINPSNGGTNYADKFKGRFTLSTDSSKNTLYLQMNSLRAEDTAVYYCARRDYRFDMGFDYWGQGTLVTVSS

SEQ ID NO: 14 - protein, 120 aa, synthetic construct
EVQLLESGGGLVQPGGSLRLSCAASGFTFTNYYMYWVRQAPGKGLEWVSGINPSNGGTNFNDSVKGRFTISRDNSKNTLYLQMNSLRAEDTAVYYCARRDYRFDMGFDYWGQGTLVTVSS

SEQ ID NO: 15 - protein, 120 aa, synthetic construct
EVQLLESGGGLVQPGGSLRLSCAASGFTFTNYYMYWVRQAPGKGLEWVSGINPSNGGTNFADKFKGRFTISRDNSKNTLYLQMNSLRAEDTAVYYCARRDYRFDMGFDYWGQGTLVTVSS

SEQ ID NO: 16 - protein, 120 aa, synthetic construct
EVQLLESGGGLVQPGGSLRLSCAASGFTFTNYYMYWVRQAPGKGLEWVSGINPSNGGTNFADKFKGRFTISRDSSKNTLYLQMNSLRAEDTAVYYCARRDYRFDMGFDYWGQGTLVTVSS

SEQ ID NO: 17 - protein, 120 aa, synthetic construct
EVQLLESGGGLVQPGGSLRLSCAASGFTFTNYYMYWVRQAPGKGLEWVSGINPSNGGTNFADKFKGRFTISTDSSKNTLYLQMNSLRAEDTAVYYCARRDYRFDMGFDYWGQGTLVTVSS

SEQ ID NO: 18 - protein, 120 aa, synthetic construct
EVQLLESGGGLVQPGGSLRLSCAASGFTFTNYYMYWVRQAPGKGLEWVSGINPSNGGTNFADKFKGRFTLSTDSSKNTLYLQMNSLRAEDTAVYYCARRDYRFDMGFDYWGQGTLVTVSS

SEQ ID NO: 19 - protein, 15 aa, synthetic construct
RASKGVSTSGYSYLH

SEQ ID NO: 20 - protein, 15 aa, synthetic construct
RASQGVSTSGYSYLH

SEQ ID NO: 21 - protein, 15 aa, synthetic construct
RASQGVSTSGYSYLA

SEQ ID NO: 22 - protein, 8 aa, synthetic construct
YLASYLES

SEQ ID NO: 23 - protein, 8 aa, synthetic construct
YLASYRES

SEQ ID NO: 24 - protein, 8 aa, synthetic construct
YLASYRAS

SEQ ID NO: 25 - protein, 9 aa, synthetic construct
QHSRDLPLT

SEQ ID NO: 26 - protein, 5 aa, synthetic construct
NYYMY

SEQ ID NO: 27 - protein, 17 aa, synthetic construct
GINPSNGGTNFNEKFKN

SEQ ID NO: 28 - protein, 17 aa, synthetic construct
GINPSNGGTNFAQKFQG

SEQ ID NO: 29 - protein, 17 aa, synthetic construct
GINPSNGGTNYAQKFQG

SEQ ID NO: 30 - protein, 17 aa, synthetic construct
GINPSNGGTNYADKFKG

SEQ ID NO: 31 - protein, 17 aa, synthetic construct
GINPSNGGTNFADKFKG

SEQ ID NO: 32 - protein, 17 aa, synthetic construct
GINPSNGGTNFNDSVKG

SEQ ID NO: 33 - protein, 11 aa, synthetic construct
RDYRFDMGFDY

SEQ ID NO: 34 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 37 - protein, 98 aa, Homo sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAR

SEQ ID NO: 38 - protein, 98 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 39 - protein, 95 aa, Homo sapiens
EIVLTQSPATLSLSPGERATLSCRASQGVSSYLAWYQQKPGQAPRLLIYDASNRATGIPARFSGSGPGTDFTLTISSLEPEDFAVYYCQQRSNWH

SEQ ID NO: 40 - protein, 122 aa, Homo sapiens
QVQLVQSGVEVKKPGASVKVSCKASGYTFTSNAISWVRQAPGQGLEWMGWISTYKGKANYAQKFQDRVTLTTDSSTTTAYMELKSLQFDDTAVYYCARWRAVVGRGGGLDVWGQGTTVTVSS

SEQ ID NO: 41 - protein, 108 aa, Homo sapiens
EIVLTQSPATLSLSPGERATLSCRASQSVSSYLAWYQQKPGQAPRLLIYDASNKATGVPARFSGSGSGTDFTLTISSLEPEDFAVYYCQQSSKWPLTFGGGTKVEIKG

SEQ ID NO: 42 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 449 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 218 aa, synthetic construct
EIVLTQSPATLSLSPGERATLSCRASQGVSTSGYSYLHWYQQKPGQAPRLLIYLASYRESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 449 aa, synthetic construct
QVQLVQSGVEVKKPGASVKVSCKASGYTFTNYYMYWVRQAPGQGLEWMGGINPSNGGTNFAQKFQGRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 46 - protein, 449 aa, synthetic construct
EVQLLESGGGLVQPGGSLRLSCAASGFTFTNYYMYWVRQAPGKGLEWVSGINPSNGGTNYADKFKGRFTLSTDSSKNTLYLQMNSLRAEDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 449 aa, synthetic construct
EVQLLESGGGLVQPGGSLRLSCAASGFTFTNYYMYWVRQAPGKGLEWVSGINPSNGGTNFADKFKGRFTLSTDSSKNTLYLQMNSLRAEDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 449 aa, synthetic construct
EVQLLESGGGLVQPGGSLRLSCAASGFTFTNYYMYWVRQAPGKGLEWMGGINPSNGGTNYADKFKGRFTLSTDSSKNTLYLQMNSLRAEDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 449 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 214 aa, synthetic construct
DIVMTQSPLSLPVTPGEPASITCKASQDVETVVAWYLQKPGQSPRLLIYWASTRHTGVPDRFSGSGSGTDFTLKISRVEAEDVGVYYCQQYSRYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 446 aa, synthetic construct
EVQLVESGGGLVKPGGSLRLSCAASGFTFSSYDMSWVRQAPGKGLEWVATISGGGSYTYYPDSVKGRFTISRDNAKNSLYLQMNSLRAEDTAVYYCASPDSSGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - protein, 218 aa, Mus musculus
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKRTDAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 53 - protein, 444 aa, Mus musculus
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPG

SEQ ID NO: 54 - protein, 8 aa, synthetic construct
SPLGLAGS

SEQ ID NO: 55 - protein, 8 aa, synthetic construct
EPLELRAG

SEQ ID NO: 56 - protein, 8 aa, synthetic construct
LSGRSDNH

SEQ ID NO: 57 - protein, 8 aa, synthetic construct
GPLGIAGQ

SEQ ID NO: 58 - protein, 8 aa, synthetic construct
GTAHLMGG

SEQ ID NO: 59 - protein, 8 aa, synthetic construct
RIGSLRTA

SEQ ID NO: 60 - protein, 10 aa, synthetic construct
SGRSENIRTA

SEQ ID NO: 61 - protein, 8 aa, synthetic construct
GPLGMLSQ

SEQ ID NO: 62 - protein, 8 aa, synthetic construct
GPAGMKGL

SEQ ID NO: 63 - protein, 8 aa, synthetic construct
RPSASRSA

SEQ ID NO: 64 - protein, 6 aa, synthetic construct
PLGLAG

SEQ ID NO: 65 - protein, 13 aa, synthetic construct
LGGSGRSANAILE

SEQ ID NO: 66 - protein, 10 aa, synthetic construct
GGSGRSANAI

SEQ ID NO: 67 - protein, 5 aa, synthetic construct
SGRSA

SEQ ID NO: 68 - protein, 4 aa, synthetic construct
AANL

SEQ ID NO: 69 - protein, 7 aa, synthetic construct
GPTNKVR

SEQ ID NO: 70 - protein, 4 aa, synthetic construct
GFFY

SEQ ID NO: 71 - protein, 8 aa, synthetic construct
GPICFRLG

SEQ ID NO: 72 - protein, 7 aa, synthetic construct
RQAGFSL

SEQ ID NO: 73 - protein, 8 aa, synthetic construct
RQARAVGG

SEQ ID NO: 74 - protein, 5 aa, synthetic construct
PMAKK

SEQ ID NO: 75 - protein, 6 aa, synthetic construct
HSSKLQ

SEQ ID NO: 76 - protein, 13 aa, synthetic construct
GPLGMLSQPMAKK

SEQ ID NO: 77 - protein, 13 aa, synthetic construct
PMAKKGPLGMLSQ

SEQ ID NO: 78 - protein, 30 aa, synthetic construct
GGGSGGGGSGGGGSLSGRSDNHGGSGGGGS

SEQ ID NO: 79 - protein, 15 aa, synthetic construct
GSSSGRSENIRTAGT

SEQ ID NO: 80 - protein, 35 aa, synthetic construct
GGGGSGGGGSGGGSLGGSGRSANAILEGGSGGGGS

SEQ ID NO: 81 - protein, 28 aa, synthetic construct
GGGGSGGGGSLGGSGRSANAILEGGGGS

SEQ ID NO: 82 - protein, 21 aa, synthetic construct
GGGGSLGGSGRSANAILEGGS

SEQ ID NO: 83 - protein, 14 aa, synthetic construct
GGGSGPTNKVRGGS

SEQ ID NO: 84 - protein, 15 aa, synthetic construct
GGSGPLGMLSQGGGS

SEQ ID NO: 85 - protein, 10 aa, synthetic construct
GGPLGMLSQS

SEQ ID NO: 86 - protein, 13 aa, unidentified
GGGPLGMLSQGGS

SEQ ID NO: 87 - protein, 10 aa, synthetic construct
GGPTNKVRGS

SEQ ID NO: 88 - protein, 10 aa, synthetic construct
GRQARAVGGS

SEQ ID NO: 89 - protein, 15 aa, synthetic construct
GGGSGRSENIRTAGG

SEQ ID NO: 90 - protein, 15 aa, synthetic construct
SGGPGPAGMKGLPGS

SEQ ID NO: 91 - protein, 15 aa, synthetic construct
GGGGSPMAKKGGGGS

SEQ ID NO: 92 - protein, 15 aa, synthetic construct
GGPLGMLSQPMAKKS

SEQ ID NO: 93 - protein, 20 aa, synthetic construct
GGSGPLGMLSQPMAKKGGGS

SEQ ID NO: 94 - protein, 20 aa, synthetic construct
GGGPMAKKGPLGMLSQGGGS

SEQ ID NO: 95 - protein, 14 aa, synthetic construct
EPKSSDKTHTSPPS

SEQ ID NO: 96 - protein, 12 aa, synthetic construct
GGGSGGGSGGGS

SEQ ID NO: 97 - protein, 4 aa, synthetic construct
GGGS

SEQ ID NO: 98 - protein, 9 aa, synthetic construct
GSSGGSGGS

SEQ ID NO: 99 - protein, 5 aa, synthetic construct
GSSGT

SEQ ID NO: 100 - protein, 15 aa, synthetic construct
GGGGSGGGGSGGGGS

SEQ ID NO: 101 - protein, 17 aa, synthetic construct
AEAAAKEAAAKEAAAKA

SEQ ID NO: 102 - protein, 20 aa, synthetic construct
GGGGSGGGGSGGGGSGGGGS

SEQ ID NO: 103 - protein, 8 aa, synthetic construct
GGGSGGGS

SEQ ID NO: 104 - skipped

SEQ ID NO: 105 - protein, 4 aa, synthetic construct
GGSS

SEQ ID NO: 106 - protein, 5 aa, synthetic construct
GGGGS

SEQ ID NO: 107 - protein, 5 aa, synthetic construct
GGSGG

SEQ ID NO: 108 - protein, 4 aa, synthetic construct
SGGG

SEQ ID NO: 109 - protein, 4 aa, synthetic construct
GSGS

SEQ ID NO: 110 - protein, 6 aa, synthetic construct
GSGSGS

SEQ ID NO: 111 - protein, 8 aa, synthetic construct
GSGSGSGS

SEQ ID NO: 112 - protein, 10 aa, synthetic construct
GSGSGSGSGS

SEQ ID NO: 113 - protein, 12 aa, synthetic construct
GSGSGSGSGSGS

SEQ ID NO: 114 - protein, 10 aa, synthetic construct
GGGGSGGGGS

SEQ ID NO: 115 - protein, 15 aa, synthetic construct
GGGGSGGGGSGGGGS

SEQ ID NO: 116 - protein, 133 aa, Homo sapiens [sequence omitted]

SEQ ID NO: 117 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 118 - protein, 133 aa, synthetic construct
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 119 - protein, 133 aa, synthetic construct
APTSSSTKKTQLQLEHLLLDLQMILNGINNYKNPKLTRMLTFKFYMPKKATELKHLQCLEEELKKLEEVLNLAQSKNFHLRPRDLISNINVIVLELKGSETTFMCEYADETATIVEFLNRWITFIQSIISTLT

SEQ ID NO: 120 - protein, 133 aa, synthetic construct
APTSSSTKKTQLQLEHLLLDLQMILNGINNYKNPKLTRMLTFKFYMPKKATELKHLQCLEEELKNLEEVLNLAQSKNFHLRPRDLISNINVIVLELKGSETTFMCEYADETATIVEFLNRWITFIQSIISTLT

SEQ ID NO: 121 - protein, 133 aa, synthetic construct
APTSSSTKKTQLQLEHLLLDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIQSIISTLT

SEQ ID NO: 122 - protein, 133 aa, synthetic construct
APTSSSTKKTQLQLEHLLLDLQMILNGINNYKNPKLTRMLTFKFYMPKKATELKHLQCLEEELKHLEEVLNLAQSKNFHLRPRDLISNINVIVLELKGSETTFMCEYADETATIVEFLNRWITFIQSIISTLT

SEQ ID NO: 123 - protein, 133 aa, synthetic construct
APTSSSTKKTQLQLEHLLLDLQMILNGINNYKNPKLTRMLTFKFYMPKKATELKHLQCLEEELKGLEEVLNLAQSKNFHLRPRDLISNINVIVLELKGSETTFMCEYADETATIVEFLNRWITFIQSIISTLT

SEQ ID NO: 124 - protein, 133 aa, synthetic construct
APTSSSTKKTQLQLEHLLLDLQMILNGINNYKNPKLTRMLTFKFYMPKKATELKHLQCLEEELKELEEVLNLAQSKNFHLRPRDLISNINVIVLELKGSETTFMCEYADETATIVEFLNRWITFIQSIISTLT

SEQ ID NO: 125 - protein, 133 aa, synthetic construct
APTSSSTKKTQLQLEHLLLDLQMILNGINNYKNPKLTRMLTFKFYMPKKATELKHLQCLEEELKALEEVLNLAQSKNFHLRPRDLISNINVIVLELKGSETTFMCEYADETATIVEFLNRWITFIQSIISTLT

SEQ ID NO: 126 - protein, 133 aa, synthetic construct
APTSSSTKKTQLQLEHLLHDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 127 - protein, 133 aa, synthetic construct
APTSSSTKKTQLQLEHLLQDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 128 - protein, 133 aa, synthetic construct
APTSSSTKKTQLQLEHLLYDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 129 - protein, 133 aa, synthetic construct
APTSSSTKKTQLQLEHLLDDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 130 - protein, 133 aa, synthetic construct
APTSSSTKKTQLQLEHLLSDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 131 - protein, 133 aa, synthetic construct
APTSSSTKKTQLQLEHLLNDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 132 - protein, 133 aa, synthetic construct
APTSSSTKKTQLQLEHLLRDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIQSIISTLT

SEQ ID NO: 133 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIASIISTLT

SEQ ID NO: 134 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIDSIISTLT

SEQ ID NO: 135 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIESIISTLT

SEQ ID NO: 136 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIFSIISTLT

SEQ ID NO: 137 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIGSIISTLT

SEQ ID NO: 138 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIHSIISTLT

SEQ ID NO: 139 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIISIISTLT

SEQ ID NO: 140 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIKSIISTLT

SEQ ID NO: 141 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFILSIISTLT

SEQ ID NO: 142 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIMSIISTLT

SEQ ID NO: 143 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFINSIISTLT

SEQ ID NO: 144 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIPSIISTLT

SEQ ID NO: 145 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIRSIISTLT

SEQ ID NO: 146 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFISSIISTLT

SEQ ID NO: 147 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFITSIISTLT

SEQ ID NO: 148 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIVSIISTLT

SEQ ID NO: 149 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIWSIISTLT

SEQ ID NO: 150 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFIYSIISTLT

SEQ ID NO: 151 - protein, 133 aa, synthetic construct
APTSSSTKKTQLQLEHLLHDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 152 - protein, 133 aa, synthetic construct
APTSSSTKKTQLQLEHLLQDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 153 - protein, 133 aa, synthetic construct
APTSSSTKKTQLQLEHLLYDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 154 - protein, 133 aa, synthetic construct
APTSSSTKKTQLQLEHLLQDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIQSIISTLT

SEQ ID NO: 155 - protein, 133 aa, synthetic construct
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIASIISTLT

SEQ ID NO: 156 - protein, 133 aa, synthetic construct
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIDSIISTLT

SEQ ID NO: 157 - protein, 133 aa, synthetic construct
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIESIISTLT

SEQ ID NO: 158 - protein, 133 aa, synthetic construct
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIFSIISTLT

SEQ ID NO: 159 - protein, 133 aa, synthetic construct
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIGSIISTLT

SEQ ID NO: 160 - protein, 133 aa, synthetic construct
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIHSIISTLT

SEQ ID NO: 161 - protein, 133 aa, synthetic construct
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIISIISTLT

SEQ ID NO: 162 - protein, 133 aa, synthetic construct
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIKSIISTLT

SEQ ID NO: 163 - protein, 133 aa, synthetic construct
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFILSIISTLT

SEQ ID NO: 164 - protein, 133 aa, synthetic construct
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIMSIISTLT

SEQ ID NO: 165 - protein, 133 aa, synthetic construct
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFINSIISTLT

SEQ ID NO: 166 - protein, 133 aa, synthetic construct
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIPSIISTLT

SEQ ID NO: 167 - protein, 133 aa, synthetic construct
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIRSIISTLT

SEQ ID NO: 168 - protein, 133 aa, synthetic construct
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFISSIISTLT

SEQ ID NO: 169 - protein, 133 aa, synthetic construct
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFITSIISTLT

SEQ ID NO: 170 - protein, 133 aa, synthetic construct
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIVSIISTLT

SEQ ID NO: 171 - protein, 133 aa, synthetic construct
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIWSIISTLT

SEQ ID NO: 172 - protein, 133 aa, synthetic construct
APTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIYSIISTLT

SEQ ID NO: 173 - protein, 133 aa, synthetic construct
APTSSSTKKTQLQLEHLLYDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFINSIISTLT

SEQ ID NO: 174 - protein, 133 aa, synthetic construct; identical to SEQ ID NO: 173

SEQ ID NO: 175 - protein, 133 aa, synthetic construct
APTSSSTKKTQLQLEHLLYDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFISSIISTLT

SEQ ID NO: 176 - protein, 133 aa, synthetic construct
APTSSSTKKTQLQLEHLLLDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFINSIISTLT

SEQ ID NO: 177 - protein, 133 aa, synthetic construct
APTSSSTKKTQLQLEHLLLDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIHSIISTLT

SEQ ID NO: 178 - protein, 133 aa, synthetic construct
APTSSSTKKTQLQLEHLLLDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIMSIISTLT

SEQ ID NO: 179 - protein, 133 aa, synthetic construct
APTSSSTKKTQLQLEHLLLDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIFSIISTLT

SEQ ID NO: 180 - protein, 133 aa, synthetic construct
APTSSSTKKTQLQLEHLLLDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIRSIISTLT

SEQ ID NO: 181 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 182 - protein, 165 aa, Homo sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 183 - protein, 165 aa, synthetic construct
ELCDDDPPEIPHATFKAMAYKEGTMLNCECKRGFRRIKSGSLAMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 184 - protein, 165 aa, synthetic construct
ELCDDDPPEIPHATFKAMAYKEGTMLNCECKRGFRRIKSGSGYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 185 - protein, 165 aa, synthetic construct
ELCDDDPPEIPHATFKAMAYKEGTMLNCECKRGFRAIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 186 - protein, 226 aa, synthetic construct
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 187 - protein, 226 aa, synthetic construct
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 188 - protein, 226 aa, synthetic construct
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 189 - protein, 443 aa, Mus musculus
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPG

SEQ ID NO: 190 - protein, 443 aa, Mus musculus
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPG

SEQ ID NO: 191 - protein, 443 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWDGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 192 - protein, 443 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWDGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 193 - protein, 215 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 194 - protein, 767 aa, synthetic construct
EVQLVESGGGLVKPGGSLRLSCAASGFTFSSYDMSWVRQAPGKGLEWVATISGGGSYTYYPDSVKGRFTISRDNAKNSLYLQMNSLRAEDTAVYYCASPDSSGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIQSIISTLTGGSGPLGMLSQGGGSELCDDDPPEIPHATFKAMAYKEGTMLNCECKRGFRRIKSGSGYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 195 - protein, 446 aa, synthetic construct
EVQLVESGGGLVKPGGSLRLSCAASGFTFSSYDMSWVRQAPGKGLEWVATISGGGSYTYYPDSVKGRFTISRDNAKNSLYLQMNSLRAEDTAVYYCASPDSSGVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 196 - protein, 770 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIQSIISTLTGGSGPLGMLSQGGGSELCDDDPPEIPHATFKAMAYKEGTMLNCECKRGFRRIKSGSGYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 197 - protein, 450 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGG

SEQ ID NO: 198 - protein, 770 aa, synthetic construct
QVQLVQSGVEVKKPGASVKVSCKASGYTFTNYYMYWVRQAPGQGLEWMGGINPSNGGTNFAQKFQGRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIQSIISTLTGGSGPLGMLSQGGGSELCDDDPPEIPHATFKAMAYKEGTMLNCECKRGFRRIKSGSGYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 199 - protein, 449 aa, synthetic construct
QVQLVQSGVEVKKPGASVKVSCKASGYTFTNYYMYWVRQAPGQGLEWMGGINPSNGGTNFAQKFQGRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 200 - protein, 770 aa, synthetic construct
EVQLLESGGGLVQPGGSLRLSCAASGFTFTNYYMYWVRQAPGKGLEWMGGINPSNGGTNYADKFKGRFTLSTDSSKNTLYLQMNSLRAEDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIQSIISTLTGGSGPLGMLSQGGGSELCDDDPPEIPHATFKAMAYKEGTMLNCECKRGFRRIKSGSGYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 201 - protein, 449 aa, synthetic construct
EVQLLESGGGLVQPGGSLRLSCAASGFTFTNYYMYWVRQAPGKGLEWMGGINPSNGGTNYADKFKGRFTLSTDSSKNTLYLQMNSLRAEDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 202 - protein, 770 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIQSIISTLTGGSGPLGMLSQGGGSELCDDDPPEIPHATFKAMAYKEGTMLNCECKRGFRRIKSGSGYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 203 - protein, 449 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 204 - protein, 770 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFINSIISTLTGGSGPLGMLSQGGGSELCDDDPPEIPHATFKAMAYKEGTMLNCECKRGFRRIKSGSGYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 205 - protein, 770 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIHSIISTLTGGSGPLGMLSQGGGSELCDDDPPEIPHATFKAMAYKEGTMLNCECKRGFRRIKSGSGYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 206 - protein, 770 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIMSIISTLTGGSGPLGMLSQGGGSELCDDDPPEIPHATFKAMAYKEGTMLNCECKRGFRRIKSGSGYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 207 - protein, 770 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIFSIISTLTGGSGPLGMLSQGGGSELCDDDPPEIPHATFKAMAYKEGTMLNCECKRGFRRIKSGSGYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 208 - protein, 770 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLLDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIRSIISTLTGGSGPLGMLSQGGGSELCDDDPPEIPHATFKAMAYKEGTMLNCECKRGFRRIKSGSGYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 209 - protein, 770 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSAPTSSSTKKTQLQLEHLLQDLQMILNGINNYKNPKLTRMLTFKFYMPKKATELKHLQCLEEELKQLEEVLNLAQSKNFHLRPRDLISNINVIVLELKGSETTFMCEYADETATIVEFLNRWITFIQSIISTLTGGSGPLGMLSQGGGSELCDDDPPEIPHATFKAMAYKEGTMLNCECKRGFRRIKSGSGYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 210 - protein, 592 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSAPTSSSTKKTQLQLEHLLQDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 211 - protein, 592 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSAPTSSSTKKTQLQLEHLLHDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT

SEQ ID NO: 212 - protein, 592 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSAPTSSSTKKTQLQLEHLLQDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIHSIISTLT

SEQ ID NO: 213 - protein, 592 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSAPTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIGSIISTLT

SEQ ID NO: 214 - protein, 592 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSAPTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFISSIISTLT

SEQ ID NO: 215 - protein, 592 aa, synthetic construct
QVQLVQSGAEVKKPGASVKVSCKASGYTFTNYYMYWVRQAPGQGLEWMGGINPSNGGTNFAQKFQGRVTLTTDSSTSTAYMELSSLRSDDTAVYYCARRDYRFDMGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSAPTSSSTKKTQLQLEHLLLDLQMILNGINNYKNPKLTRMLTFKFYMPKKATELKHLQCLEEELKRLEEVLNLAQSKNFHLRPRDLISNINVIVLELKGSETTFMCEYADETATIVEFLNRWITFIQSIISTLT